(19) United States
(12) Patent Application Publication
(10) Pub. No.: US 2011/0046142 A1
(43) Pub. Date: Feb. 24, 2011

(54) ANTIBIOTIC COMPOUNDS

(75) Inventors: Kim Lewis, Newton, MA (US); Gabriele Casadei, Cambridge, MA (US)

Correspondence Address: WILMERHALE/BOSTON, 60 STATE STREET, BOSTON, MA, 02109, US

(73) Assignee: Northeastern University, Boston, MA (US)

(21) Appl. No.: 12/664,250

(22) PCT Filed: Jun. 11, 2008
(86) PCT No.: PCT/US08/07290

§ 371 Date: Nov. 1, 2010

Related U.S. Application Data
(60) Provisional application No. 60/934,418, filed on Jun. 13, 2007.

(51) Int. Cl. A61K 31/53; A61K 31/34; A61K 31/381; A61K 31/496; A61K 31/436; A61K 31/498; A61K 31/47; A61K 31/407; A61P 31/00; A61P 31/04; A61P 31/10; A61P 33/02; A61P 17/02; A61P 11/00; A61P 9/00

(52) U.S. Cl. 514/246; 514/472; 514/438; 514/254.11; 514/254.04; 514/291; 514/249; 514/313; 514/411

(57) ABSTRACT

Methods for identifying prodrug antibiotic compounds and direct inhibitory antibiotic compounds utilizing various screens are provided. Also provided are methods for treating infections using these compounds.

### CROSS-REFERENCE TO RELATED APPLICATIONS

This application claims the benefit of U.S. Provisional Application No. 60/934,418 filed Jun. 13, 2007, the entire contents of which are hereby incorporated by reference herein.

### FIELD OF THE INVENTION

The invention is in the fields of microbial chemistry and medicinal chemistry.

### BACKGROUND OF THE INVENTION

A renewed focus on antimicrobial drug discovery is important as pathogens become increasingly more resistant to available drugs (Lewis et al. (2002) Drug Efflux. In Bacterial Resistance to Antimicrobials: Mechanisms, Genetics, Medical Practice and Public Health. Lewis, et al. (eds). New York: Marcel Dekker, pp. 61-90; Levy et al. (2004) Nat. Med. 10:S122-129). Further, there exists a need in antimicrobial drug discovery for novel broad-spectrum compounds, because in many cases, there is not enough time to identify the exact nature of a pathogen. This is especially true, for example, in the case of a bioterrorist attack.

Synthetic compounds have thus far failed to replace natural antibiotics despite the combined efforts of combinatorial synthesis, high-throughput screening, advanced medicinal chemistry, genomics and proteomics, and rational drug design. As a result, many companies have closed their anti-infective divisions (see, e.g., Silver (2005) IDrugs 8(8):651-655).

The problem with obtaining new synthetic antibiotics may be related in part to the synthetic antibiotics being pumped out across the outer membrane barrier of Gram-negative bacteria by Multidrug Resistance pumps (MDRs). The outer membrane of Gram-negative bacteria is a barrier for amphipathic compounds, and MDRs extrude drugs across this barrier. Although natural antibiotics can largely bypass this dual barrier/extrusion mechanism, many synthetic compounds cannot.

Apart from broad-spectrum compounds, there is an even greater unmet need for sterilizing antimicrobials. The inability to sterilize an infection is recognized as a shortcoming of antibiotics, and there appear to be no approaches to develop such compounds (Lewis (2001a)) Chemother. 45:999-1007; Coates et al. (2002) Nat. Rev. Drug Discov. 1:895-910). The FDA only requires testing of rapidly propagating cultures for drug approval. Yet, the unmet need for compounds that can eradicate, rather than suppress, an infection and be effective against slow-growing biofilm infections is acute.

### SUMMARY OF THE INVENTION

The invention is based, at least in part, on the discovery of compounds having prodrug or direct antibiotic activity. Accordingly, in one aspect, the invention features compounds of the Formula I:

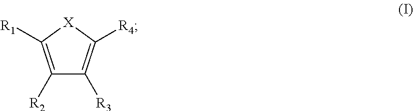

and pharmaceutically acceptable salts, hydrates, and solvates thereof,
wherein:

R₁ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein one or more hydrogens on R₁ can be substituted with 0-5 Rₐ groups;

R₂ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein one or more hydrogens on R₂ can be substituted with 0-5 Rₐ groups;

R₃ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, —CH═CHNO₂, —CH₂SC(NH)NH₂, wherein one or more hydrogens on R₃ can be substituted with 0-5 Rₐ groups;

R₄ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl,

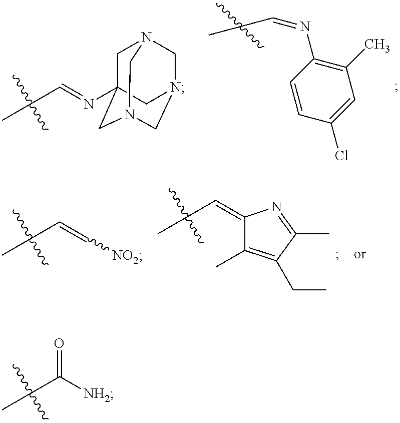

wherein one or more hydrogens on R₄ can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

X is O, S, or NH; and

wherein the compound is not 5-bromo-N-phenylthiophene-2-carboxamide, 1,3,5-triazatricyclo[3.3.3.1.1]decan-7-amine, N-[(5-nitro-2-thienyl)methylene], 4-chloro-2-methyl-N-((5-nitrofuran-2-yl)methylene)aniline, 4-bromo-2-(2-nitrovinyl)thiophene, 3-(2-nitrovinyl)thiophene, (E)-3-ethyl-5-((4-ethyl-3,5-dimethyl-2H-pyrrol-2-ylidene)methyl)-2,4-dimethyl-1H-pyrrole, (4,5,6,7-tetrahydrobenzo[b]thiophen-3-yl)methyl carbamimidothioate, or 5-nitrofuran-2-carboxamide.

In one embodiment, the compound is an analog of Compound 1

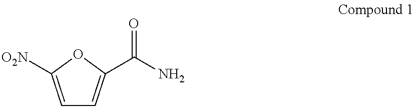

or a pharmaceutically acceptable salt, hydrate, or solvate thereof, wherein:

any one or more of —H and —NO₂, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl;

any one or more of —H attached to nitrogen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and the analog is not 5-nitrofuran-2-carboxamide.

In another embodiment, the compound is an analog of Compound 2

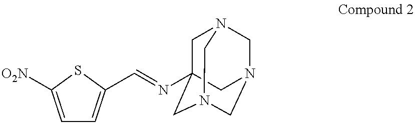

or a pharmaceutically acceptable salt, hydrate, or solvate thereof,

wherein any one or more of —H and —NO₂ attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration; and wherein the analog is not 1,3,5-triazatricyclo[3.3.3.1.1]decan-7-amine, N-[(5-nitro-2-thienyl)methylene].

In another embodiment, the compound is an analog of Compound 3

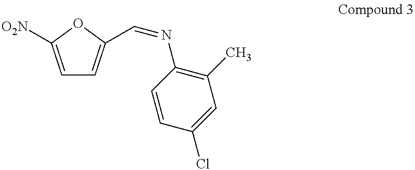

or a pharmaceutically acceptable salt, hydrate, or solvate thereof,

wherein any one or more of —H, —Cl, and —CH₃ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration; and wherein the analog is not (Z)-4-chloro-2-methyl-N-((5-nitrofuran-2-yl)methylene)aniline.

In yet another embodiment, the compound is an analog of Compound 4

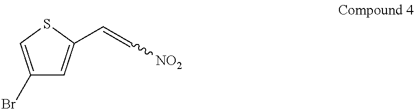

or a pharmaceutically acceptable salt, hydrate, or solvate thereof,

wherein any one or more of —H and —Br can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration; and wherein the analog is not 4-bromo-2-(2-nitrovinyl)thiophene.

In another embodiment, the compound is an analog of Compound 5

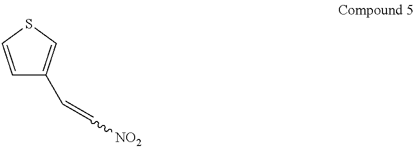

or a pharmaceutically acceptable salt, hydrate, or solvate thereof,

wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and wherein the analog is not 3-(2-nitrovinyl)thiophene.

In another aspect, the invention features compounds of Formula II:

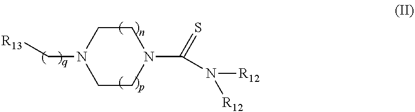

and pharmaceutically acceptable salts, hydrates, and solvates thereof,
wherein:

each R₁₂, independently, is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl,

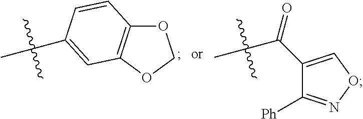

wherein one or more hydrogens on R₁₂ can be substituted with 0-5 Rₐ groups;

R₁₃ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl,

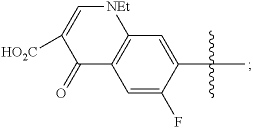

or
wherein one or more hydrogens on R₁₃ can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆cycloalkyl, or SOₓC₃₋₆ cycloalkyl-C₁₋₃ alkyl;

n is 0 or 1;

p is 1 or 2;

q is 0 or 1; and

the compound is not 3-(3-chlorobenzyl)-N-(3-chlorophenyl)tetrahydropyrimidine-1(2H)-carbothioamide, 7-(4-(benzo[d][1,3]dioxol-5-ylcarbamothioyl)piperazin-1-yl)-1-ethyl-6-fluoro-4-oxo-1,4-dihydroquinoline-3-carboxylic acid, or 1-ethyl-6-fluoro-4-oxo-7-(4-(3-phenylisoxazole-4-carbonylcarbamothioyl)piperazin-1-yl)-1,4-dihydroquinoline-3-carboxylic acid.

In one embodiment, the compound is an analog of Compound 6

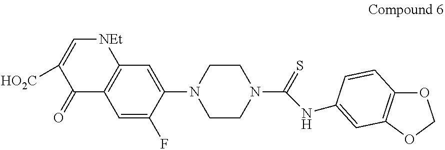

or a pharmaceutically acceptable salt, hydrate, or solvate thereof, wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₂CH₃, attached to nitrogen or oxygen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and

—H attached to oxygen can be substituted with any one of the following substituents: C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl; and wherein the analog is not 7-(4-(benzo[d][1,3]dioxol-5-ylcarbamothioyl)piperazin-1-yl)-1-ethyl-6-fluoro-4-oxo-1,4-dihydroquinoline-3-carboxylic acid.

In one embodiment, the compound is an analog of Compound 7

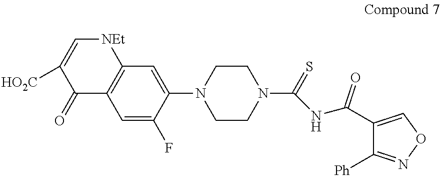

or a pharmaceutically acceptable salt, hydrate, or solvate thereof, wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₂CH₃, attached to nitrogen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

—H attached to oxygen can be substituted with any one of the following substituents: C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl; and

the phenyl attached to the isoxazole can be replaced with C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl; and wherein the analog is not 1-ethyl-6-fluoro-4-oxo-7-(4-(3-phenylisoxazole-4-carbonylcarbamothioyl)piperazin-1-yl)-1,4-dihydroquinoline-3-carboxylic acid.

In another aspect, the invention features analogs of Compound 8

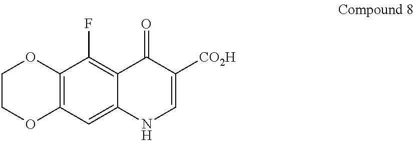

and pharmaceutically acceptable salts, hydrates, and solvates thereof, wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and

the analog is not 2,3,6,9-tetrahydro-9-oxo-1,4-dioxino[2,3-g]quinoline-8-carboxylic acid.

In another aspect, the invention features analogs of Compound 9

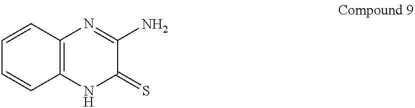

and pharmaceutically acceptable salts, hydrates, and solvates thereof, wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the C(S) can be substituted with C(O); and

the analog is not 3-aminoquinoxaline-2(1H)-thione.

In another aspect, the invention features analogs of Compound 10

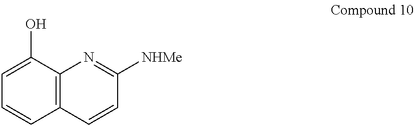

and pharmaceutically acceptable salts, hydrates, and solvates thereof, wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₃ attached to nitrogen or oxygen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and

the analog is not 2-(methylamino)quinolin-8-ol.

In another aspect, the invention features analogs of Compound 11

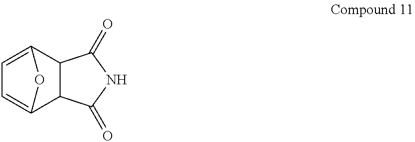

and pharmaceutically acceptable salts, hydrates, and solvates thereof, wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₃ attached to nitrogen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl, C₃₋₆ cycloalkyl; C₃₋₆ cycloalkyl-C₁₋₃ alkyl; alkylaryl; aryl; arylalkyl; and heteroaryl; and

the analog is not 4,7-epoxy-1H-isoindole-1,3(2H)-dione.

In another aspect, the invention features a method of inhibiting the growth of, or killing, a pathogen, the method comprising contacting the pathogen with one or more compounds of Formulae I and II, or a pharmaceutically acceptable salt, hydrate, or solvate thereof

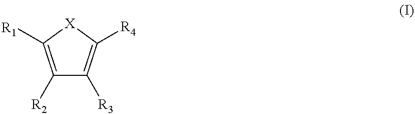

wherein:

R₁ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein one or more hydrogens on R₁ can be substituted with 0-5 Rₐ groups;

R₂ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein one or more hydrogens on R₂ can be substituted with 0-5 Rₐ groups;

R₃ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, —CH═CHNO₂, —CH₂SC(NH)NH₂, wherein one or more hydrogens on R₃ can be substituted with 0-5 Rₐ groups;

R₄ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl,

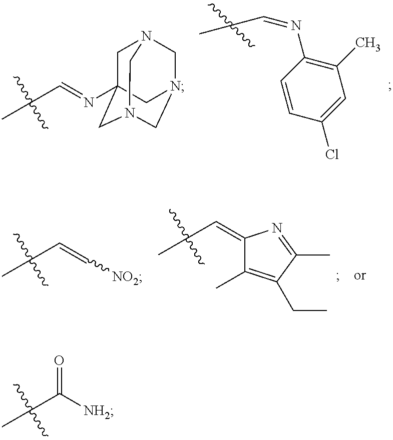

wherein one or more hydrogens on R₄ can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋6 cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl; and

X is O, S, or NH;

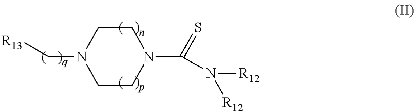

wherein:

each R₁₂, independently, is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl,

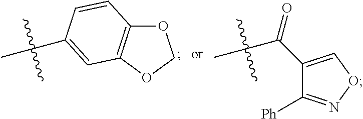

wherein one or more hydrogens on R₁₂ can be substituted with 0-5 Rₐ groups;

R₁₃ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl,

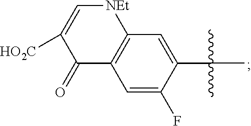

or
wherein one or more hydrogens on R₁₃ can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋6 cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

n is 0 or 1;

p is 1 or 2;

q is 0 or 1; and

wherein contacting the pathogen with one or more compounds of Formulae I and II inhibits the growth of, or kills, a pathogen.

In certain embodiments, the compound is a compound having Formula I. In other embodiments, the compound is a compound having Formula II.

In some embodiments, the pathogen is one or more of a bacterium, a fungus, a protozoan, or a helminth. In certain embodiments, the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

In another aspect, the invention features a method of treating an infection by a pathogen in a subject in need thereof, the method comprising administering to the subject an effective amount of one or more compounds of Formulae I and II, or a pharmaceutically acceptable salt, hydrate, or solvate thereof,

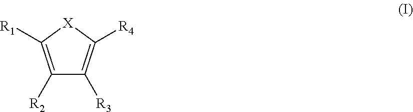

wherein:

R₁ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃alkyl, aryl, arylalkyl, heteroaryl, or heteroarylalkyl, wherein one or more hydrogens on R₁ can be substituted with 0-5 Rₐ groups;

R₂ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein one or more hydrogens on R₂ can be substituted with 0-5 Rₐ groups;

R₃ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, —CH═CHNO₂, —CH₂SC(NH)NH₂, wherein one or more hydrogens on R₃ can be substituted with 0-5 Rₐ groups;

R₄ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl,

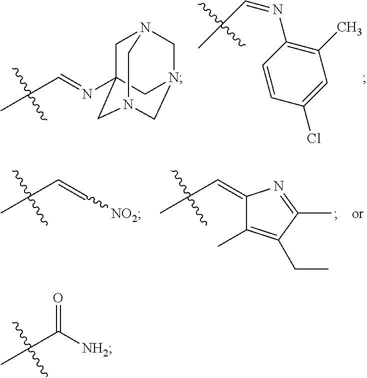

wherein one or more hydrogens on R₄ can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl; and

X is O, S, or NH;

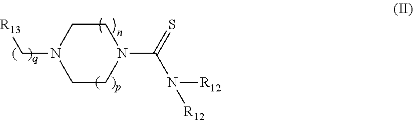

wherein:

each R₁₂, independently, is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl,

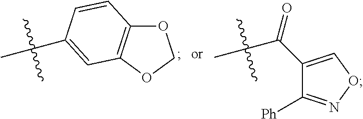

wherein one or more hydrogens on R₁₂ can be substituted with 0-5 Rₐ groups;

R₁₃ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl,

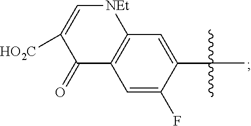

or
wherein one or more hydrogens on R₁₃ can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

n is 0 or 1;

p is 1 or 2;

q is 0 or 1;

thereby treating the infection.

In certain embodiments, the compound is a compound having Formula I. In other embodiments, the compound is a compound having Formula II.

In some embodiments, the pathogen is one or more of a bacterium, a fungus, a protozoan, and a helminth. In certain embodiments, the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms. In some embodiments, the infection by the pathogen is an upper respiratory tract disease, an infection of a catheter, an infection of an orthopedic prostheses, a urinary tract infection, a gastrointestinal infection, a heart valve infection, endocarditis, a skin infection, a chronic wound, or cystic fibrosis.

In another aspect, the invention features a method of inhibiting the growth of, or eradicating, a pathogenic agent by contacting the pathogen with one or more analogs of Compounds 1-11, or a pharmaceutically acceptable salt, hydrate, or solvate thereof:

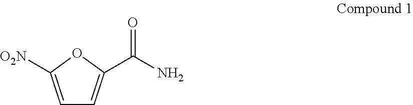

wherein any one or more of —H and —NO₂, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl;

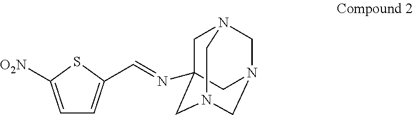

wherein any one or more of —H and —NO₂ attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl, —C(O)OC₁₋₆alkyl; C₃₋₆ cycloalkyl; C₃₋₆ cycloalkyl-C₁₋₃ alkyl; alkylaryl; aryl; arylalkyl; heteroaryl; the double bond can be in the E or Z configuration;

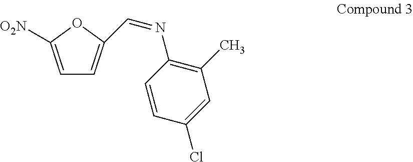

wherein any one or more of —H, —Cl, and —CH₃ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration;

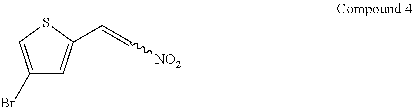

wherein any one or more of —H and —Br can be substituted with any one of the following substituents: —H, halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration;

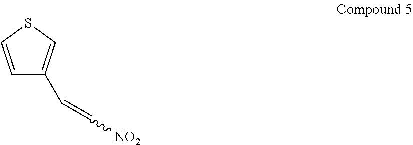

wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

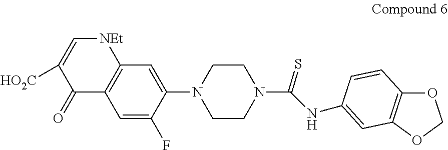

wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₂CH₃, attached to nitrogen or oxygen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

—H attached to oxygen can be substituted with any one of the following substituents: C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl;

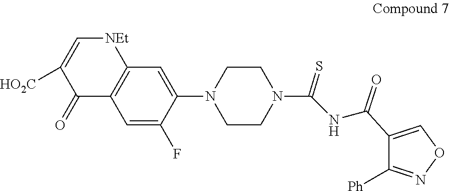

wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₂CH₃, attached to nitrogen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

—H attached to oxygen can be substituted with any one of the following substituents: C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl;

the phenyl attached to the isoxazole can be replaced with C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl;

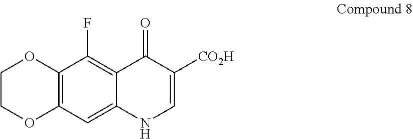

wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

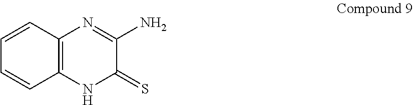

wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the C(S) can be substituted with C(O);

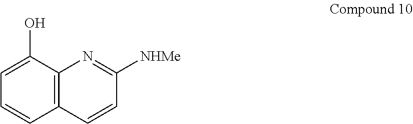

wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₃ attached to nitrogen or oxygen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

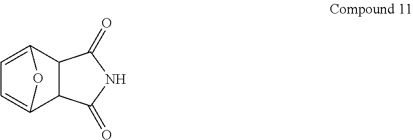

wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₃ attached to nitrogen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and wherein the analog is not 4,7-epoxy-1H-isoindole-1,3(2H)-dione; and

any one or more of —H attached to nitrogen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

thereby inhibiting the growth of, or eradicating, the pathogen.

In certain embodiments, the pathogen is one or more of a bacterium, a fungus, a protozoan, or a helminth. In some embodiments, the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

In another aspect, the invention features a method of treating an infection by a pathogen in a subject in need thereof, the method comprising administering to the subject an effective amount of one or more analogs of Compounds 1-11, or a pharmaceutically acceptable salt, hydrate, or solvate thereof,

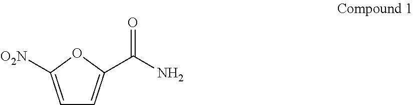

wherein any one or more of —H and —NO₂, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl;

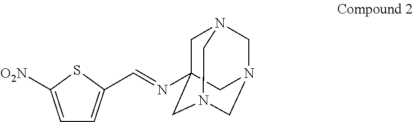

wherein any one or more of —H and —NO₂ attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration;

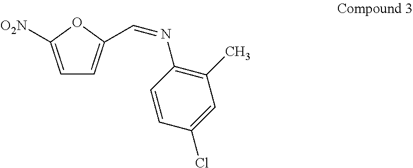

wherein any one or more of —H, —Cl, and —CH₃ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration;

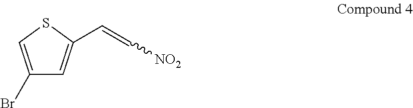

wherein any one or more of —H and —Br can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl, —C(O)OC₁₋₆alkyl; C₃₋₆ cycloalkyl, C₃₋₆ cycloalkyl-C₁₋₃ alkyl; alkylaryl; aryl; arylalkyl; and heteroaryl; the double bond can be in the E or Z configuration;

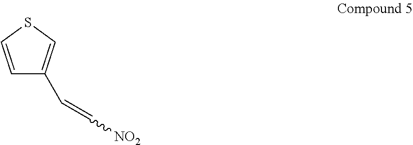

wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

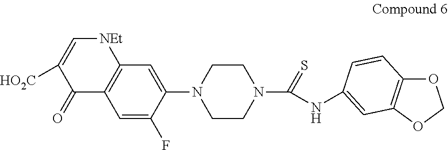

wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₂CH₃, attached to nitrogen or oxygen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

—H attached to oxygen can be substituted with any one of the following substituents: C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl;

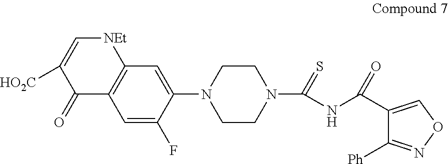

wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₂CH₃, attached to nitrogen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

—H attached to oxygen can be substituted with any one of the following substituents: C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl;

the phenyl attached to the isoxazole can be replaced with C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl;

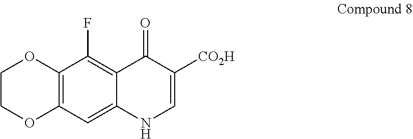

wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

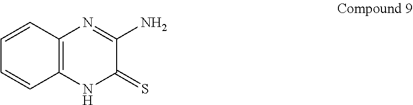

wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the C(S) can be substituted with C(O);

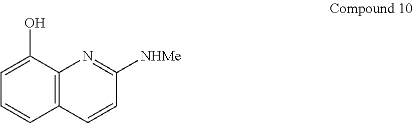

wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₃ attached to nitrogen or oxygen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl, C₃₋₆ cycloalkyl; C₃₋₆ cycloalkyl-C₁₋₃ alkyl; alkylaryl; aryl; arylalkyl; and heteroaryl;

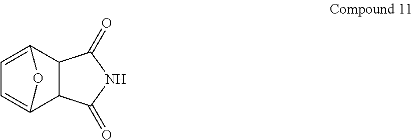

wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₃ attached to nitrogen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and

any one or more of —H attached to nitrogen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; aryl alkyl; and heteroaryl;

thereby treating the pathogenic infection.

In certain embodiments, the pathogen is one or more of a bacterium, a fungus, a protozoan, or a helminth. In some embodiments, the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

In some embodiments, the infection by the pathogen is an upper respiratory tract disease, an infection of a catheter, an infection of an orthopedic prostheses, a urinary tract infection, a gastrointestinal infection, a heart valve infection, endocarditis, a skin infection, a chronic wound, or cystic fibrosis.

In another aspect, the invention features a method of inhibiting the growth of, or killing, a pathogen, the method comprising contacting the pathogen with an effective amount of one or more of Compounds 1-11:

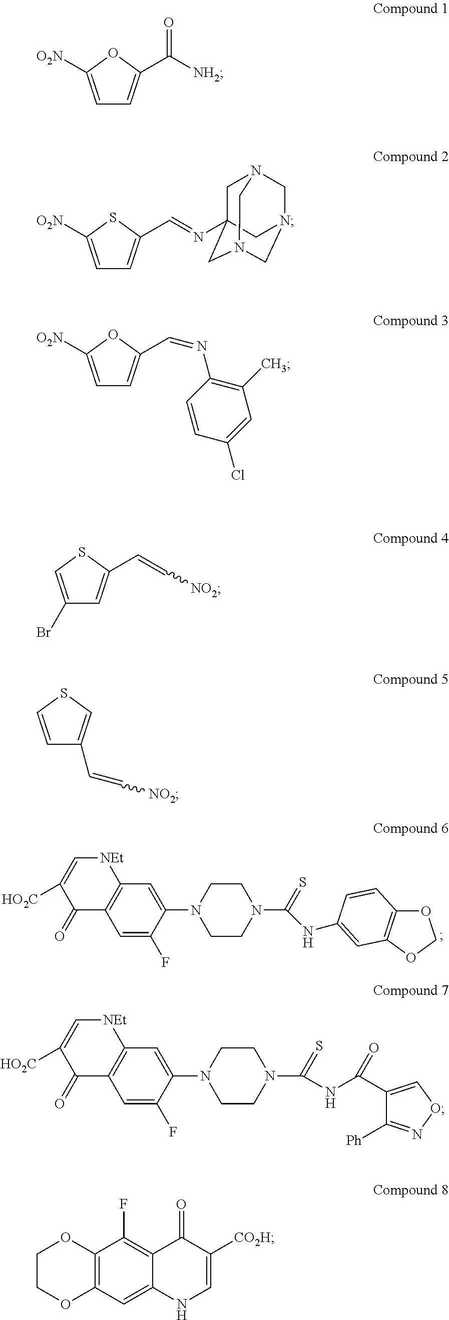

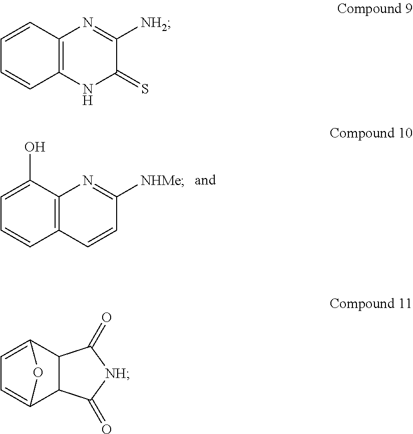

or a pharmaceutically acceptable salt, hydrate, or solvate of Compounds 1-11;

thereby inhibiting the growth of, or killing, the pathogen.

In certain embodiments, the pathogen is one or more of a bacterium, a fungus, a protozoan, or a helminth. In some embodiments, the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

In another aspect, the invention features a method of treating an infection by a pathogen in a subject in need thereof, the method comprising administering to the subject an effective amount of one or more of Compounds 1-11:

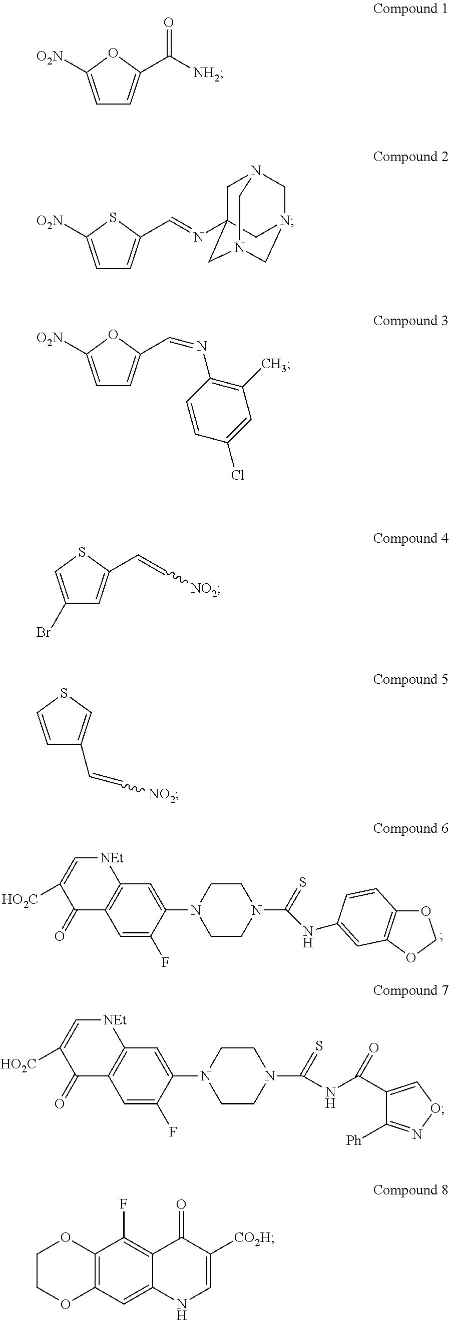

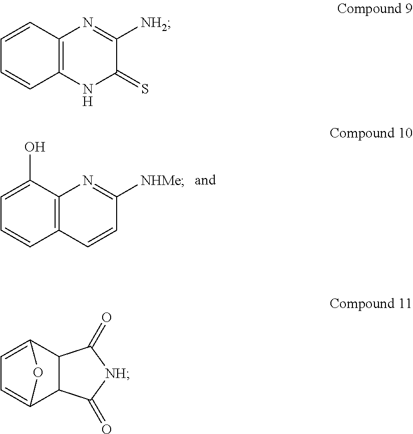

a pharmaceutically acceptable salt, hydrate, or solvate of Compounds 1-11;

thereby treating the infection.

In other aspects, the invention features pharmaceutical compositions comprising: compounds of Formula (I) or Formula (II); or pharmaceutically acceptable salts, hydrates, or solvates of compounds of Formula (I) or Formula (II); and a pharmaceutically acceptable carrier.

In other aspects, the invention features methods of sterilizing or killing persister cells, comprising contacting the persister cells with an effective amount of a compound of Formula (I) or Formula (II), or a pharmaceutically acceptable salt of a compound of Formula (I) or Formula (II).

In yet other aspects, the invention features pharmaceutical compositions comprising: one or more analogs of Compounds 1-11 or pharmaceutically acceptable salts, hydrates, or solvates of analogs of Compounds 1-11; and a pharmaceutically acceptable carrier.

In other aspects, the invention features methods of sterilizing or killing persister cells, comprising contacting the persister cells with an effective amount of one or more of an analog of Compounds 1-11 or a pharmaceutically acceptable salt of an analog of Compounds 1-11.

In other aspects, the invention features pharmaceutical compositions comprising: one or more of Compounds 1-11, or pharmaceutically acceptable salts, hydrates, or solvates of Compounds 1-11; and a pharmaceutically acceptable carrier.

In other aspects, the invention features methods of sterilizing or killing persister cells, comprising contacting the persister cells with an effective amount of one or more of Compounds 1-11, or a pharmaceutically acceptable salt of one or more of Compounds 1-11.

### DEFINITIONS

As used herein, a “prodrug” is a compound that is converted inside a cell of a pathogen into a reactive molecule which binds to one or more targets and modulates or impairs the activity of the cell.

As used herein, an “antibiotic” is a natural or synthetic compound that inhibits the growth of, or kills, a microorganism (e.g., bacterium, protozoan, fungus). In some instances, the antibiotic is active in inhibiting the growth of or killing other organisms such as helminths.

As used herein, a “broad-spectrum antibiotic” is an antibiotic that inhibits and/or kills a member of two or more different genuses of a microorganism. For example, an antibiotic that inhibits the growth of and/or kills both E. coli and M. tuberculosis is considered a broad-spectrum antibiotic. Similarly, an antibiotic that kills both S. cerevisiae and C. albicans is considered a broad-spectrum antibiotic.

As used herein, a “sterilizing antibiotic” is an antibiotic that kills both the growing cells in a population as well as persister cells.

As used herein, “persister cells” are antibiotic-tolerant cells produced stochastically by microbial populations.

As used herein, “sterilize a microbial population” means to kill a microbial population in the organism the microbe has infected, thereby substantially decreasing or preventing a relapse of the infection by the microbe. For example sterilizing an E. coli O157 population means to kill this pathogenic bacterium in the organism it has infected, thereby reducing or preventing relapse of infection by this pathogen.

As used herein, an “essential gene” is a gene that is essential to the survival of an organism in a specific environment. Thus, a gene may be essential for survival of a pathogenic organism within the organism it infects (i.e., essential in vivo) but not outside the organism it infects (in vitro).

As used herein, a population of microbes with “singular mutational identities” means a population of microbes that have mutations in a single genes. For example, a population of bacteria with singular mutational identities means a population of bacteria wherein each bacterium has a mutation in a single gene.

“Alkyl” refers to a hydrocarbon chain that may be a straight chain or branched chain, containing the indicated number of carbon atoms. For example, C₁-C₆ indicates that the group may have from 1 to 6 (inclusive) carbon atoms in it.

“Aryl” refers to cyclic aromatic carbon ring systems made from 6 to 18 carbons. Examples of an aryl group include, but are not limited to, phenyl, napthyl, anthracenyl, tetracenyl, and phenanthrenyl. An aryl group can be unsubstituted or substituted with one or more of the following groups: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Heteroaryl” refers to mono and bicyclic aromatic groups of 4 to 10 atoms containing at least one heteroatom. Heteroatom as used in the term heteroaryl refers to oxygen, sulfur and nitrogen. Examples of monocyclic heteroaryls include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, and pyrimidinyl. Examples of bicyclic heteroaryls include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Arylalkyl” refers to an aryl group with at least one alkyl substitution. Examples of arylalkyl include, but are not limited to, toluenyl, phenylethyl, xylenyl, phenylbutyl, phenylpentyl, and ethylnapthyl. An arylalkyl group can be unsubstituted or substituted with one or more of the following groups: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Heteroarylalkyl” refers to a heteroaryl group with at least one alkyl substitution. A heteroarylalkyl group can be unsubstituted or substituted with one or more of the following: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, isopropyl, n-pentyl, isopentyl, neopentyl, and hexyl.

“C₂-C₆ alkenyl” refers to a straight or branched chain unsaturated hydrocarbon containing 2-6 carbon atoms and at least one double bond. Examples of a C₂-C₆ alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, and isohexene.

“C₃-C₆ alkenyl” refers to a straight or branched chain unsaturated hydrocarbon containing 3-6 carbon atoms and at least one double bond. Examples of a C₃-C₆ alkenyl group include, but are not limited to, propylene, 1-butylene, 2-butylene, isobutylene, sec-butylene, 1-pentene, 2-pentene, isopentene, 1-hexene, 2-hexene, 3-hexene, and isohexene.

“C₂-C₆ alkynyl” refers to a straight or branched chain unsaturated hydrocarbon containing 2-6 carbon atoms and at least one triple bond. Examples of a C₂-C₆ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, and 3-hexyne.

“C₃-C₆ alkynyl” refers to a straight or branched chain unsaturated hydrocarbon containing 3-6 carbon atoms and at least one triple bond. Examples of a C₃-C₆ alkynyl group include, but are not limited to, propyne, 1-butyne, 2-butyne, isobutyne, sec-butyne, 1-pentyne, 2-pentyne, isopentyne, 1-hexyne, 2-hexyne, and 3-hexyne.

“C₁-C₆ alkoxy” refers to a straight or branched chain saturated or unsaturated hydrocarbon containing 1-6 carbon atoms and at least one oxygen atom. Examples of a C₁-C₆ alkoxy include, but are not limited to, methoxy, ethoxy, isopropoxy, butoxy, n-pentoxy, isopentoxy, neopentoxy, and hexoxy.

A “5- to 6-membered monocyclic heterocycle” refers to a monocyclic 5- to 6-membered non-aromatic monocyclic cycloalkyl in which 1-4 of the ring carbon atoms have been independently replaced with a N, O or S atom. When a carbon is replaced by N, the N can be substituted with —H, C₁-C₆ alkyl, or acyl. Representative examples of a 5- to 6-membered monocyclic heterocycle group include, but are not limited to, piperidinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl.

Nonlimiting representative “pharmaceutically acceptable salts” include water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

As used herein, “about” means a numeric value having a range of ±10% around the cited value.

A “subject”, as used herein, is a mammal, e.g., a human, mouse, rat, guinea pig, dog, cat, horse, cow, pig, or a non-human primate, such as a monkey, chimpanzee, baboon, or rhesus.

As used herein, “treat”, “treating” or “treatment” refers to administering a therapy in an amount, manner (e.g., schedule of administration), and/or mode (e.g., route of administration), effective to improve a disorder (e.g., an infection described herein) or a symptom thereof, or to prevent or slow the progression of a disorder (e.g., an infection described herein) or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

As used herein, “administered in combination” means that two or more agents are administered to a subject at the same time or within an interval, such that there is overlap of an effect of each agent on the subject. The administrations of the first and second agent can be spaced sufficiently close together such that a combinatorial effect, e.g., a synergistic effect, is achieved. The interval can be an interval of hours, days or weeks. The agents can be concurrently bioavailable, e.g., detectable, in the subject. For example, at least one administration of one of the agents, e.g., an antifungal agent, can be made while the other agent, e.g., a compound described herein, is still present at a therapeutic level in the subject. The subject may have had a response that did not meet a predetermined threshold. For example, the subject may have had a failed or incomplete response, e.g., a failed or incomplete clinical response to the antifungal agent. An antifungal agent and a compound described herein may be formulated for separate administration or may be formulated for administration together.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

The following figures are presented for the purpose of illustration only, and are not intended to be limiting.

### BRIEF DESCRIPTION OF THE DRAWINGS

FIG. 1 is a diagrammatic representation of the properties of an idealized antibiotic agent.

FIG. 2A is a diagrammatic representation of a prodrug screening strategy in which a candidate prodrug is used to contact a microbial cell having reduced (or no) activity of a prodrug activating enzyme.

FIG. 2B is a diagrammatic representation of a prodrug screening strategy in which a candidate prodrug is used to contact a microbial wild type (control) strain.

FIG. 3A is a graphical representation of the level of killing of log-growth, stationary phase and biofilm-phase P. aeruginosa by the bactericidal antibiotic, carbenicillin.

FIG. 3B is a graphical representation of the level of killing of log-growth, stationary phase and biofilm-phase P. aeruginosa by the bactericidal antibiotic, ofloxacin.

FIG. 3C is a graphical representation of the level of killing of log-growth, stationary phase and biofilm-phase P. aeruginosa by the bactericidal antibiotic, tobramycin.

FIG. 3D is a graphical representation of the level of killing of log-growth, stationary phase and biofilm-phase P. aeruginosa by peracetic acid.

FIG. 4 is a diagrammatic representation of the biology of a relapsing biofilm infection.

FIG. 5A is a diagrammatic representation of the reporter system used to separate persisters from growing cells.

FIG. 5B is a graphical representation of two populations that were detected using forward light-scatter, one that fluoresced brightly (R3), and another that did not (R4).

FIG. 5C are photographical representations of microscopic images of the sorted populations visualized by epifluorescent microscopy (bar, 5 μm) using phase contrast or green fluorescence.

FIG. 5D is a graphical representation of the survival of cells sorted as described in FIG. 5B and treated with ofloxacin (5 μg/ml) for three hours and then diluted and spotted onto LB agar plates for colony counts.

FIG. 6 is a representation of a heatmap of selected genes expressed in E. coli persister cells.

FIG. 7 is a graphical representation of the properties of a multidrug tolerance of E. coli expressing HipA.

FIG. 8 is a graphical representation of the effects of toxin deletion on persister formation in E. coli.

FIG. 9 is a diagrammatic representation of a model of multidrug tolerance.

FIG. 10 is a graphical representation of the dose-dependent killing of E. coli by metronidazole. Stationary phase cells grown in LB medium in the presence of 1 mM IPTG under anaerobic conditions were challenged for 6 hrs with increasing concentrations of metronidazole and then plated on LB agar plates.

FIG. 11 is a listing of the chemical structure and source of prodrug antibiotic compounds identified by a first antibiotic screen.

FIG. 12 is a listing of the chemical structure and source of compounds displaying direct activity identified by a first antibiotic screen.

FIG. 13 is a listing of the chemical structure and source of compounds displaying direct activity identified by a second antibiotic screen.

FIG. 14 is a listing of the chemical structure and source of prodrug antibiotic compounds identified by a second antibiotic screen.

FIG. 15 is a listing of the chemical structure and source of antibiotic compounds identified by a second antibiotic screen.

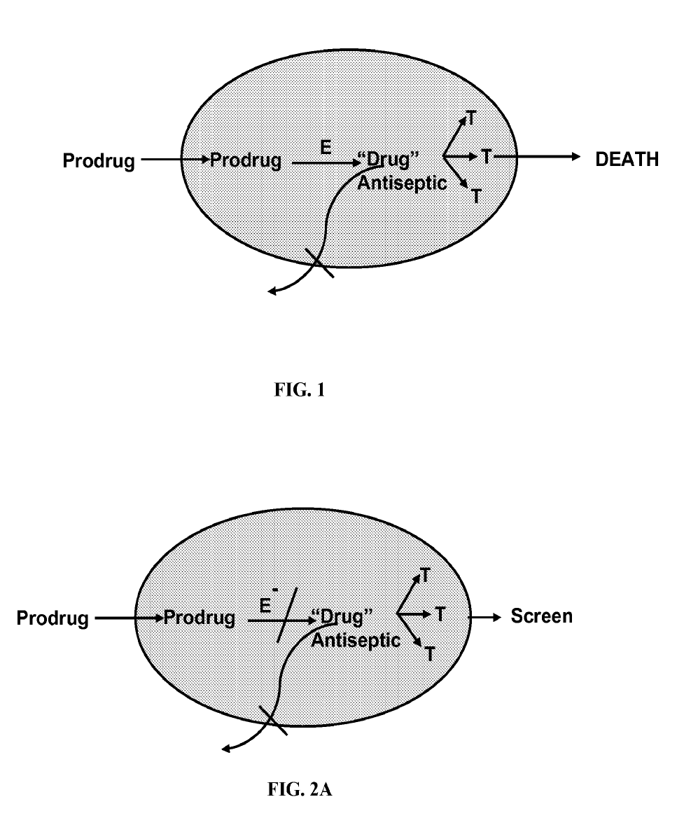

### DETAILED DESCRIPTION OF THE INVENTION

This disclosure relates, in part, to compounds that can function as broad-spectrum antibiotics, sterilizing antibiotics, and/or broad-spectrum sterilizing antibiotics. Some of these compounds are direct inhibitors, while others are prodrugs that can be converted into reactive molecules inside a cell of an organism. The activated prodrug can then bind to its targets and is irreversibly trapped inside the cell. The activated prodrug is able to bypass efflux by MDR pumps and thus has a broad spectrum of activity. Furthermore, because of its non-specific reactivity, the activated prodrug is able to kill dormant persister cells, leading to a complete sterilization of an infection.

### Theoretical Considerations in Antibiotic Prodrug Design

A model antibiotic prodrug is a benign compound that enters into a microbial cell, and is converted by a microbial enzyme into an active, antiseptic-type molecule. This active molecule is more hydrophilic than the prodrug and does not diffuse out of the cell. By the same token, the drug is not a substrate for MDRs that efflux hydrophobic compounds largely based on polarity. The active molecule binds covalently and non-specifically to one or more “targets” within the cell including, but are not limited to, proteins, peptides, cofactors, DNA and the membrane. The active molecule kills both growing and dormant cells.

An ability to kill cells, rather than simply inhibit growth, is required for tuberculosis drugs. The only prodrug with a fairly broad spectrum is metronidazole, which is converted into an active form in bacterial cells under anaerobic conditions and acts specifically against anaerobic species. Accordingly, there is still no single broad-spectrum prodrug antibiotic available.

### Screens to Identify Prodrugs and Direct Inhibitors

The screens described herein are useful for identifying prodrug compounds that are converted inside cells of a pathogen into reactive antiseptic molecules that can kill the pathogen and sterilize the infection caused by the pathogen. The screens described herein also are useful for identifying compounds with direct inhibitory activity. The rationale is to screen compounds against strains differentially expressing an enzyme capable of activating a prodrug into an active compound. A strain overproducing an activating enzyme is more susceptible to a prodrug than the wild type, whereas a strain with a suppressed activating enzyme is more resistant than the wild type. The screens described herein are a departure from traditional approaches based on disabling a particular protein target. A combination of genomics with high throughput screening (HTS) makes this a straightforward approach. Genomics provides candidate enzymes that can activate prodrugs, and a rational screening design enables efficient identification and validation of hits. Conventional whole cell screens suffer from a high background of non-specifically acting compounds such as membrane-acting or DNA-damaging agents. A feature of the screens described herein is the ability to distinguish prodrugs from compounds with direct inhibitory activity, since these will have similar activity against the wild type and strains differentially expressing a prodrug activating enzyme.

a. Screen Using Strains Mutated in an Activating Enzyme

This screen is based on using a microorganism having a mutation in one or more genes. Among these mutants are one or more microorganisms that have mutations in a gene encoding an enzyme that activates a prodrug. The mutation includes, but is not limited to, a loss-of-function mutation, a null mutation, a conditional mutation, or a conditional mutation which is a temperature-sensitive mutation. The mutation may be in an essential gene(s). In ceratin cases, the mutation is in an essential gene in vivo.

In one nonlimiting example, the screen involves contacting a microorganism that is mutant in one or more genes with a candidate compound. The screen involves comparing the level of growth of the mutant microorganism in the presence of the candidate compound to the level of growth of a wild type microorganism in the presence of the candidate antibiotic compound. A greater level of growth of the mutant microorganism in the presence of the candidate compound than the level of growth of the wild-type microorganism in the presence of the candidate compound is indicative of a prodrug activity of the candidate compound. The level of growth of the mutant and wild type organisms can be determined by any method known in the art. In some embodiments, the level of growth is determined in a liquid growth medium. In other embodiments, the level of growth is determined in a plate assay.

The contacting step may be performed with a plurality of mutant microorganisms that are each mutant in different genes but otherwise isogenic. These mutant microbial strains may be mixed together and the resulting suspension can then be used to contact a candidate compound. The suspension may be dispensed into wells of a microtiter plate for screening with the candidate compound. Growth of cells within a suspension of mutant microorganisms contacted with a candidate compound, but not in a suspension of wild type cells contacted with a candidate compound, indicates a prodrug hit. If growth occurs in the suspension of mutants, it is because at least one mutant is mutated in a gene encoding a protein that is necessary to convert the candidate compound into an active drug. Because the prodrug activating enzyme is absent, the prodrug is not converted into its active form and does not kill the cell.

In some cases, resistance may develop against compounds identified by the screen due to null mutations in non-essential activating enzymes. Accordingly, it may be useful to identify prodrug activating enzymes that are essential in vivo. In some cases, the screen identifies prodrug activating enzymes that are essential in vivo (i.e., essential in the organism that the pathogen infects) and therefore not subject to rapid resistance development. In vivo essentiality of a gene of a pathogen within the organism it infects may be determined by any method known in the art. For example, in vivo essentiality of an E. coli gene can be determined by infecting mice with E. coli O157 and following the rate of clearance of knockout mutants: increased clearance indicates essentiality of the gene in vivo.

In certain nonlimiting examples, a secondary screen may then be performed with this prodrug hit compound against each strain of the mutant microbial population dispensed in individual wells, to identify the mutant lacking an activating enzyme for the prodrug. A prodrug has higher detectable activity against a strain expressing an activating enzyme and lower detectable activity against a strain attenuated in this enzyme. This discriminates the prodrug from other compounds and serves to validate the hits.

b. Screen Using Bacterial Strains Having Diminished Expression of Enzymes

In this version of the screen to identify a prodrug compound, a gene of the microorganism is repressed. The method involves contacting a microorganism with a candidate compound while one or more of its genes are repressed. In certain embodiments the repressed gene is a gene encoding a prodrug-activating enzyme. The gene's activity may be repressed using an agent including, but not limited to, antisense oligonucleotides, ribozymes, small interfering RNAs, and aptamers. Methods of making antisense oligonucleotides, ribozymes, small interfering RNAs, and aptamers are well known in the art. The gene's activity may also be repressed using temperature sensitive mutations or by regulating expression of the gene, or an activator or repressor of the gene, through an inducible promoter. The gene may be an essential gene in vivo. The level of growth of the gene-repressed microorganism in the presence of the candidate antibiotic compound is compared to the level of growth of the same microorganism in which the one or more genes of the organism is not repressed. A detectably greater level of growth of the gene-repressed microorganism in the presence of the compound than the level of growth of the non gene-repressed microorganism in the presence of the compound is indicative of a prodrug antibiotic activity of the candidate compound. In some screens, the step of contacting the gene-repressed microorganism with the candidate antibiotic compound comprises simultaneously contacting a plurality of distinct gene-repressed microorganisms that are repressed in distinct genes but otherwise isogenic.

For example, one or more genes of the microorganism may be repressed using antisense technology. The antisense molecule for use in this screen may be produced by a partial or complete cDNA cloned behind a promoter in the antisense orientation. In the antisense RNA approach to the screen, a set of E. coli strains with diminished expression of essential enzymes are constructed and used to screen for prodrugs as described above.

c. Screen Using Strains Overexpressing Prodrug Activating Enzymes

This version of the screen for prodrug compounds is based on overexpression of a prodrug activating enzyme in microbial cells. The rationale of the screen is that a microbial cell overexpressing a prodrug activating enzyme is detectably more susceptible to a prodrug than the wild type microbe. In one example with E. coli overexpressing NfnB, the activating enzyme for metronidazole, the overexpression strain showed greater than 50-fold sensitivity as compared to the wild type control (see, Example 4). Metronidazole completely “sterilized” the population of NfnB overexpressing cells—i.e., it eradicated the NfnB overexpressing cells. This is the first observation of sterilization for an antibiotic. This finding also suggests that finding a prodrug with a better fit to its activating enzyme produces a better therapeutic.

In a nonlimiting example, a set of strains from a library overexpressing conserved essential genes coding for potential prodrug-activating enzymes is used for screen development. In order to validate the functionality of the overexpressed recombinant protein, chromosomal disruptions of the gene are created. An ability to make a knockout validates the functional expression of the recombinant protein, and such a strain becomes part of the screening set. In one embodiment, the enzymes share homology to their counterparts in other microorganisms, and do not have close homologs in humans. Each strain is then screened against a candidate compound, and a compound showing higher activity in the overexpressing strain as compared to the wild type is identified as a prodrug hit.

d. Screens Using Multidrug Pump Mutants

This version of the screen for prodrug compounds is based on contacting a microorganism that is mutant or deficient in multidrug pump efflux with a candidate compound. Compounds activated by prodrug activating enzymes convert into reactive molecules that bind to their targets creating an irreversible sink, thereby inhibiting or preventing multidrug resistance efflux of the activated prodrug. The screen involves comparing the growth of the microorganism that is mutant or deficient in multidrug pump efflux in the presence of the candidate antibiotic compound to the level of growth of a wild type microorganism in the presence of the candidate antibiotic compound. It is to be understood that the wild type microorganism is not mutant or deficient in multidrug pump efflux. If the level of growth of the microorganism that is mutant or deficient in multidrug pump efflux in the presence of the candidate compound is about equal to the level of growth of the wild type microorganism in the presence of the compound, the candidate compound is identified as a prodrug compound.

The mutation may be a loss-of-function mutation, a null mutation, or a conditional mutation in a multidrug efflux gene. In some embodiments, the conditional mutation is a temperature-sensitive mutation. If the microorganism is a bacterium such as Escherichia coli or Salmonella typhimurium, non-limiting examples of multidrug efflux genes include AcrA, AcrB, and TolC. If the microorganism is a bacterium such as Staphylococcus aureus, non-limiting examples of multidrug efflux genes include NorA, NorB, and MepA. If the microorganism is a fungus such as Saccharomyces cerevisiae or Candida albicans, non-limiting examples of multidrug efflux genes include Pdr5, Mdr1, Cdr1, Cdr2, Cdr3, and Flu1. In some cases, the microorganism is made deficient in mutidrug efflux by treating the microbial cell with multidrug pump efflux inhibitors. Non-limiting examples of multidrug pump efflux inhibitors include reserpine, rescinnamine, verapamil, MC207-110, INF 55, INF 271, and PH-Arg-β-naphthylamide.

In all of the screens described above, the microorganism includes, but is not limited to, bacteria, protozoa, and fungi. Any bacterium, protozoan, or fungus may be used in the screens. Examples of bacteria for use in the screens include, but are not limited to, Escherichia coli, Salmonella typhimurium, Staphylococcus aureus, Pseudomonas aeruginosa, Hemophilus influenza, Mycobacterium tuberculosis, and Enterococcus faecalis. Examples of fungi for use in the screens include, but are not limited to, Saccharomyces cerevisiae, Candida albicans.

In all of the screens described above, the compounds identified in the screens can be used to inhibit, reduce, prevent growth of, and/or kill a pathogenic organism. In certain embodiments, the pathogenic organism is a bacterium, a protozoan, a fungus, or a helminth. In some embodiments, the bacteria belong to various Gram-positive and Gram-negative bacteria strains including, but not limited to, Bacillus, Burkholderia, Enterobacter, Escherichia, Helicobacter, Klebsiella, Mycobacterium, Neisseria, Pseudomonas, Staphylococcus, Streptococcus, and Yersinia including drug resistant strains thereof. Non-limiting examples of bacterial pathogenic organisms that can be inhibited or killed by the compounds identified by the screens described herein include Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Hemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, and Yersinia pestis. Non-limiting examples of pathogenic fungal organisms that can be inhibited or killed by compounds identified by the screens described herein include Candida albicans, Candida glabrata, Aspergillus niger, Aspergillus fumigatus, Cryptococcus neoformans, and Pneumocystis carinii. Non-limiting examples of pathogenic protozoan organisms that can be inhibited or killed by compounds identified by the screens described herein include Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, and Microsporidia. Non-limiting examples of helminthic pathogenic organisms that can be inhibited or killed by compounds identified by the screens described herein include flatworms (flukes and tapeworms) and roundworms.

In all of the screens described above, any candidate compound can be assayed. In some embodiments, a candidate compound library is used. Nonlimiting examples of candidate compound libraries include The Compound Library of the New England Regional Center of Excellence for Biodefense and Emergine Infectious Diseases, The Compound Library of the National Institutes of Health Molecular Library Screening Center, The ChemBridge Library, the ChemDiv Library, and the MayBridge Library.

### Biofilms and Persisters

Multidrug tolerance of pathogens is in large part the result of the entry of microbial cells into a dormant state. Such dormant cells are likely responsible for latent (chronic) diseases such as, but not limited to, tuberculosis, syphilis, and Lyme disease, which have thus far been suppressed by known antimicrobials, but not eradicated. Because of the importance of developing therapeutics that are capable of killing these dormant cells and thus eradicating infection, the screens described above can be adapted/modified to identify compounds that have a sterilizing ability against biofilms and persister cells.

Biofilms are bacterial or yeast communities that settle and proliferate on surfaces and are covered by an exopolymer matrix. They are slow-growing and many are in the stationary phase of growth. They can be formed, by most, if not all pathogens. According to the CDC, 65% of all infections in the United States are caused by biofilms that can be formed by common pathogens such as E. coli, P. aeruginosa, S. aureus, E. faecalis, and S. epidermidis. Infections ascribed to biofilms include: childhood middle ear infection and gingivitis; UTI; and infections of indwelling devices such as catheters, heart valves, and orthopedic devices. Biofilm infections also occur in patients with cystic fibrosis. Biofilm infections are highly recalcitrant to antibiotic treatment and adequate therapy against these infections is lacking. While antibiotic treatment will kill most biofilm and planktonic cells, the antibiotics do not kill persisters. The biofilm exopolymer matrix protects against immune cells and persisters that are contained in the biofilm can survive both the onslaught of the antibiotic treatment and the immune system. When antibiotic levels decrease, these persisters can repopulate the biofilm, which will shed off new planktonic cells, producing the relapsing biofilm infection.

Persisters are dormant cells that are tolerant of multiple antibiotics. Bactericidal antibiotics kill cells not by inhibiting its cellular target, but rather by corrupting the target to create a toxic product. For example, aminoglycoside antibiotics kill the cell by interrupting translation, which produces misfolded toxic peptides. Beta-lactam antibiotics, such as penicillin, inhibit peptidoglycan synthesis, which activates autolysin enzymes present in the cell wall leading to digestion of the peptidoglycans and cell death. Fluoroquinolones inhibit the ligase step of DNA gyrase and topoisomerase, without affecting its nicking activity, thereby converting these enzymes into endonucleases. The ability of persister cells to survive killing by antibiotics without expressing or using resistance mechanisms (i.e., tolerance) is mediated by preventing target corruption by a bactericidal agent through the blocking of antibiotic targets. If persisters are dormant and have minimal cell wall synthesis, translation, or topoisomerase activity, then the antibiotics will bind to, but will be unable to corrupt, the function of their targets. In this way, tolerance could enable resistance of persister cells to killing by antibiotics, but at the price of non-proliferation. The simplest method to form a persister cell is through the overproduction of proteins that are toxic to the cell and inhibit growth.

Given the prominent role of tolerance to antibiotics in infectious disease, the need for compounds that can eradicate persisters is clear. The compounds can be tested for their ability to eradicate stationary populations of the pathogen.

### The Compounds of Formulae (I) and (II) and Compounds 8, 9, 10, and 11

The present disclosure features compounds of the Formula I below

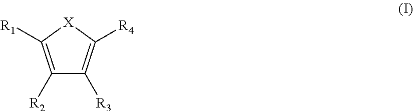

and pharmaceutically acceptable salts, hydrates, and solvates thereof, wherein R₁-R4, Ra and X are as defined above for the compounds of Formula (I) and wherein the compounds are not 5-bromo-N-phenylthiophene-2-carboxamide, 1,3,5-triazatricyclo[3.3.3.1.1]decan-7-amine, N-[(5-nitro-2-thienyl)methylene], 4-chloro-2-methyl-N-((5-nitrofuran-2-yl)methylene)aniline, 4-bromo-2-(2-nitrovinyl)thiophene, 3-(2-nitrovinyl)thiophene, (E)-3-ethyl-54(4-ethyl-3,5-dimethyl-2H-pyrrol-2-ylidene)methyl)-2,4-dimethyl-1H-pyrrole, (4,5,6,7-tetrahydrobenzo[b]thiophen-3-yl)methyl carbamimidothioate, or 5-nitrofuran-2-carboxamide.

In some embodiments, X is O. In other embodiments, X is NH. In still other embodiments, X is S.

In some embodiments, R₁ is H. In other embodiments, R₁ is NO₂.

In some embodiments, R₂ is Br.

In some embodiments, R₃ is —CH═CHNO₂. In other embodiments, R₃ is —CH₂SC(NH)NH₂.

In some embodiments, R₄ is

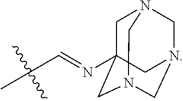

In other embodiments, R₄ is

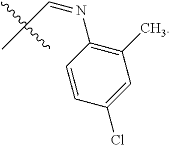

In still other embodiments, R₄ is

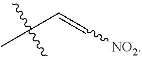

In yet other embodiments, R₄ is

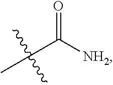

The disclosure also provides features compounds of Formula II below

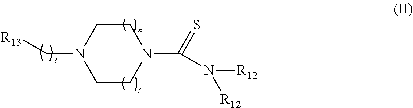

and pharmaceutically acceptable salts, hydrates, and solvates thereof, wherein R₁₂, R₁₃, n, p, and q are as defined above for compounds of formula (II) and wherein the compounds are not 3-(3-chlorobenzyl)-N-(3-chlorophenyl)tetrahydropyrimidine-1(2H)-carbothioamide, 7-(4-(benzo[d][1,3]dioxol-5-ylcarbamothioyl)piperazin-1-yl)-1-ethyl-6-fluoro-4-oxo-1,4-dihydroquinoline-3-carboxylic acid, or 1-ethyl-6-fluoro-4-oxo-7-(4-(3-phenyl isoxazole-4-carbonylcarbamothioyl)piperazin-1-yl)-1,4-dihydroquinoline-3-carboxylic acid.

In some embodiments, R₁₂ is

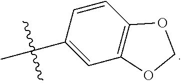

In other embodiments, R₁₂ is

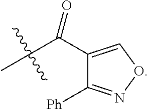

In other embodiments, R₁₃ is

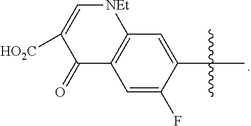

In some embodiments, n is 1. In some embodiments, p is 1. In some embodiments, q is 1.

The disclosure also relates to analogs of Compound 6

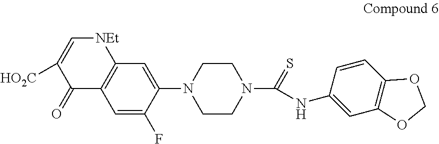

and pharmaceutically acceptable salts, hydrates, and solvates thereof, wherein

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₂CH₃, attached to nitrogen or oxygen, can be substituted with any one of the following substituents: C₁₋₆ alkyl; C₂₋₆ alkenyl; C₂₋₆ alkynyl; —C(O)C₁₋₆ alkyl; —C(O)OC₁₋₆ alkyl; C₃₋₆ cycloalkyl; C₃₋₆ cycloalkyl-C₁₋₃ alkyl; alkylaryl; aryl; arylalkyl; and heteroaryl;

—H attached to oxygen can be substituted with any one of the following substituents: C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl; and

the analog is not 7-(4-(benzo[d][1,3]dioxol-5-ylcarbamothioyl)piperazin-1-yl)-1-ethyl-6-fluoro-4-oxo-1,4-dihydroquinoline-3-carboxylic acid.

The invention also relates to analogs of Compound 7

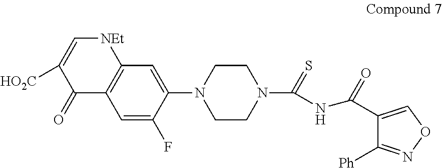

and pharmaceutically acceptable salts, hydrates, and solvates thereof, wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₂CH₃, attached to nitrogen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

—H attached to oxygen can be substituted with any one of the following substituents: C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl; and

the phenyl attached to the isoxazole can be replaced with C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl; and wherein the analog is not 1-ethyl-6-fluoro-4-oxo-7-(4-(3-phenylisoxazole-4-carbonylcarbamothioyl)piperazin-1-yl)-1,4-dihydroquinoline-3-carboxylic acid.

The disclosure also relates to analogs of Compound 8

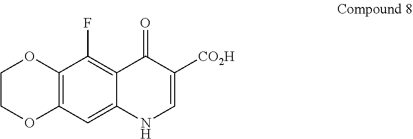

and pharmaceutically acceptable salts, hydrates, and solvates thereof, wherein:

any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and

the analog is not 2,3,6,9-tetrahydro-9-oxo-1,4-dioxino[2,3-g]quinoline-8-carboxylic acid.

The disclosure also relates to analogs of Compound 9

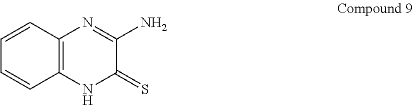

and pharmaceutically acceptable salts, hydrates, and solvates thereof, wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the C(S) can be substituted with C(O); and

the analog is not 3-aminoquinoxaline-2(1H)-thione.

The disclosure also relates to analogs of Compound 10

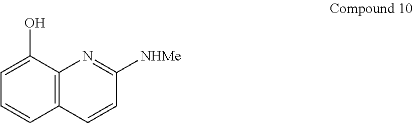

and pharmaceutically acceptable salts, hydrates, and solvates thereof, wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;

any one or more of —H and —CH₃ attached to nitrogen or oxygen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and

the analog is not 2-(methylamino)quinolin-8-ol.

The disclosure also relates to analogs of Compound 11

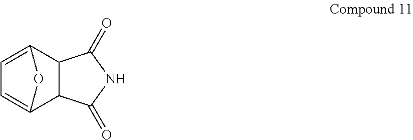

and pharmaceutically acceptable salts, hydrates, and solvates thereof, wherein:

any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and

any one or more of —H and —CH₃ attached to nitrogen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and wherein the analog is not 4,7-epoxy-1H-isoindole-1,3(2H)-dione.

### Methods of Making Compounds

The compounds described herein and pharmaceutically acceptable thereof can be prepared using a variety of methods starting from commercially available compounds, known compounds, or compounds prepared by known methods.

For example, compounds 1-11 are commercially available from Chembridge Corp. (San Diego, U.S.A.), and Maybridge, plc, (Cornwall, UK). Those of skill in the art employing known organic chemical synthetic methods can synthesize any of the compounds described herein. For example, treatment of an alkyl or aryl group with lithium, followed by reaction with an electrophile, will substitute an —H with C₁₋₆ alkyl, C₂₋₆ alkenyl, C₂₋₆ alkynyl, —C(O)C₁₋₆alkyl, —C(O)OC₁₋₆alkyl, C₃₋₆ cycloalkyl, C₃₋₆ cycloalkyl-C₁₋₃ alkyl, alkylaryl, aryl, arylalkyl, heteroaryl, or heteroarylalkyl (see e.g. Carey and Sundberg, Advanced Organic Chemistry, 4^{th} ed., Plenum Publishers, Boston, pp. 444, 693, 714, 885 (2001). Reaction of aryl groups with strong acids such a HNO₃, HCN, or HCl will substitute an H for NO₂, CN, or Cl, respectively (see Carey and Sundberg, p. 191). Further acid hydrolysis of the CN moiety will liberate a carboxylic acid moiety that can be further derivatized by esterification, reduction or amination. (see Patrick, Organic Chemistry, Springer Verlag, NY, p. 220-221 (2000) (Patrick). Alkylation and acylation of amines will change the substitution on the nitrogens of the compounds (see e.g. Patrick, p. 299).

### Methods of Using Compounds

The compounds described herein exhibit the ability to kill bacteria, fungi, protozoa, and helminth and, therefore, can be utilized in order to treat or prevent infections by these organisms. Thus, the compounds described herein are effective in the treatment of any disease or symptom of a disease caused by or resulting from an infection by a bacterium, a protozoan, a fungus, and/or a helminth. In particular, the compounds described herein possess cell growth inhibiting effects and are effective in treating, for example, upper respiratory tract diseases; infections of catheters; infections of orthopedic prostheses; Urinary Tract Infections (UTI); gastrointestinal infections; heart valves infections; endocarditis; skin infections; chronic wounds; and cystic fibrosis.

Therapeutic Administration

The route and/or mode of administration of a compound described herein can vary depending upon the desired results. Dosage regimens can be adjusted to provide the desired response, e.g., a therapeutic response.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration can result in release of a potentiator and/or an antifungal agent described herein into the bloodstream. The mode of administration is left to the discretion of the practitioner.

In some instances, a compound described herein can be administered locally. This can be achieved, for example, by kcal infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes, or fibers.

In some situations, a compound described herein can be introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated, e.g., by an intraventricular catheter, for example, attached to a reservoir, such as an Ommaya reservoir.

This disclosure also features a device for administering a compound described herein. The device can include, e.g., one or more housings for storing pharmaceutical compositions, and can be configured to deliver unit doses of a compound described herein.

Pulmonary administration can also be employed, e.g., by use of an inhaler or nebulizer, and formulation with an aerosolizing agent, or via perfusion in a fluorocarbon or synthetic pulmonary surfactant.

In some instances, a compound described herein can be delivered in a vesicle, in particular a liposome (see Langer, Science 249:1527-1533 (1990) and Treat et al., Liposomes in the Therapy of Infectious Disease and Cancer pp. 317-327 and pp. 353-365 (1989)).

In yet other situations, a compound described herein can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet other situations, a controlled- or sustained-release system can be placed in proximity of a target of compound described herein, e.g., the reproductive organs, reducing the dose to a fraction of the systemic dose.

A compound described herein can be formulated as a pharmaceutical composition that includes a suitable amount of a physiologically acceptable excipient (see, e.g., Remington's Pharmaceutical Sciences, pp. 1447-1676 (Alfonso R. Gennaro, ed., 19th ed. 1995)). Such physiologically acceptable excipients can be, e.g., liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to an animal. The physiologically acceptable excipient should be stable under the conditions of manufacture and storage and should be preserved against the contaminating action of microorganisms. Water is a particularly useful excipient when a compound described herein is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable physiologically acceptable excipients are described in Remington's, ibid. The pharmaceutical compositions, if desired, can also contain minor amounts of wetting or emulsifying agents, or pH buffering agents.

Liquid carriers can be used in preparing solutions, suspensions, emulsions, syrups, and elixirs. A compound described herein can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives described herein, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. The liquid carriers can be in sterile liquid form for administration. The liquid carrier for pressurized compositions can be halogenated hydrocarbon or other pharmaceutically acceptable propellant.

A compound described herein can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule.

In some instances, a compound described herein is formulated in accordance with routine procedures as a composition adapted for oral administration to humans. Compositions for oral delivery can be in the form of, e.g., tablets, lozenges, buccal forms, troches, aqueous or oily suspensions or solutions, granules, powders, emulsions, capsules, syrups, or elixirs. Orally administered compositions can contain one or more additional agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with a finely divided compound described herein. In tablets, a compound described herein can be mixed with a carrier having compression properties in suitable proportions and compacted in the shape and size desired. The powders and tablets can contain up to about 99% of a potentiator and/or an antifungal agent described herein.

Capsules can contain mixtures of a compound described herein with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, a compound described herein can be coated to delay disintegration and absorption in the gastrointestinal tract, thereby providing a sustained action over an extended period of time. Selectively permeable membranes surrounding an osmotically active driving a compound described herein can also be suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In some situations, the excipients are of pharmaceutical grade.

In other instances, a compound described herein can be formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. The ingredients can be supplied either separately or mixed together in unit dosage form, for example, as a dry lyophilized powder or water-free concentrate in a hermetically sealed container such as an ampule or sachette indicating the quantity of active agent. Where a compound described herein is administered by infusion, it can be dispensed, for example, with an infusion bottle containing sterile pharmaceutical grade water or saline. Where a compound described herein is administered by injection, an ampule of sterile water for injection or saline can be provided so that the ingredients can be mixed prior to administration.

In other circumstances, a compound described herein can be administered across the surface of the body and the inner linings of the bodily passages, including epithelial and mucosal tissues. Such administrations can be carried out using a compound described herein in lotions, creams, foams, patches, suspensions, solutions, and suppositories (e.g., rectal or vaginal). In some instances, a transdermal patch can be used that contains a compound described herein and a carrier that is inert to the compound described herein, is non-toxic to the skin, and that allows delivery of the agent for systemic absorption into the blood stream via the skin. The carrier can take any number of forms such as creams or ointments, pastes, gels, or occlusive devices. The creams or ointments can be viscous liquid or semisolid emulsions of either the oil-in-water or water-in-oil type. Pastes of absorptive powders dispersed in petroleum or hydrophilic petroleum containing a compound described herein can also be used. A variety of occlusive devices can be used to release a compound described herein into the blood stream, such as a semi-permeable membrane covering a reservoir containing the compound described herein with or without a carrier, or a matrix containing the compound described herein.

A compound described herein can be administered rectally or vaginally in the form of a conventional suppository. Suppository formulations can be made using methods known to those in the art from traditional materials, including cocoa butter, with or without the addition of waxes to alter the suppository's melting point, and glycerin. Water-soluble suppository bases, such as polyethylene glycols of various molecular weights, can also be used.

The amount of a compound described herein that is effective for treating an infection can be determined using standard clinical techniques known to those will skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. For example, the dose of a compound described herein can each range from about 0.001 mg/kg to about 250 mg/kg of body weight per day, from about 1 mg/kg to about 250 mg/kg body weight per day, from about 1 mg/kg to about 50 mg/kg body weight per day, or from about 1 mg/kg to about 20 mg/kg of body weight per day. Equivalent dosages can be administered over various time periods including, but not limited to, about every 2 hrs, about every 6 hrs, about every 8 hrs, about every 12 hrs, about every 24 hrs, about every 36 hrs, about every 48 hrs, about every 72 hrs, about every week, about every two weeks, about every three weeks, about every month, and about every two months. The number and frequency of dosages corresponding to a completed course of therapy can be determined according to the judgment of a health-care practitioner.

In some instances, a pharmaceutical composition described herein is in unit dosage form as described above, e.g., as a tablet, capsule, powder, solution, suspension, emulsion, granule, or suppository. In such form, the pharmaceutical composition can be sub-divided into unit doses containing appropriate quantities of a potentiator and/or an antifungal agent described herein. The unit dosage form can be a packaged pharmaceutical composition, for example, packeted powders, vials, ampoules, pre-filled syringes or sachets containing liquids. The unit dosage form can be, for example, a capsule or tablet itself, or it can be the appropriate number of any such compositions in package form. Such unit dosage form can contain from about 1 mg/kg to about 250 mg/kg, and can be given in a single dose or in two or more divided doses.

The invention is further described in the following examples, which do not limit the scope of the invention described in the claims.

### EXAMPLES

### Example 1

### Pilot Screens With Mutant Pools

Pilot screens were performed to identify compounds that have a lower activity against a bacterial strain deleted in an activating enzyme as compared to the wild type.

A complete, ordered E. coli K12 knockout library of 4320 genes and predicted ORFs (the Keio library) was used (Baba et al. (2006). Mol. Systems Biol. 2:2006.0008). The knockouts were constructed using the Wanner method with a kanamycin cassette replacing the ORFs (Datsenko et al. (2000) Proc. Natl. Acad. Sci. USA 97:6640-6645). All strains of this library were combined in a mix BacPool1) for screening. This permitted screening of the library against all strains simultaneously, instead of screening a full industry-size library against each of the individual E. coli 4×10³ knockout strains.

The library mix was prepared by first culturing all strains overnight in LB medium, and then adding equal aliquots into a tube. This material was then mixed, dispensed in vials and stored at −80° C. For the screen, one vial was thawed, diluted to 10⁵ cells/ml in LB, and dispensed into 384 well microtiter plates. The compounds were added from DMSO stocks at a final concentration of 46 μg/ml. The same compounds were added to plates containing the wild type isogenic control. Screening was performed at the Harvard NSRB/NERCE screening facility that host a collection of 150,000 compounds (see Tables 1A and 1B). All screens at NSRB/NERCE were performed in duplicate, which strongly decreases the rate of false positives and negatives. For each tested molecule there are three possible scenarios when scoring for growth/no growth:

Growth in both the BacPool and K12 wells: indicates lack of antimicrobial activity;

Growth inhibition in both the BacPool and K12 wells: a possible antibiotic (direct activity) or generally toxic compound is present, but is not a prodrug; or

Growth of a BacPool well and no growth of the K12 well: a prodrug hit.

After an overnight incubation of plates at 37° C., the plates were read at OD₆₀₀. Compounds that had no effect on the growth of the mix and inhibited the growth of the wild type were recorded as hits. The wells with growth indicated a prodrug hit that is not converted into a drug by a particular deletion mutant in the mix. The mutant strain is then identified in a secondary screen (see Example 2).

TABLE 1A
The NSRB/NERCE Library

| | Number of | ICCB Plate |
|---|---|---|
| Library Name | Compounds | Numbers |
| Biomol-TimTec 1 | 8,518 | 1534-1558 |
| Bionet 1 | 4,800 | 0568-0582 |
| Bionet 2 | 1,700 | 1364-1368 |
| CEREP | 4,800 | 0526-0540 |
| ChemBridge Microformat | 50,000 | 0686-0842 |
| ChemDiv1 (Combilab and | 28,864 | 0587-0668 |
| International) | | |
| ChemDiv 2 | 8,560 | 1369-1393 |
| ChemDiv 3 | 16,544 | 1473-1519 |
| ChemDiv-Anti-Mitotic | 1,254 | 1157-1160 |
| Collection | | |
| Enamine 1 | 6,004 | 1394-1411 |
| I.F. Lab 1 | 6,543 | 1412-1430 |
| I.F. Lab 2 | 292 | 1459 |
| Maybridge 1 | 8,800 | 0542-0566 |
| Maybridge 2 | 704 | 1303-1304 |
| Maybridge 3 | 7,639 | 1431-1452 |
| Maybridge 4 | 4,576 | 1521-1533 |
| Peakdale 1 | 2,816 | 0518-0525 |
| Peakdale 2 | 352 | 1305 |
| Mixed Commercial Plate 1 | 352 | 0541 |
| Mixed Commercial Plate 2 | 320 | 0567 |
| Mixed Commercial Plate 3 | 251 | 0669 |
| Mixed Commercial Plate 4 | 331 | 1306 |
| Mixed Commercial Plate 5 | 268 | 1520 |

TABLE 1B
Known Bioactives Collections

| | Number of | ICCB Plate |
|---|---|---|
| Library Name | Compounds | Numbers |
| NINDS Custom Collection | 1,040 | 0500-0503 |
| SpecPlus Collection | 960 | 1081-1083 |
| BIOMOL ICCB Known Bioactives | 480 | 1361-1362 |

The general procedure to establish the Z′-factor was tested. Since the output of the screening is a typical growth/no growth assay, this was performed by comparing growth in control wells to those containing a model antimicrobial, ciprofloxacin at 30 μg/ml. E. coli K12 were cultured in LB medium, and exponentially-growing cells were dispensed at 10⁶/ml in 384-well microtiter plates, 30 μl/well. Six plates were used in this experiment. Ciprofloxacin was added to half of the wells (3 μl in LB, bringing the final volume to 33 μl). After an overnight incubation at 37 C, the OD₆₀₀ of the plates were read, and the values of each well were used to calculate Z′-factor: Z′=1−(3SD++3SD−)/IAve₊−Ave₋I, where: SD+=positive control standard deviation; SD−=negative control standard deviation; Ave+=positive control average; and Ave−=negative control average). The following table for Z′-factor interpretation was used:

### High-throughput Screening Assay Fitness:

1>Z′>0.9 An excellent assay;
0.9>Z′>0.7 A good assay;
0.7>Z′>0.5 Hit selection will benefit significantly from any improvement; and
0.5=Z′ The absolute minimum recommend for high throughput screening

The Z′-factor for the assay was 0.75, suggesting that pilot screening could be performed. It is important to note that the deviation from perfect results in this screen was due to normal variations in OD reading among wells, rather than to false positives or false negatives. There was no case of substantial growth in a well with ciprofloxacin or lack of growth in a well without an antibiotic.

A first pilot screen of 3000 compounds from the NERCE library was performed in duplicate to reduce variability (screening in duplicate is standard procedure at this facility). The controls were E. coli W3100 cells, which were compared to a pool of 4320 knockout strains from the Keio library (BacPool). The pool was prepared by growing each mutant overnight in microtiter plates in LB at 37° C. and then mixing all of them in equal amounts. The compounds were dispensed at a final concentration of 46 μg/ml in 275 nl volume. The screen produced 3 hits.

In a second pilot screen of 45,000 compounds from the NERCE library were tested and 17 prodrug hits were obtained (i.e., a frequency of 0.037%).

### Example 2

### Secondary Screen

Once the hits are obtained from the screen, they can be verified by retesting the hit compounds against the mix and the wild type. Confirmed hits are examined further. Strains containing the activating enzymes are determined with a secondary screen against individual deletion strains.

In the secondary screen, the deletion strain lacking the activating enzyme is identified by testing against the strains of the knockout library dispensed in individual wells. This will identify the resistant strain lacking an activating enzyme.

### Example 3

### Hit Validation Using Strains Overexpressing Prodrug Activating Enzymes

Next, the hits that verify and have reduced activity compared to wild type or no activity against strains deleted in a known or putative enzyme are validated. The rationale is to test the hit against a strain overexpressing the putative activating enzyme. Strains overexpressing activating enzymes have considerably higher susceptibility to prodrugs. It is important to note that conventional antibiotics that inhibit specific targets have increased activity against strains with diminished expression of the target, and decreased activity if the target is overproduced (Schmid (2001) in Antibiotic Development and Resistance. Hughes and Andersson (eds). New York; Taylor and Francis, pp. 197-208; Sun, D. (2001) in 41st Interscience Conference on Antimicrobial Agents and Chemotherapy Chicago: ASM, pp. 77). A prodrug hit has higher activity against a strain overexpressing an activating enzyme, and lower activity against a strain lacking/diminished in an activating enzyme. This behavior of a prodrug hit is the exact opposite from what one expects from a specific antibiotic, and provides for validation of prodrugs. This validation is facilitated by the availability of the ASKA library of E. coli strains overexpressing all known ORFs, (The University of Nagoya-Saka et al. (2005) DNA Res. 12:63-68). The library contains 4,382 individual E. coli K12 W3110 clones, each carrying a single ORF cloned in an expression vector pCA24N under a pT5/lac promoter. The N-termini carry a his-tag linked to the ORF by a 7 amino acid spacer. The vector carries a CAM resistance marker and a lac^{q} gene for a tight control of IPTG-inducible expression.

The ASKA strains of interest are validated for functional expression of the enzyme. If the protein is not expressed from a given expression vector the ORF is recloned. A strain overexpressing the activating enzyme from the ASKA library is then tested with the hit compound. If the hit has greater activity against this overexpressing strain as compared to the wild type, this indicates a prodrug. The test is performed in a standard broth microdilution assay for MIC determination. Hits with the lowest wild type MIC are then examined.

### Example 4

### Validation of Deletion and Overexpression of Prodrug Activating Enzyme Screens

Known antimicrobial prodrugs were used in order to validate the proposed screen with E. coli strains overexpressing the activating enzymes. Most known prodrugs are specific for M tuberculosis, and the broader-spectrum metronidazole is ineffective against E. coli. Metronidazole was reexamined because its activity may be within a measurable range with an E. coli strain overexpressing an activating enzyme. A number of E. coli activating enzymes that have been developed to activate prodrugs used in cancer chemotherapy were also utilized (Table 2). The approach, known as Gene-Directed Enzyme-Prodrug Therapy (GDEPT), is based on delivering a gene coding for the bacterial activating enzyme into cancer tissue, and then adding a prodrug that converts into a cytotoxic compound in the cell. Dinitroaziridinylbenzamide and dinitrobenzamide convert into active antiseptic-like molecules, and in this regard match the type of compounds that are sought in the screen. Fludarabine and 5-fluorocytosine are analogs of nucleotides that stop cells from growing when incorporated into DNA. Whether these compounds are active against bacteria is unknown.

TABLE 2
Prodrugs Used in Cancer Gene-Directed Enzyme-Prodrug Therapy

| Molecule | Structure | Activating enzyme |
|---|---|---|
| 5-Fluorocytosine | 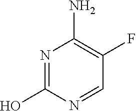 | Cytosine deaminase (CodA)(Mullen et al., 1992; Tiraby et al., 1998) |
| Fludarabine | 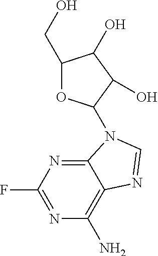 | Purine nucleoside phosphorylase (DeoD)(Huang and Plunkett, 1987) |
| Dinitroaziridinyl- benzamide (CB1954) | 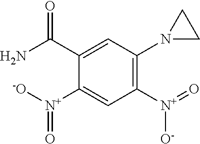 | Nitroreductase (NfnB/NfsA) (Knox et al., 1988; Knox et al., 1992) |
| 3,5-Dinitro- benzamide | 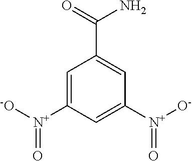 | Nitroreductase (NfnB/NfsA) (Denny, 2003) |

The prodrugs are converted into drugs by the cancer cells expressing the corresponding bacterial enzyme.

Metronidazole is converted into an active drug by the nitrate reductase of H. pylori (van der Wouden et al., Scand. J. Gastroenterol. Suppl. 234:10-14, 2001) and other bacteria. E. coli has two nitrate reductases, NfnB and NfsA. As expected, metronidazole was essentially ineffective against the wild type, with an MIC>500 μg/ml (Table 3). However, metronidazole appeared to be an effective antimicrobial against the strain overexpressing NfnB and NfsA (MIC 8.8 μg/ml). Strains deleted in the enzymes showed even greater resistance than the wild type. The use of deletion strains to validate prodrug candidates. Opposing susceptibilities of an overexpressing versus a deleted strain points to the prodrug nature of a hit compound.

Similarly, significantly increased susceptibilities were observed with dinitroaziridinylbenzamide and dinitrobenzamide against strains overexpressing the corresponding activating enzymes. 5-fluorocytosine showed little activity against any strains tested. Taken together, the results suggest that differential expression of an activation enzyme can be used to develop a specific screen for prodrugs.

TABLE 3
Effect of Overexpression and Deletion of an Activating Enzyme

| | | E. coli K12 | |
|---|---|---|---|
| | | overexpressing | E. coli K12 |
| | E. coli | activating | Δactivating |
| Compound | K12 wt | enzyme | enzyme |
| Metronidazole | 563 | nfnB⁺ 8.8 | nfnB⁻ 1125 |
| | | nfsA⁺ 8.8 | nfsA⁻ 2250 |
| Dinitroaziridinylbenzamide | >200 | nfnB⁺ 3.3 | nfnB⁻ > 200 |
| (CB1954) | | nfsA⁺ 6.3 | nfsA⁻ > 200 |
| 3,5-Dinitrobenzamide | 250 | nfnB⁺ 15.6 | nfnB⁻ > 500 |
| | | nfsA⁺ 7.8 | nfsA⁻ > 500 |
| Fludarabine | >500 | deoD⁺ 125 | deoD⁻ > 500 |
| 5-fluorocytosine | >2500 | codA⁺ 625 | codA⁻ > 2500 |
| Erythromycin | 200 | nfnB⁺ 200 | |
| | | nfsA⁺ 200 | |
| | | deoD⁺ 200 | |
| | | codA⁺ 200 | |

### Example 5

### Prodrug Screening Based on Essential Protein Overexpression

A different modality of the prodrug screen is examined based on the increased sensitivity to prodrugs of a strain overexpressing an activating enzyme as compared to the wild type. For this screen, strains overexpressing enzymes from the ASKA library described above are used. Since each strain has to be screened individually, the number of strains is limited to those that express enzymes that are essential and conserved.

There are approximately 300 essential genes in E. coli (Gerdes et al. (2003) J. Bacteriol. 185:5673-5684), and from this list essential known and putative enzymes were identified (Table 4). Apart from the annotation of an essential protein as an enzyme, two additional significant criteria are used—absence of an obvious homolog in humans; and presence of a homolog in M. tuberculosis. Lack of human homologs increases the chances of finding non-toxic compounds. Conservation among E. coli and M. tuberculosis indicates a generally conserved nature of potential prodrug-activating enzymes, and treatment of tuberculosis is one of the important applications for sterilizing antimicrobial compounds. Using these criteria, a select set of 50 potential prodrug-activating enzymes is obtained.

TABLE 4
Essential Candidate Prodrug Activating Genes

| | | Mtub | Human | | | | |
|---|---|---|---|---|---|---|---|
| Gene | Essential | homolog | homolog | Length | SwissProt | B-name | Annotation |
| AckA | Y | Y | N | 400 | P15046 | b2296 | Acetate kinase (EC |
| | | | | | | | 2.7.2.1) |
| ArgC | Y | Y | N | 334 | P11446 | b3958 | N-acetyl-gamma- |
| | | | | | | | glutamyl-phosphate |
| | | | | | | | reductase (EC 1.2.1.38) |
| asd | Y | Y | N | 367 | P00353 | b3433 | Aspartate-semialdehyde |
| | | | | | | | dehydrogenase (EC |
| | | | | | | | 1.2.1.11) |
| BtuR | Y | Y | N | 196 | P13040 | b1270 | COB(I)alamin |
| | | | | | | | adenosyltransferase (EC |
| | | | | | | | 2.5.1.17) |
| CoaD | Y | Y | N | 159 | P23875 | b3634 | Phosphopantetheine |
| | | | | | | | adenylyltransferase (EC |
| | | | | | | | 2.7.7.3) |
| CysE | Y | Y | N | 273 | P05796 | b3607 | Serine acetyltransferase |
| | | | | | | | (EC 2.3.1.30) |
| DapA | Y | Y | Y/N | 292 | P05640 | b2478 | Dihydrodipicolinate |
| | | | | | | | synthase (EC 4.2.1.52) |
| DapB | Y | Y | N | 273 | P04036 | b0031 | Dihydrodipicolinate |
| | | | | | | | reductase (EC 1.3.1.26) |
| DapD | Y | Y | N | 274 | P03948 | b0166 | Tetrahydrodipicolinate |
| | | | | | | | N-succinyltransferase |
| | | | | | | | (EC 2.3.1.117) |
| DapF | Y | Y | N | 275 | P08885 | b3809 | Diaminopimelate |
| | | | | | | | epimerase (EC 5.1.1.7) |
| DdlB | Y | Y | N | 306 | P07862 | b0092 | D-alanine--D-alanine |
| | | | | | | | ligase B (EC 6.3.2.4) |
| Dxr | Y | Y | N | 398 | P45568 | b0173 | 1-deoxy-D-xylulose 5- |
| | | | | | | | phosphate |
| | | | | | | | reductoisomerase (EC |
| | | | | | | | 1.1.1.267) |
| ElaA | Y | Y | N | 153 | P52077 | b2267 | GTP-binding protein |
| | | | | | | | ElaA |
| FbaA | Y | Y | N | 359 | P11604 | b2925 | Fructose-bisphosphate |
| | | | | | | | aldolase class II (EC |
| | | | | | | | 4.1.2.13) |
| FrlD | Y | Y | N | 261 | P45543 | b3374 | Fructoselysine kinase |
| FtsI | Y | Y | N | 588 | P04286 | b0084 | Peptidoglycan synthetase |
| HemD | Y | Y | N | 246 | P09126 | b3804 | Uroporphyrinogen-III |
| | | | | | | | synthase (EC 4.2.1.75) |
| IspE | Y | Y | N | 283 | P24209 | b1208 | 4-diphosphocytidyl-2-C- |
| | | | | | | | methyl-D-erythritol |
| | | | | | | | kinase (EC 2.7.1.148) |
| IspF | Y | Y | N | 159 | P36663 | b2746 | 2-C-methyl-D-erythritol |
| | | | | | | | 2,4-cyclodiphosphate |
| | | | | | | | synthase (EC 4.6.1.12) |
| IspG | Y | Y | N | 372 | P27433 | b2515 | 1-hydroxy-2-methyl-2- |
| | | | | | | | (E)-butenyl 4- |
| | | | | | | | diphosphate synthase |
| | | | | | | | (gcpE) |
| IspH | Y | Y | N | 316 | P22565 | b0029 | 4-hydroxy-3-methylbut- |
| | | | | | | | 2-enyl diphosphate |
| | | | | | | | reductase |
| Lgt | Y | Y | N | 291 | P37149 | b2828 | Prolipoprotein |
| | | | | | | | diacylglyceryl transferase |
| | | | | | | | (EC 2.4.99.—) |
| MenE | Y | Y | Y/N | 451 | P37353 | b2260 | O-succinylbenzoic acid-- |
| | | | | | | | CoA ligase (EC 6.2.1.26) |
| MesJ | Y | Y | N | 432 | P52097 | b0188 | tRNA(Ile)-lysidine |
| | | | | | | | synthetase |
| MrdA | Y | Y | N | 633 | P08150 | b0635 | Penicillin-binding protein |
| | | | | | | | 2, |
| | | | | | | | transglycosylase/ |
| | | | | | | | transpeptidase |
| Mtn | Y | Y | N | 232 | P24247 | b0159 | MTA/SAH nucleosidase |
| MurC | Y | Y | N | 491 | P17952 | b0091 | UDP-N-acetylmuramate-- |
| | | | | | | | alanine ligase (EC |
| | | | | | | | 6.3.2.8) |
| MurD | Y | Y | N | 438 | P14900 | b0088 | UDP-N- |
| | | | | | | | acetylmuramoylalanine-- |
| | | | | | | | D-glutamate ligase (EC |
| | | | | | | | 6.3.2.9) |
| murE | Y | Y | N | 495 | P22188 | b0085 | UDP-N- |
| | | | | | | | acetylmuramoylalanyl- |
| | | | | | | | D-glutamate--2,6- |
| | | | | | | | diaminopimelate ligase |
| | | | | | | | (EC 6.3.2.13) |
| MurG | Y | Y | N | 355 | P17443 | b0090 | UDP-N- |
| | | | | | | | acetylglucosamine--N- |
| | | | | | | | acetylmuramyl- |
| | | | | | | | (Pentapeptide) |
| | | | | | | | pyrophosphoryl- |
| | | | | | | | undecaprenol N- |
| | | | | | | | acetylglucosamine |
| | | | | | | | transferase (EC 2.4.1.—) |
| MurI | Y | Y | N | 289 | P22634 | b3967 | Glutamate racemase (EC |
| | | | | | | | 5.1.1.3) |
| Pay | Y | Y | N | 196 | P77181 | b1400 | Phenylacetic acid |
| | | | | | | | degradation protein, |
| | | | | | | | predicted acyltansferase |
| PspE | Y | Y | N | 104 | P23857 | b1308 | Rhodanese-related |
| | | | | | | | sulfurtransferase |
| PyrH | Y | Y | N | 241 | P29464 | b0171 | Uridylate kinase (EC |
| | | | | | | | 2.7.4.—) |
| Rib | Y | Y | N | 217 | P24199 | b3041 | 3,4-dihydroxy-2- |
| | | | | | | | butanone 4-phosphate |
| | | | | | | | synthase |
| RibD | Y | Y | N | 367 | P25539 | b0414 | Riboflavin-specific |
| | | | | | | | deaminase/HTP |
| | | | | | | | reductase (EC |
| | | | | | | | 3.5.4.26/EC 1.1.1.193) |
| TdcG | Y | Y | N | 140 | P42630 | b3112 | L-serine dehydratase (EC |
| | | | | | | | 4.2.1.13) |
| ThiL | Y | Y | N | 325 | P77785 | b0417 | Thiamine- |
| | | | | | | | monophosphate kinase |
| | | | | | | | (EC 2.7.4.16) |
| YagS | Y | Y | N | 318 | P77324 | b0285 | Putative xanthine |
| | | | | | | | dehydrogenase yagS, |
| | | | | | | | FAD binding subunit |
| | | | | | | | (EC 1.1.1.204) |
| YahF | Y | Y | N | 515 | P77187 | b0320 | Predicted acyl-CoA |
| | | | | | | | synthetase subunit |
| YbeY | Y | Y | N | 155 | P77385 | b0659 | Predicted metal- |
| | | | | | | | dependent hydrolase |
| YbhA | Y | Y | N | 272 | P21829 | b0766 | Predicted HAD family |
| | | | | | | | hydrolase |
| YcdX | Y | Y | N | 245 | P75914 | b1034 | Predicted PHP family |
| | | | | | | | hydrolases |
| YciL | Y | Y | N | 291 | P37765 | b1269 | Predicted RluB-like |
| | | | | | | | pseudouridylate synthase |
| YdjQ | Y | Y | N | 295 | P76213 | b1741 | Predicted nuclease |
| YeaZ | Y | Y | N | 231 | P76256 | b1807 | Predicted protease |
| YfcH | Y | Y | N | 297 | P77775 | b2304 | Predicted nucleoside- |
| | | | | | | | diphosphate sugar |
| | | | | | | | epimerase |
| yfgE | Y | Y | N | 248 | P76570 | b2496 | DnaA paralog, predicted |
| | | | | | | | DNA replication |
| | | | | | | | initiation ATPase |
| YjeE | Y | Y | N | 153 | P31805 | b4168 | Predicted ATP/GTPase |

A genetic approach is used to validate the functionality of the recombinant enzymes. The rationale is to create a disruption in the chromosomal copy of the cognate gene in the presence of IPTG. The disruptions are made by the Wanner method (Datsenko et al. (2000) Proc. Natl. Acad. Sci. USA 97:6640-6645), which replaces an ORF with a kan resistance cassette. In parallel, disruptions are made in the wild type control. The disruption is verified by PCR amplification of the expected flanking region and verify its size. The ability to make a disruption in the ASKA strain expressing a recombinant enzyme, but not in the wild type control, indicates functional expression of the protein.

This set of 50 enzymes is smaller than the full set of strains carrying in vitro non-essential enzymes. Therefore, it is screened with a large, industry-size compound library to increase the probability of obtaining hits (e.g., a 500,000 compound library). Prodrugs are found among compounds that have antimicrobial activity. Therefore, the 500,000 compound library is first screened against wild type E. coli W3100, and the hits are reformatted into an active sublibrary.

In order to arrive at a realistic number of operations, the equivalent of 2 full library screens are performed (or 10⁶). The size of the active sublibrary is then 10⁶/50=20,000 compounds. A pilot screen is run with about 10,000 compounds of the original library to determine the hit rate at several concentrations (5, 10, 20 and 40 μg/ml), and then one can choose the one that produces an about 4% hit rate that will result in a 20,000 compound active sub-library. In order to differentiate between the control strain and one overexpressing an activating enzyme, the test compounds are applied at a concentration less than used to identify compounds active against the control E. coli. Therefore, the active sub-library of 20,000 compounds are retested against the wild type at 4 additional concentration, 10 μg/ml, 5 μg/ml, 2.5 μg/ml, and 1 μg/ml. In this manner, the approximate minimal active concentration for each compound is established. For testing prodrug candidates with strains overexpressing potential activating enzymes, compounds are tested at ⅕ minimal concentration obtained with the isogenic control strain. For this, compounds are grouped according to the concentration at which they are tested, in order to permit a uniform delivery of library compounds with pin dispensers.

Compounds that show activity against strains overexpressing the activating enzyme but not the wild type at the same test concentration are hits. The hits obtained are verified by retesting their activity against the overexpression and wild type strain. Verified hits are then validated by testing their activity against strains with diminished expression of the essential activating enzyme. Such strains are constructed using an antisense approach. High activity against the strain overexpressing the enzyme and low activity against a strain with diminished expression points to a candidate prodrug. Further validation includes measuring the MIC, MBC, sterilization activity against stationary cells, and ability to avoid TolC-dependent MDR efflux.

### Example 6

### Screening for Compounds Having Reduced Multidrug Pump Efflux

An ability of a prodrug to avoid MDR efflux is an additional indicator of the prodrug mode of action, and a predictor of a broad action spectrum. A test is used to ascertain this property of the prodrug hits. The rationale is to test the effect of a tolC mutation on drug susceptibility. TolC is an outer membrane porin used by the major E. coli transenvelope MDRs such as AcrAB for docking, and tolC mutants are very sensitive to antibiotics. A tolC::cam mutation is moved from an E. coli K12 tolC::cam into a strain deleted in the activating enzyme and the wild type, selecting for chloramphenicol resistance. The overexpression strain carries cam resistance of the plasmid. Therefore, in order to construct an overexpression strain deleted in tolC, a tolC::kan disruption cassette is moved from E. coli W3110 tolC::kan into it by P1 transduction. Comparable activity of each strain +/− the tolC mutation indicates the insensitivity to efflux. Note that having a number of compounds that can bypass MDR efflux in E. coli indicates that the screen is useful for discovering a broad-spectrum antiinfective.

### Example 7

### Inhibition of Multidrug Pump Efflux

Prodrugs are activated by activating enzymes into reactive molecules that bind to their targets, creating an irreversible sink. This may lead to insensitivity of the overall process to MDR efflux. In order to test this possibility, an E. coli K12 with a deletion in tolC coding for the outer membrane porin that is a component of the transenvelope MDRs was utilized (Li (2004) Drugs 64:159-204). This strain is highly sensitive to antibiotics (Tegos et al. (2002) Antimicrob. Agents Chemother. 46:3133-3141). Among the several E. coli MDRs that dock to TolC, AcrAB is significantly expressed and is primarily responsible for the intrinsic resistance of this bacterium to antibiotics. The substrate specificity of AcrAB is remarkably broad, and includes essentially all small molecular weight amphipathic compounds. The AcrAB substrates include anions (SDS, fatty acids, bile acids), neutral compounds (macrolides, chloramphenicol, tetracyclines) and cations or compounds that can form cations (acridine, quaternary compound antiseptics, fluoroquinolones). AcrAB can extrude a natural broad spectrum antibiotics that evolved for good penetration, chloramphenicol and tetracycline; and also extrude synthetic broad-spectrums, the fluoroquinolones. Compounds like fluoroquinolones or tetracycline are active against E. coli because they are used at levels that can overwhelm this and other MDRs. Knocking out tolC increases susceptibility at least 4 fold for the best penetrating compounds, and up to 1,000 fold for those that are good substrates of the pump (Tegos et al. (2002) Antimicrob. Agents Chemother. 46:3133-3141).

Cells were tested in a standard broth microdilution assay, inoculating 10⁵ cells per well of a microtiter plate in LB broth. Overexpression and deletion strains were compared to the appropriate isogenic wild type. IPTG was added at 1 mM to growth medium. Experiments involving nfnB and nfsA strains were performed under anaerobic conditions.

Amphipathic cations are useful substrates for all classes of MDRs, including the RND. See, e.g., Lewis (2001) J. Mol. Microbiol. Biotechnol. 3:247-254. Prodrugs metronidazole, dinitroaziridinylbenzamide and dinitrobenzamide, are amphipathic cations and are effectively extruded from E. coli.

There was no effect of tolC deletion on susceptibility of E. coli to prodrugs (Table 5). A control with erythromycin shows the typical decrease in MIC from 200 μg/ml in the wild type to 1.56 μg/ml in the ΔtolC strain, a 128 fold change. This observation suggests that prodrugs may counter efflux by activating into products that become trapped inside the cell by covalent attachment to their targets.

TABLE 5
The MIC (in μg/ml) of Prodrugs With E. coli Strains
Overexpressing and Deleted in the Activating Enzyme

| | Strain | | | |
|---|---|---|---|---|
| | | E. coli K12 | | |
| | | ΔtolC | E. coli K12 | |
| | | overexpressing | overexpressing | E. coli K12 |
| | E. coli | activating | activating | Δactivating |
| Compound | K12 wt | enzyme | enzyme | enzyme |
| Metronidazole | 563 | nfnB⁺ 8.8 | nfnB⁺ 8.8 | nfnB⁻ 1125 |
| | | nfsA⁺ 8.8 | nfsA⁺ 8.8 | nfsA⁻ 2250 |
| Dinitroaziridinylbenzamide | >200 | nfnB⁺ 3.3 | nfnB⁺ 3.3 | nfnB⁻ > 200 |
| (CB1954) | | nfsA⁺ 6.3 | nfsA⁺ 6.3 | nfsA⁻ > 200 |
| 3,5-Dinitrobenzamide | 250 | nfnB⁺ 15.6 | nfnB⁺ 15.6 | nfnB⁻ > 500 |
| | | nfsA⁺ 7.8 | nfsA⁺ 7.8 | nfsA⁻ > 500 |
| Fludarabine | >500 | deoD⁺ 125 | deoD⁺ 125 | deoD⁻ > 500 |
| 5-fluorocytosine | >2500 | codA⁺ 625 | codA⁺ 625 | codA⁻ > 2500 |
| Erythromycin | 200 | nfnB⁺ 1.56 | nfnB⁺ 200 | |
| | | nfsA⁺ 1.56 | nfsA⁺ 200 | |
| | | deoD⁺ 1.56 | deoD⁺ 200 | |
| | | codA⁺ 1.56 | codA⁺ 200 | |

### Example 8

### Screening For Sterilizing Prodrug Compounds

The bactericidal ability of the hits is then examined. This test probes the potential power of the screen to discover compounds capable of sterilizing an infection. While not a necessary property for an antibiotic, an ability to sterilize an infection is clearly advantageous, for example, in treating persistent biofilm infections and in biodefense applications.

The currently accepted measure of a killing ability of an antibiotic is MBC, the concentration of a drug capable of decreasing the level of cells in a logarithmically-growing population by ≧3 orders of magnitude. The experiment is performed as a usual MIC broth microdilution assay in microtiter plates, and the first, and two subsequent wells that show no visible growth are plated for colony counts to determine the MBC. The definition of MBC is useful in gauging the bactericidal ability of an antimicrobial compound. However, this test misses the persister cells present in all populations, and is inapplicable to stationary and biofilm cultures (Lewis (2001) Chemother. 45:999-1007; Coates et al. (2002) Nat. Rev. Drug Discov. 1:895-910). Most bactericidal antibiotics currently in use only act against rapidly growing cells. The FDA does not require testing developmental agents against stationary cultures. One of the results of this practice is the lack of compounds that are effective against biofilms, or other persistent infections.

For determining killing activity of hit compounds, the MBC is first measured by the standard broth microdilution method. Compounds that show considerable activity are examined in detail, with the aim of evaluating their ability to kill non-growing populations and persister cells. For this, dose-dependent killing experiments are performed with both log and stationary cultures of the wild type, and the strain overexpressing the cognate activating enzyme. Killing of non-growing cells is monitored by measuring the decline of viable cells of a stationary state population. The characteristic biphasic death observed with conventional antibiotics results from surviving persisters (Moyed et al. (1983) J. Bacteriol. 155:768-775; Spoering et al. (2001) J. Bacteriol. 183:6746-6751), and complete eradication of the culture indicates a sterilizing agent. Compounds that are able to kill non-growing cells and persisters in a wild type stationary population are of interest. Sterilization of an overexpression mutant, but not the wild type, also indicates a useful prodrug.

### Example 9

### Killing Ability of Metronidazole

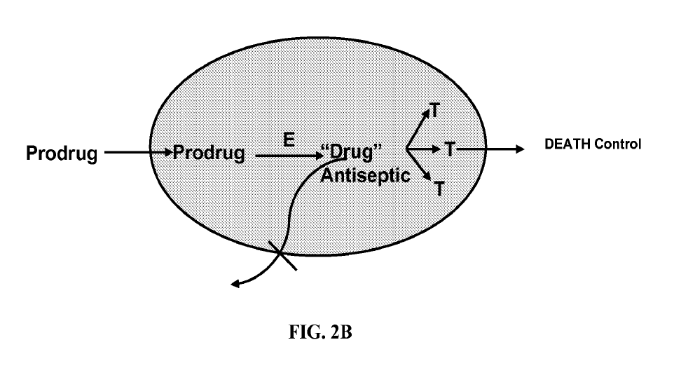

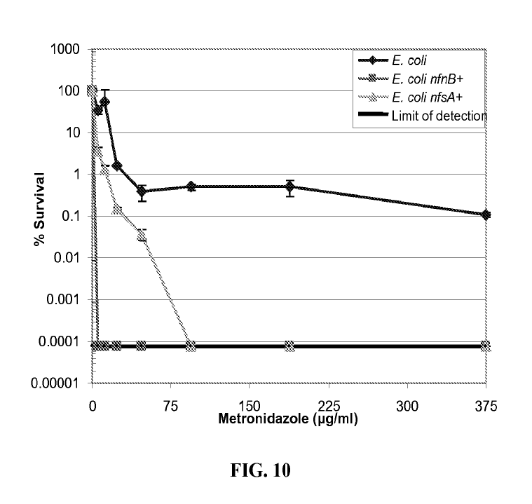

The killing ability of metronidazole was examined. The wild type bacterial strain was grown to stationary state under anaerobic conditions, and dose-dependent killing after incubation with metronidazole for 6 hours was detected by plating and colony count (FIG. 10). The wild type strain showed a typical biphasic killing, with about 1% of tolerant persister cells. Interestingly, no surviving persisters were detected in a strain overexpressing the activating enzyme, NfnB or NfsA. A complete sterilization of the population was observed with metronidazole tested against the nfnB⁺ strain (the line corresponding to 6 logs of killing is the limit of detection, <10 cells/ml). This is the first observation of a sterilizing activity of an antibiotic against a stationary state bacterial population. Previously, it was only possible to kill persisters and sterilize an infection with peracetic acid (FIG. 2); (Spoering et al. (2001) J. Bacteriol. 183:6746-6751). This experiment suggests that finding a prodrug with a better fit to its activating enzyme than metronidazole produces a useful therapeutic.

This result demonstrates the power of prodrugs to sterilize infections in immunocompromised patients, and to solve the intractable problem of multidrug tolerant infections such as biofilms. Candidate compounds that come out of the proposed screen can be developed into drugs that sterilize a broad range of pathogen infections.

### Example 10

### Persister Cells

In this example, multidrug tolerance of persister cells, which exemplifies the limitations of existing antibiotics, is characterized.

Persister cells in planktonic and biofilm populations are characterized for their antibiotic sensitivity in this example. Several bactericidal antimicrobials were chosen to test the resistance of P. aeruginosa to killing—ofloxacin, a fluoroquinolone; tobramycin, an aminoglycoside; carbenicillin, a β-lactam; and peracetic acid, a disinfectant oxidant (Spoering et al. (2001) J. Bacteriol. 183:6746-6751). Biofilms were grown essentially by the method of Ceri (Ceri et al. (1999) J. Clin. Microbiol. 37:1771-1776) and as described in Brooun et al. (2000) Antimicrob. Agents Chemother. 44:640-646. The device used for biofilm formation is a platform carrying 96 polystyrene pegs that fit in a microtiter plate. Preformed biofilms were incubated in the presence of an antimicrobial agent, and survival was measured after disrupting the biofilms by colony count.

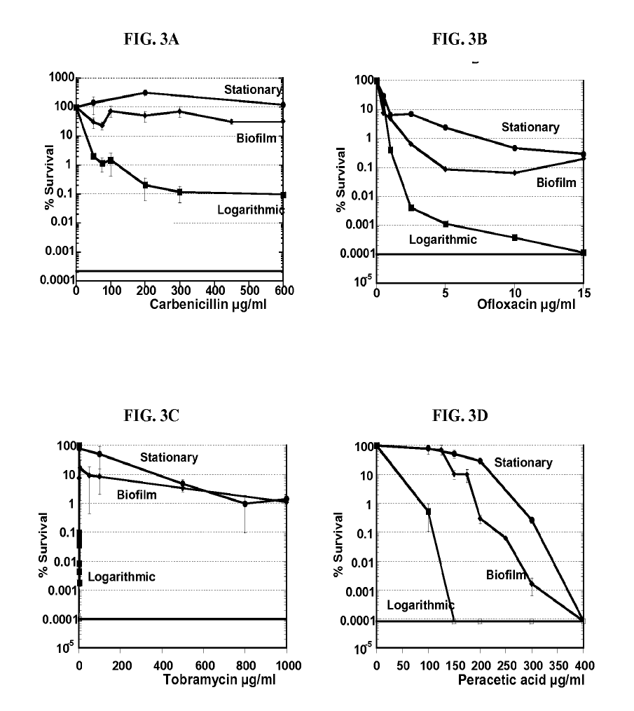

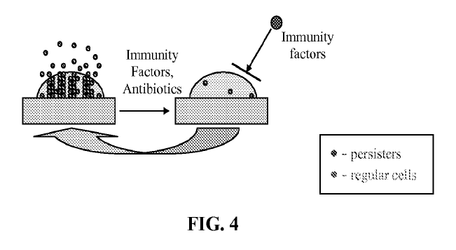

Logarithmic state, stationary and biofilm cultures were challenged with carbenicillin. Carbenicillin is a bactericidal antibiotic that, similarly to most β-lactams, only kills rapidly growing cells (Tuomanen et al. (1986) Scand. J. Gastroenterol. Suppl:10-14). Carbenicillin produced little killing in stationary cells, while the majority of logarithmic cells were killed at 1.67×MIC (FIG. 3A). The amount of killing of logarithmic cells approached a plateau at concentrations above 1.67×MIC, indicating the presence of a persister subpopulation. These 0.1% cells in the rapidly growing logarithmic culture were invulnerable to killing by carbenicillin at 600 μg/ml. Biofilm cells were resistant to killing by carbenicillin. This indicates that biofilms are made of slow growing cells.

Unlike carbenicillin, ofloxacin can kill non-growing cells (Brooun et al. (2000) Antimicrob. Agents Chemother. 44:640-646). Logarithmic, stationary and biofilm cultures were challenged with ofloxacin over a wide range of concentrations, from 1×MIC (0.5 μg/ml) to 30×MIC (15 μg/ml). After a 6 hrs incubation time with the antibiotic, viability was determined by colony count. The majority of cells in the three populations examined were killed at low concentrations of ofloxacin (FIG. 3B). The killing in all three cultures was distinctly biphasic, indicating the presence of persister cells. The levels of persisters were dramatically higher in the dense stationary planktonic and biofilm cultures as compared to logarithmic cells. The plateau at increasing concentrations of antibiotic shows that persisters are essentially invulnerable to killing by a fluoroquinolone. At 5 μg/ml ofloxacin, which is the clinically achievable concentration (Schulz et al. (1997) Pharmazie 52:895-911), the percentage of live cells was 0.001% in the logarithmic population, 0.1% in the biofilm and 2.5% in the stationary culture (FIG. 3B).

Logarithmic phase, stationary phase and biofilm cultures were challenged with tobramycin over a wide range of concentrations, from 1×MIC (1 μg/ml) to 1500×MIC (1500 μg/ml). Tobramycin was exceptionally effective in killing logarithmic cells and no logarithmic persisters were detected (FIG. 3C). Tobramycin at 50 μg/ml (the maximal clinically achievable concentration is 10 μg/ml), (Schulz et al. (1997) Pharmazie 52:895-911) eliminated 90% of the biofilm cells, and the remaining population declined gradually with increasing amounts of the antibiotic. At high concentrations surviving persisters became apparent. Tobramycin was ineffective against stationary planktonic cells, apparently due to the dependence of killing of growth.

Peracetic acid, an oxidizing disinfectant, was used in order to test the ability of a sterilizing agent to act against persisters. Biphasic killing was not observed for this antimicrobial agent (FIG. 3D), and all cultures were sterilized. Antibiotics acting against specific targets are inactive against persisters, and their elimination requires a general disinfectant/antiseptic compound. Similar results showing biphasic killing, and high level of persisters in stationary and biofilm populations, also were obtained with S. aureus and E. coli (Keren et al. (2004) FEMS Microbiol. Lett. 230:13-18).

The data described above provide an insight into the recalcitrant nature of biofilm infections. Antibiotics like ofloxacin eliminate most of the cells of an infecting biofilm but leave persisters intact (FIG. 3). The immune system is likely to eliminate the remaining planktonic persisters (similarly to eliminating live planktonic cells after treatment with a static antibiotic). However, biofilm cells are physically protected from the components of the immune system by the exopolymer matrix. Eradication of planktonic cells eliminates the symptoms of disease, and antibiotic treatment is discontinued. Once the antibiotic level drops, persisters reform the biofilm, which sheds off new planktonic cells. This model explains the relapsing nature of biofilm infections, and the need for a lengthy antibiotic therapy. Other persistent infections, for example, non-biofilm infectious diseases in immunocompromised individuals, follow a similar pattern of population regrowth stemming from surviving persister cells.

### Example 11

### Multidrug Tolerance Genes in E. coli

Persisters are apparently dormant cells, and this was tested directly by examining their capability for protein synthesis.

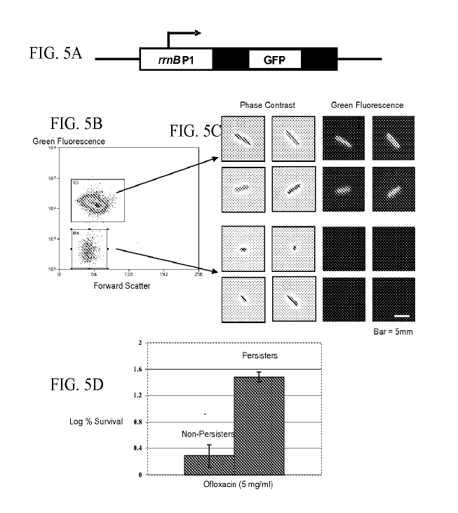

In E. coli ASV, a degradable GFP is inserted into the chromosome in the λ, attachment site and expressed from the ribosomal rrnBP1 promoter, the activity of which is proportional to the rate of cell growth (FIG. 5A). The half-life of degradable GFP is greater than 1 hr, and it should is cleared from dormant cells. This enables sorting of dim persister cells.

A logarithmically-growing population of E. coli ASV was sorted with a MoFlo cell-sorter using forward light scatter, which allows detection of particles based on size. This enabled detection of cells irrespective of their level of fluorescence. Fluorescence of GFP in individual cells was recorded simultaneously using laser excitation and light detection. FACS analysis showed that the population consisted of two strikingly different types of cells, (a bright majority, and a small subpopulation of cells with no detectable fluorescence (FIG. 5B). The two populations were sorted based on fluorescent intensity and collected in phosphate buffer. Epifluorescent microscopy confirmed that the sorted bright cells were indeed bright green, while the dim ones had no detectable fluorescence (FIG. 5C). The dim cells were also smaller than the fluorescent cells, and in this regard resembled stationary state cells. Sorted dim cells were exposed to a high level of ofloxacin that rapidly kills both growing and non-growing normal cells, but has no effect on persisters (Spoering et al. (2001) J. Bacteriol. 183:6746-6751; Keren et al. (2004) FEMS Microbiol. Lett. 230:13-18). The majority of this subpopulation survived, as compared to a drastic drop in viability of the sorted bright cells (FIG. 5D). This experiment shows that the sorted dim cells are dormant persisters.

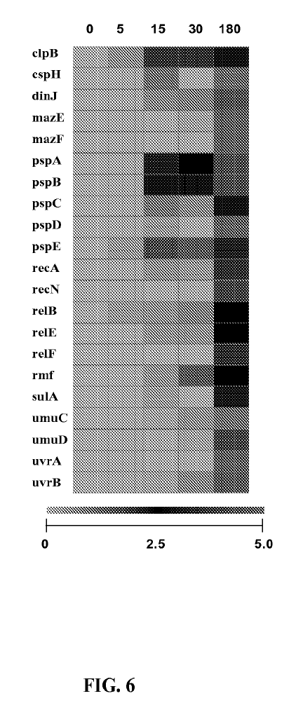

To identify candidate persister genes, an expression profile from persister cells was identified. This was done both from sorted cells (in analysis) and from persisters collected after lysis of a growing population with ampicillin (Keren et al. (2004) J. Bacteriol. 186:8172-8180). Genes expressed in persisters that could create a dormant state were sought. The profile indicated several candidates: RMF inhibits translation by forming ribosome dimers (Wada, A. (1998) Genes Cells 3:203-208); UmuDC has been reported to inhibit replication (Opperman et al. (1999) Proc. Natl. Acad. Sci. USA 96:9218-9223); and SulA is an inhibitor of septation (Walker (1996) Cell Mol. Biol. Neidhardt, F. C. (ed). Washington, D.C.:ASM Press, pp. 1400-1416) (FIG. 6).

E. coli HM22 hipA7 cells were grown in LB medium to mid-exponential phase (about 5×10⁷ cells/rill) at 37° C. with aeration and treated with 50 μg/ml ampicillin. After the culture lysed, remaining persisters were sedimented and the isolated RNA was used for microarray analysis. The heatmap of expressed genes was generated with Spotfire Decisionsite 7.2.

There was overexpression of well-characterized chromosomal toxin-antitoxin (TA) modules RelBE, MazEF, and DinJ/YafQ, a homolog of RelBE. Homologs of these genes are found on plasmids where they constitute a maintenance mechanism (Hayes (2003) Science 301:1496-1499). The ability of “toxin” modules to reversibly block translation makes them excellent candidates for multidrug tolerant (MDT) genes. By shutting down antibiotic targets, toxins can produce multidrug tolerant cells.

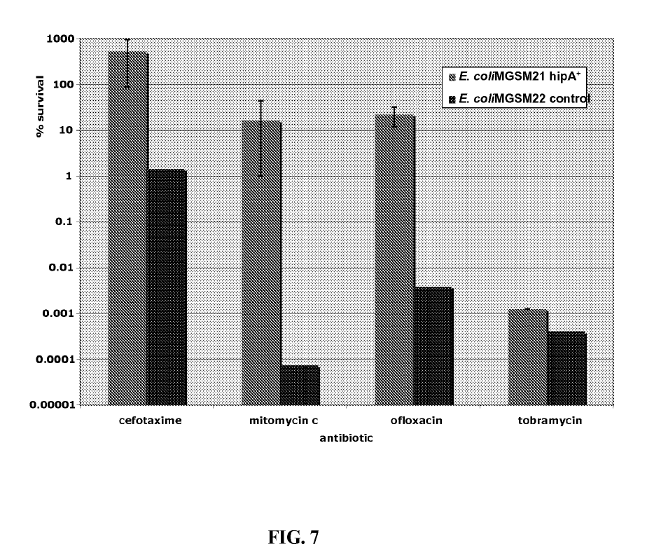

Overexpression of recombinant RelE increased the level of persisters surviving treatment with cefotaxime, ofloxacin and tobramycin 10-10,000 fold (not shown). Expression of another toxin, HipA, strongly protected cells from killing by antibiotics as well (FIG. 7).

### Example 12

### Multidrug Tolerance of E. coli Expressing HipA

Strains MGSM21(pBAD33::hipA) and MGSM22(pBAD33) were grown to OD₆₀₀=0.3, at which point L-arabinose was added to induce expression of HipA from pBAD33. After 30 min a 1.0 ml aliquot of each strain was challenged with either cefotaxime (100 μg/ml), Mitomycin c (10 μg/ml), ofloxacin (5 μg/ml), or tobramycin (25 μg/ml) for 3 hrs, at 37° C. with aeration. Cells were collected, washed once, diluted, and spot plated to determine CFU's. After treatment with antibiotic, cells were plated on media without inducer and were allowed to recover.

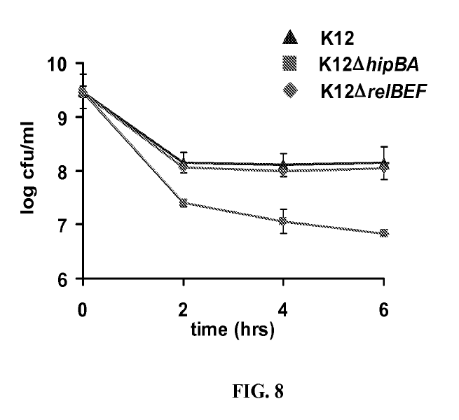

Strains deleted in relBE; mazEF; or hipBA were created (Datsenko et al. (2000) Proc. Natl. Acad. Sci. USA 97:6640-6645) and tested for persister production in both growing and stationary cultures. Antibiotics exhibiting lethal action in stationary cultures are essentially limited to the fluoroquinolones and mitomycin C. Both antibiotics produced a sharp (10-100 fold) decrease in stationary cell persisters in the ΔhipBA strain (FIG. 8) and in a biofilm.

Deletion of hipBA had no effect on the MIC of antibiotics. When tested in a logarithmic culture, or in stationary state minimal medium, cells deleted in the hipBA locus did not show a lower level of persisters as compared to the isogenic parent strain. Other MDT genes play a leading role in persister formation under those conditions. Deletion of either relBE (FIG. 8), mazEF, dinJ/yafQ, or rmf did not affect persister production. TA modules are highly redundant, and creating a multiply deleted strain will probably reveal the identity of additional MDT genes that play a role in logarithmic state cells.

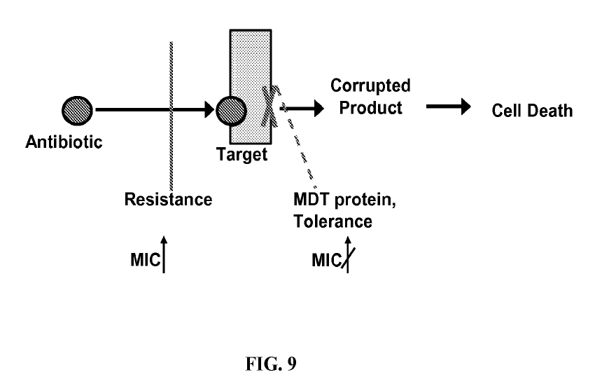

Based on these findings, the following model of persister production and antibiotic tolerance is presented (FIG. 9). The ratio of a toxin/antitoxin (such as HipA/HipB) in a population fluctuates, and rare cells express relatively high levels of a toxin. Bactericidal antibiotics bind to a target protein and corrupt its function, generating a lethal product (for example, aminoglycosides interrupt translation, resulting in misfolded peptides that damage the cell). A toxin binds to the target and inhibits the function, leading to tolerance. The antibiotic can bind to the blocked target, but can no longer corrupt its function. Inhibition of translation by a toxin further causes a relative increase in the stable toxin (due to antitoxin degradation) of this and other TA modules, which might has an autocatalytic effect on inhibition of translation, leading to a shutdown of other cellular functions, and to dormant, tolerant persister cells.

### Example 13

### Animal Studies to Determine Pathogen Prodrug Activating Gene Function in a Host

The activating enzymes identified in the above screens are then examined for in vivo essentiality in a host. E. coli has a number of enzymes well conserved among bacteria, and some of them are essential in the challenging environment of the host. An example of 30 well-conserved enzymes that do not have close human homologs, but are non-essential in vitro is given below (Table 6).

The in vivo essentiality of the activating enzymes that are identified is tested following the rate of clearance of E. coli O157:H7 in a mouse model of gastrointestinal infection. Disruptions of these genes in E. coli 0157:H are made by the Wanner procedure (Keren et al. (2004) FEMS Microbiol. Lett. 230:13-18).

TABLE 6
Conserved Non-Essential E. coli Enzymes

| E. coli | Mtub | Essential | Human | Annotation |
|---|---|---|---|---|
| Alr | Rv3423c | No | No | Alanine racemase |
| AroA | Rv3227 | No | No | 5-enolpyruvylshikimate-3-phosphate |
| | | | | synthase |
| AroB | Rv2538c | No | No | 3-dehydroquinate synthase |
| AroE | Rv2552c | No | No | Shikimate dehydrogenase |
| HisB | Rv1601 | No | No | Imidazoleglycerol-phosphate dehydratase |
| HisD | Rv1599 | No | No | Histidinol dehydrogenase |
| HisF | Rv1605 | No | No | Imidazoleglycerol-phosphate synthase |
| HisG | Rv2121c | No | No | ATP phosphoribosyltransferase |
| HisH | Rv1602 | No | No | Glutamine amidotransferase |
| MazG | Rv1021 | No | No | Predicted NTP pyrophosphatase |
| Mqo | Rv2852c | No | No | Malate:quinone oxidoreductase |
| PaaD | Rv1466 | No | No | Predicted component of oxygenase/ring |
| | | | | hydroxylase |
| Pta | Rv0408 | No | No | Phosphotransacetylase |
| TldD | Rv2315c | No | No | Microcin B17 maturation protease |
| WbbL | Rv3265c | No | No | Rhamnosyl transferase |
| YaeI | Rv3683 | No | No | Predicted phosphodiesterase |
| YcdH | Rv3567c | No | No | Flavin reductase |
| YedY | Rv0218 | No | No | Sulfite oxidase-relate molybdoenzyme |
| YfgB | Rv2880c | No | No | Pyruvate formate lyase activating enzyme |
| YgfJ | Rv0371c | No | No | MobA paralog, predicted molybdopterin- |
| | | | | guanine dinucleotide biosynthesis enzyme |
| YgiN | Rv2749 | No | No | Predicted monooxygenase |
| YhbJ | Rv1421 | No | No | Predicted P-loop-containing kinase |
| YhgH | Rv3242c | No | No | Predicted amidophosphoribosyltransferase |
| YidA | Rv3813c | No | No | Predicted phosphatase |
| YjbQ | Rv2556c | No | No | Predicted His, Asp-dependent enzyme |
| YjeQ | Rv3228 | No | No | Predicted GTPase |
| YjfR | Rv0906 | No | No | Predicted metallohydrolase (beta-lactamase |
| | | | | superfamily) |
| YjgR | Rv2510c | No | No | Predicted ATPase |
| YpfJ | Rv2575 | No | No | Predicted metalloprotease |
| YraL | Rv1003 | No | No | Predicted methyltransferase |
| ElaA | Rv2851c | Y/N | No | Predicted acyltransferase |
| YidD | Rv3922c | Y/N | No | Predicted Cys-dependent enzymes |
| HisI | Rv1606 | No | No | Phosphoribosyl-AMP cyclohydrolase |
| AbgA | Rv3305c | No | Y/N | Metal-dependent amidase/aminoacylase |
| AbgB | Rv3306c | No | Y/N | Metal-dependent amidase/aminoacylase |
| AroL | Rv2539c | No | Y/N | Shikimate kinase |
| CaiE | Rv3525c | No | Y/N | Carbonic anhydrase/acetyltransferase |
| MazG | Rv1021 | No | Y/N | NTP pyrophosphatase |
| MhpC | Rv3569c | No | Y/N | Predicted hydrolase/acyltransferase |
| YbaW | Rv2475c | No | Y/N | Predicted thioesterase |
| YcdJ | Rv0554 | No | Y/N | Predicted hydrolase/acyltransferase |

6 to 8 week old female ICR (CD-1) mice are used for in vivo studies. The animals are infected with the wild type, and the course of the infection is followed. As a control for essentiality, a strain with a chromosomal knockout of an essential dihydrodipicolinate reductase (dapB), expressing the enzyme from a regulated promoter on a recombinant vector is used. The plasmid is moved by transformation from E. coli K12 of the ASKA library into E. coli O157:H7. A knockout of the chromosomal copy is then made as described above. This strain is dependent on IPTG for growth, and is expected to be unviable in vivo. Leakage from the promoter may be sufficient for this strain to grow in the absence of added inducer. This strain is not expected to cause disease, and should rapidly clear from the animals. Clearance is followed by plating samples on kanamycin medium (kan resistance is carried by the disruption cassette). In order to follow clearance of the test strains lacking activating enzymes, these are similarly plated on kanamycin medium. The rate of clearance of this strain serves as a benchmark to evaluate possible in vivo essentiality of the test strains. Those that fail to cause disease and exhibit clearance comparable to the dapB control will signify in vivo essentiality of the cognate gene. The hits that are converted by these in vivo essential enzymes are then entered into the drug development process. The strains overexpressing these in vivo essential enzymes are used together with strains overexpressing in vitro essential enzymes in a different modality of the screen.

Enterohemorragic E. coli O157:H7 strain EDL 933 (ATCC 700927), which produces both Shiga-like cytotoxins (SLT-I and SLT-II), are used in the study and serve as a positive control. In order to test in vivo essentiality, a set of 30 strains deleted in conserved non-essential enzymes is prepared (Table 7) as described above. A strain carrying a disruption in an essential gene dapB and expressing it from a plasmid in response to IPTG serves as a negative control. Knockout strains (CAT^{R}) is gown in LB broth at 37 C for 16 to 18 hr, diluted 1/1000 in fresh LB broth, cultured to mid-log phase, harvested by centrifugation, washed twice in phosphate-buffered saline (PBS, pH 7.4) and resuspended in PBS. Chloramphenicol will be added to media at a final concentration of 25 μg/ml.

Chromosomal genes are disrupted using linear DNA fragments with short (about 50 bp) terminal homologies to the targeted gene(s) and phage λ Red recombinase. A gene cassette encoding kanamycin resistance is amplified by PCR using primers with 5′-extensions that are homologous to regions adjacent to the targeted gene, and the PCR fragment is electroporated into cells expressing Red recombinase encoded by a helper plasmid. Kanamycin resistant colonies with the resistance cassette integrated into chromosome are isolated and verified by PCR. The temperature sensitive helper plasmid is then cured.

Animals are allotted one per cage and allowed to acclimate (free access to water and food) for at least three days upon arrival at the experimental facility. Animals are then starved for water and food for 18 hr, infected the next morning by intragastric gavage with 10⁸ cells of the desired E. coli O157:H7 strain and then allowed access to food and water ad libitum. Each strain is tested in triplicate. The control strain expressing an essential IPTG-inducible DapB is expected to rapidly disappear from the animals. As a negative control, a group of animals receives only sterile PBS, while the positive control animals receive 10⁸ cells of the wild type E. coli O157:H7 EDL933 strain. For the following 15 days, animals are controlled daily for feed and water intake, weight, and general health status. Feces are collected for E. coli O157:H7 counts, dry-matter measurements, and fecal occult blood detection.

At days 5, 10 and 15 three animals per each group are euthanized via CO₂ inhalation and samples of: mucosa from proximal, middle and distal small intestine, cecum, and proximal, and distal colon, feces, urine and blood are collected for E. coli O157:H7 counts. At the same time, each mouse undergoes a full-necroscopy examination and specimens of: proximal, middle and distal small intestine, cecum, colon, liver, spleen and kidneys are collected, fixed in 10% buffered neutral formalin, and processed for further histological examinations.

The in vivo experiments point out possible in vivo essentiality of “in vitro non essential genes,” by tracking clearance (survival) of knockout strains of E. coli O157:H7 EDL933. At the same time the sampling and analysis procedures allows a determination of the role of tested genes in: time-course of infection establishment, severity of disease, ability of E. coli O157:H7 EDL933 to colonize intestinal mucosa and degree of intestinal lesions, fecal shedding of the pathogen, as well as systemic lesion in more sensitive organs (kidneys, liver and spleen) and possible septicemia occurrence. Overtime observations of animals' behavior relative to feed and water intake, general health status and detection of occult blood in feces reliably allow the recognitions of the occurrence of a sub-clinical status of infection.

### Example 14

### Prodrug Antibiotic Screens Using Essential Genes

A. Strains Diminished in Expression of Essential Enzymes

In order to focus specifically on the essential activating enzymes, a set of strains with diminished expression is constructed. As mentioned above, decreased expression of an essential target leads to an increase in activity of a conventional antibiotic, and this property has been used by the industry for whole-cell screening for compounds hitting this target. Diminished expression leads to decreased activity of a prodrug, and this serves to specifically identify these compounds, similarly to screening a knockout library described above.

There are about 300 essential genes in E. coli, 250 of which are suitable enzymes. An antisense RNA approach is used to construct a set of E. coli strains with diminished expression of 250 essential enzymes. A large-scale construction of antisense strains has been successfully employed before to identify essential enzymes in S. aureus (Ji et al. (2001) Science 293:2266-2269). A similar strategy is utilized, and follows the specific protocols for antisense suppression of target genes developed for E. coli (Chen et al. (2003) Antimicrob. Agents Chemother. 47:3485-3493; Stefan et al. (2003) FEBS Lett. 546:295-299; Wang et al. (2003) FEMS Microbiol. Lett. 220:171-176). DNA fragments coding for a given enzyme are amplified by PCR. Primers carrying restriction sites are used, enabling cloning of the amplified DNA into pCA24N vector in the antisense orientation relative to the pT5lac promoter. Resulting constructs enable controlled expression of antisense RNA through induction with IPTG (see Methods for details). Strain construction is streamlined and cloning procedures performed in parallel in microtiter plates.

Addition of IPTG at a saturating concentration (3 μM) leads to a high level of antisense RNA expression. All recombinant strains are therefore cultured in plates with 3 μM IPTG, and lack of growth signifies successful construction. Next, the level of IPTG is determined for each strain that decreases growth rate. This is performed in 384 well microtiter plates under conditions similar to screening. Each strain is inoculated into a well with one of 5 IPTG concentrations and a control. This results in a total of 1250 wells, or 4 plates. The experiment establishes the level of expression that still gives good growth (about 20% inhibition), suitable for screening, but a decreased level of an activating enzyme sufficient to provide increased resistance to a prodrug. This screen selects prodrug hits that have decreased activity in this screen as compared to the wild type.

One of the 5 tested concentrations of IPTG results in a suitable level of essential enzyme expression for most if not all of the strains. The 250 strains expressing antisense RNA are grouped into 5 distinct sets, and each is screened separately. This means 5 separate screens of a 150,000 compound library. Prodrugs are found among compounds that have direct antimicrobial activity. Therefore, the 150,000 compound NERCE library is screened against wild type E. coli W3110. The hit rate for antimicrobials in a compound library against E. coli is ≦0.5%. Taking the higher estimate of 5%, this will result in 7,500 compounds with direct activity. These are then picked by a “cherry-picking” robot and reformatted into a sublibrary. Screening this sublibrary against 5 pools of strains and a wild type control is equivalent to a screen of 45,000 compounds.

Subsequent steps of verifying and validating the hits are similar to those described above. Briefly, the hits that allow growth in a pool of strains with diminished enzyme expression signify possible prodrug compounds. A secondary screen involves testing the hit compound against individual strains of the mix which identifies the strain resistant to a particular compound. Next, validation is performed with a strain from the ASKA library overexpressing the enzyme. An increased susceptibility against a strain overproducing an enzyme as compared to a strain with diminished expression serves as an initial validation for a prodrug. The wild type is incorporated in this test as well. This experiment is performed with a range of hit concentrations and produces an MIC for the strains, including the wild type. Hits with the lowest wild type MIC are then focused on. The hits are then examined for their ability to avoid MDR efflux by testing their activity in a tolC background, and for their ability to sterilize a stationary state population, as described above.

B. Creating Strains with Controlled Gene Expression Using an Antisense Approach

DNA fragments coding for the chosen enzymes are amplified by PCR using corresponding clones from the ASKA overexpression library as template and a pair of primers complementary to the vector sequence flanking the cloned ORFs. Primers also carry restriction sites enabling cloning the amplified enzyme coding inserts into pCA24N vector (GeneBank accession number AB052891) in the antisense orientation relative to the pT5lac promoter in the vector. Resulting constructs enable controlled expression of antisense RNA through the induction with IPTG. Tight repression before induction is ensured by the expression of the lac^{q} gene cis-encoded on the same vector (Chen et al. (2003) Antimicrob. Agents Chemother. 47:3485-3493; Stefan et al. (2003) FEBS Lett. 546:295-299; Wang et al. (2003) FEMS Microbiol. Lett. 220:171-176).

### Example 15

### Screening for Additional Compounds

A. Screen #1

An additional screen of 42,822 compounds was performed according to the methods described in Example 1. Two screened molecules scored as prodrug hits and 96 displayed direct inhibitory activity against the wild type. Among the prodrug hits, several compounds were noted that are known prodrugs or are related chemically to known prodrugs, including zidovudine (AZT, the AIDS therapeutic, which is a known prodrug for E. coli (Lavie et al. (2004) Mini Rev. Med. Chem. 4:351-359)) and thiourea, N,N′-bis(3-chlorophenyl)-(9CI), referred to as “PD18”:

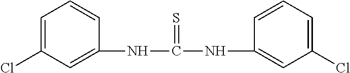

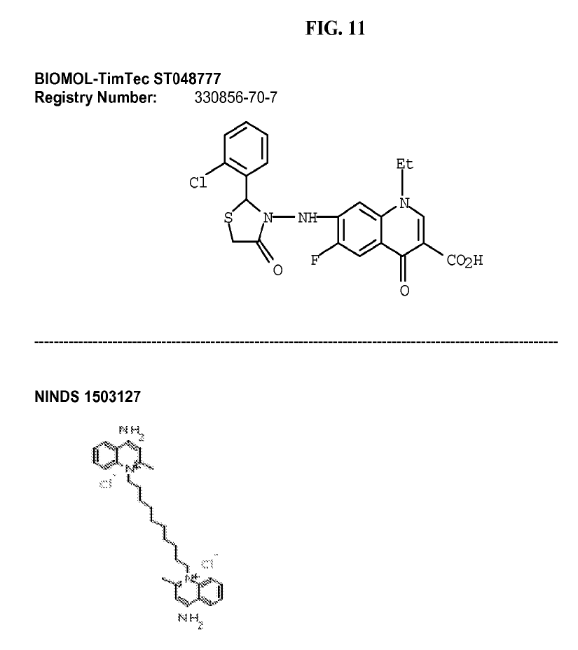

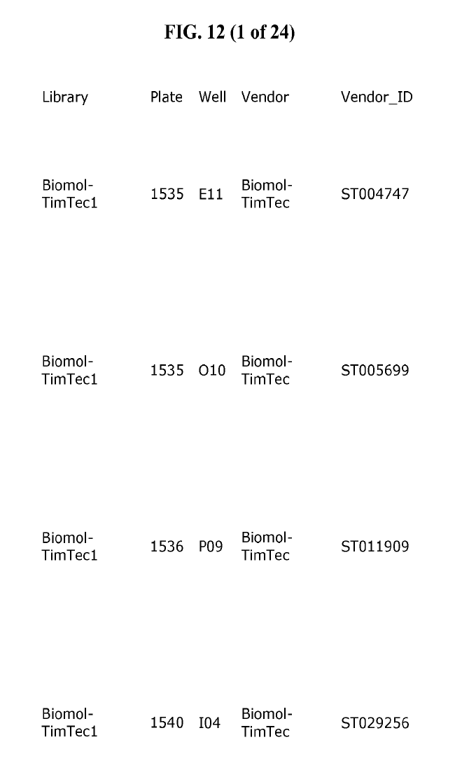

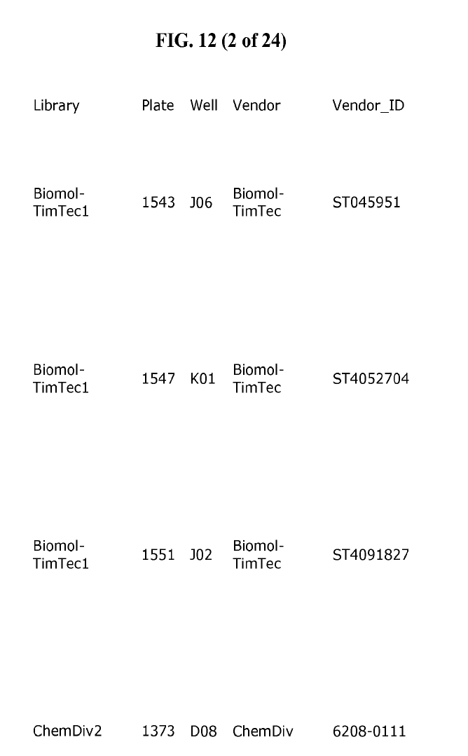

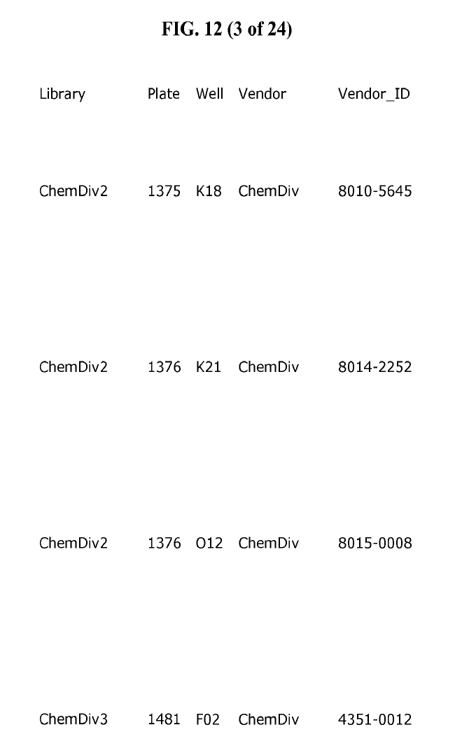

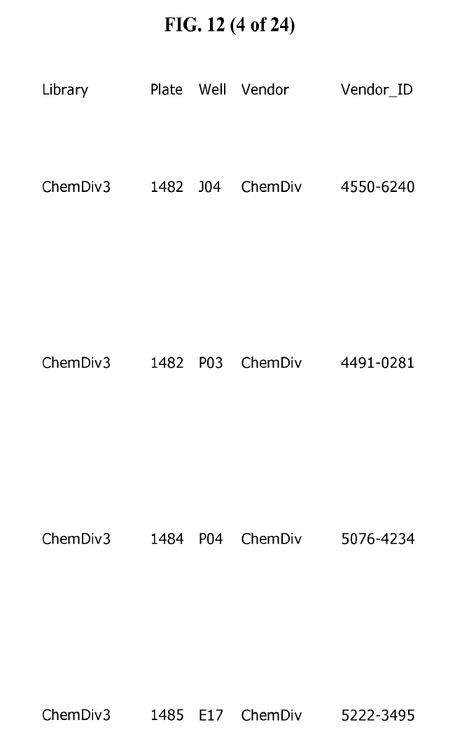

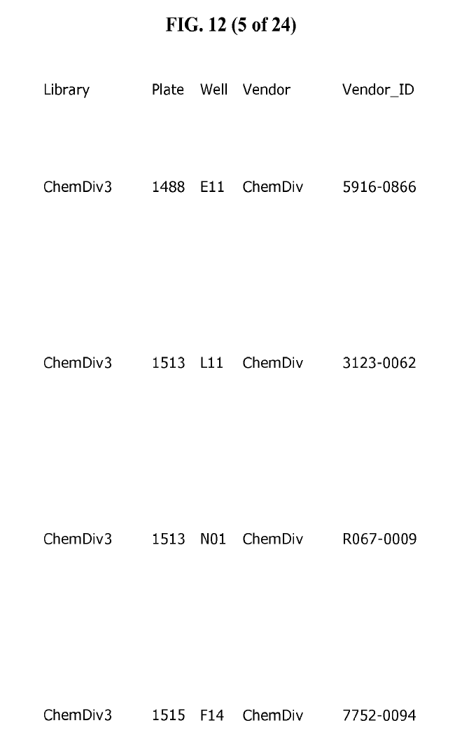

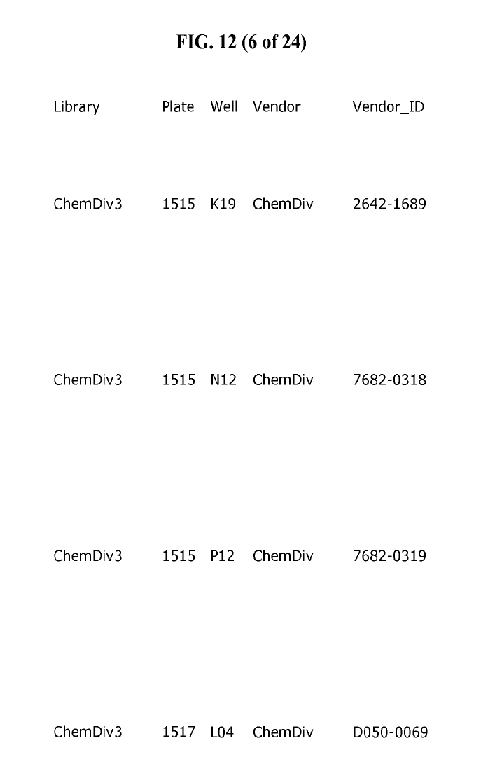

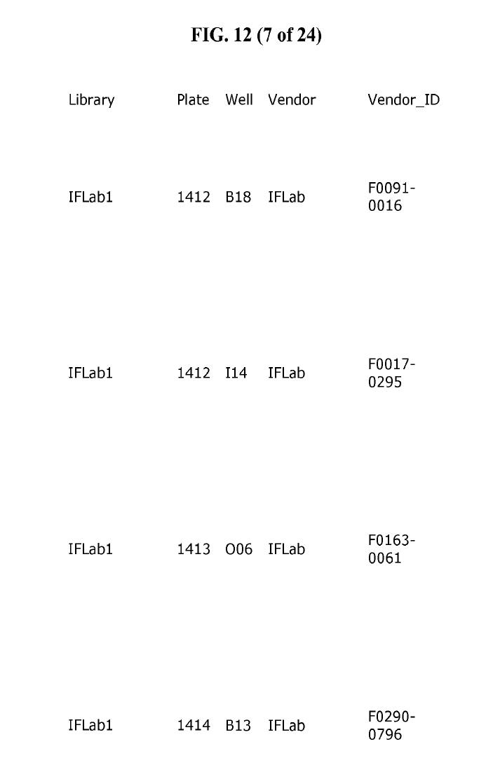

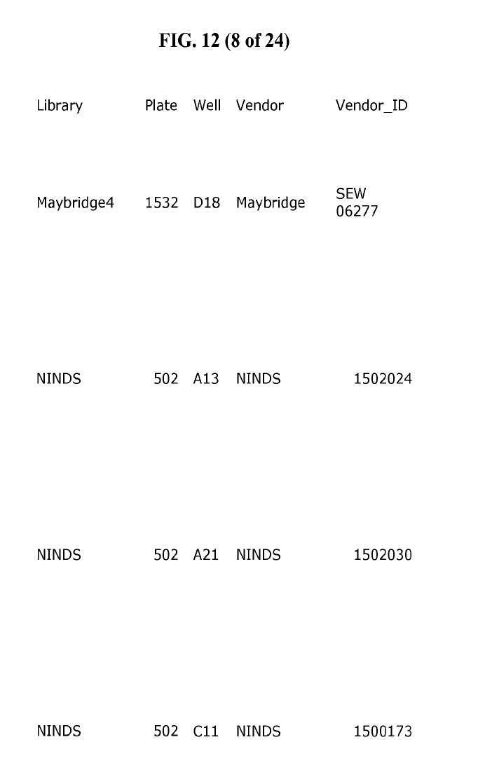

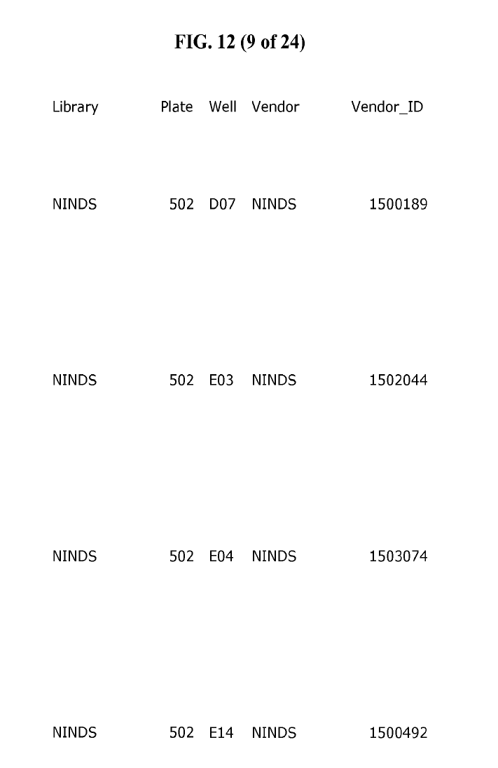

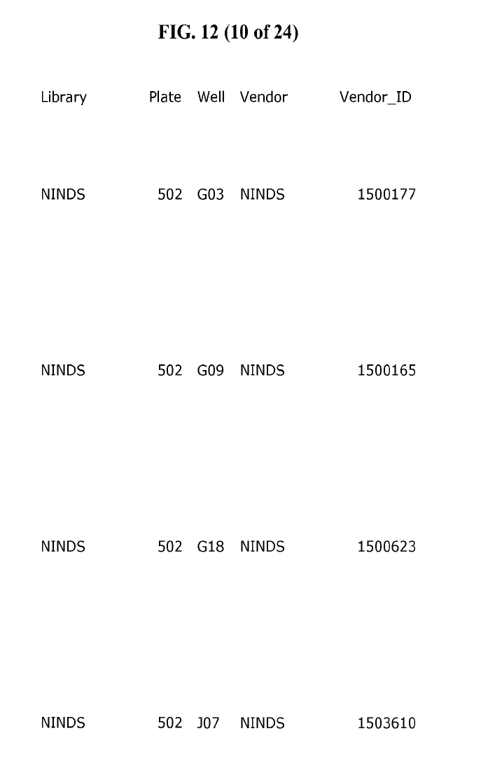

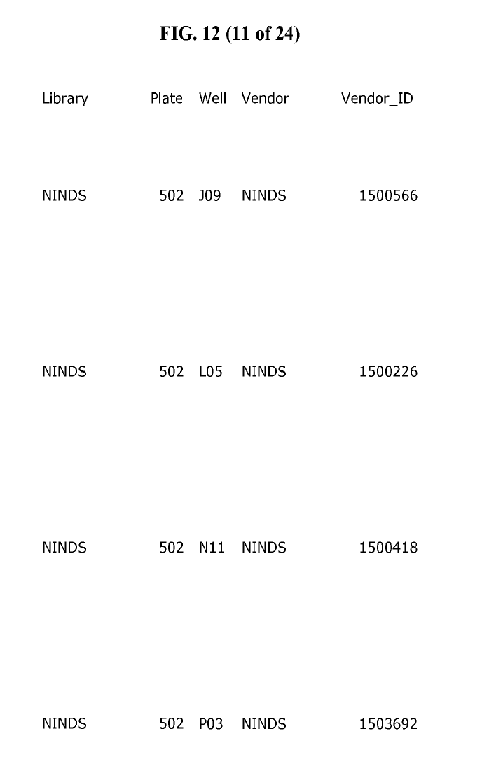

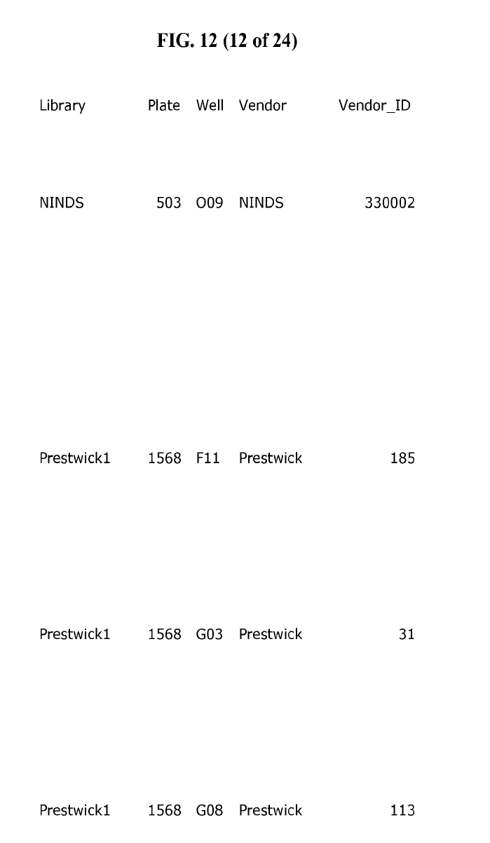

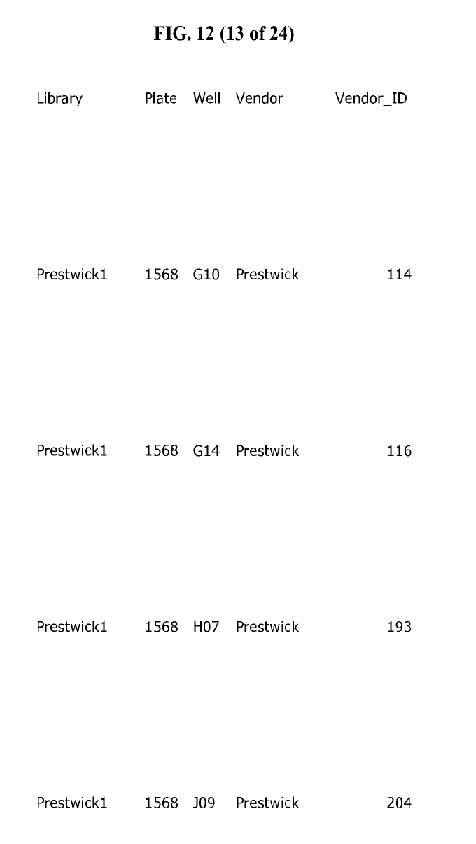

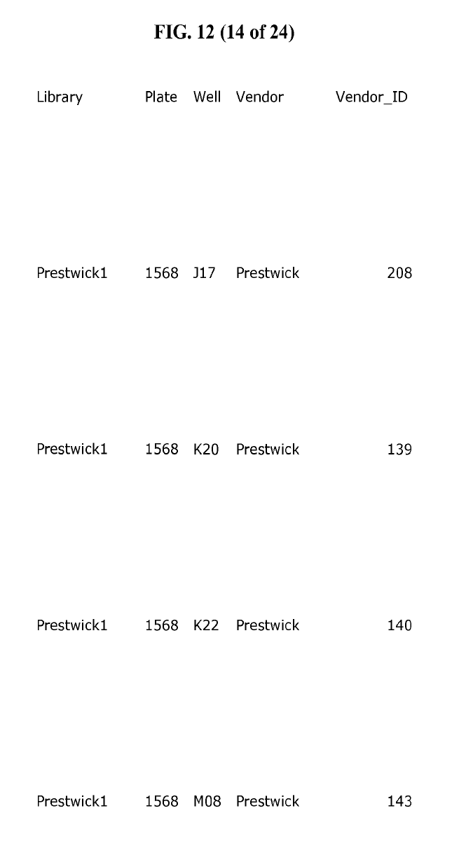

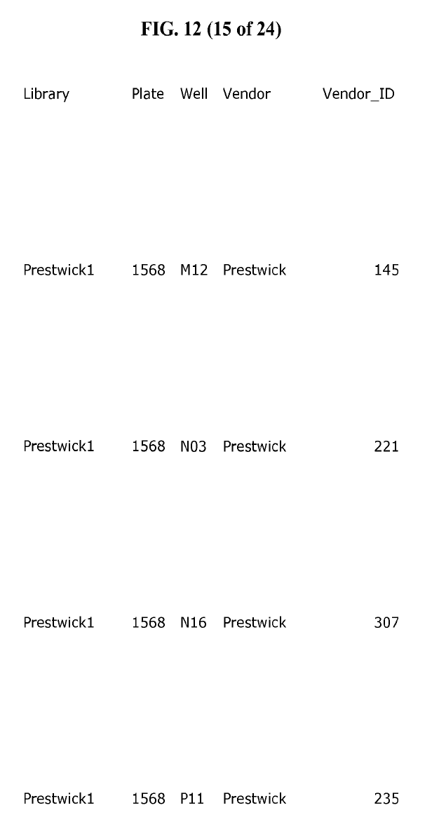

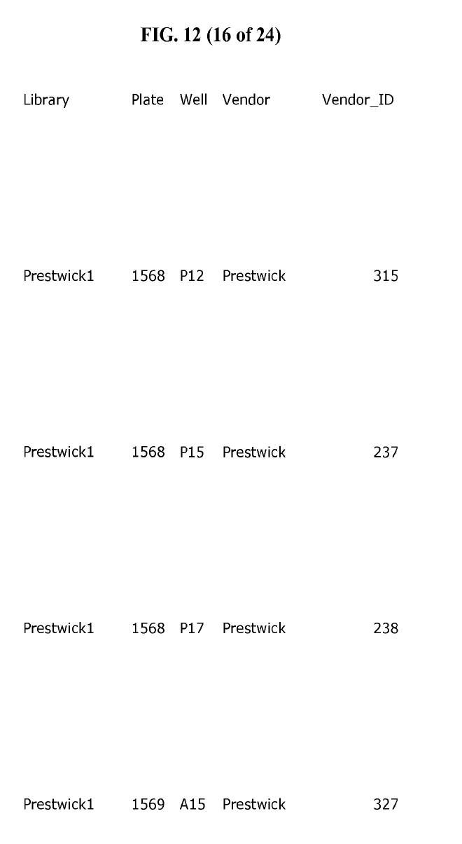

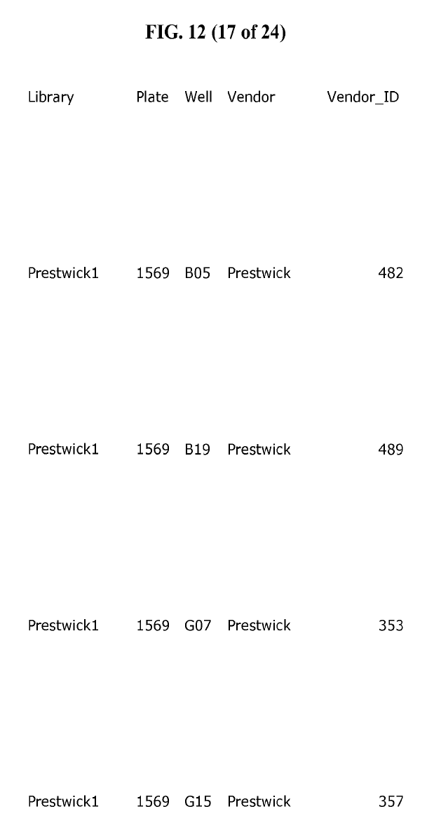

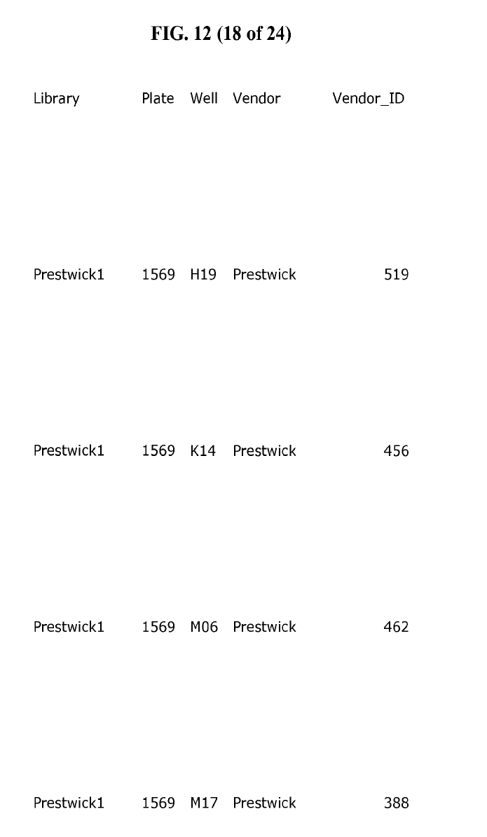

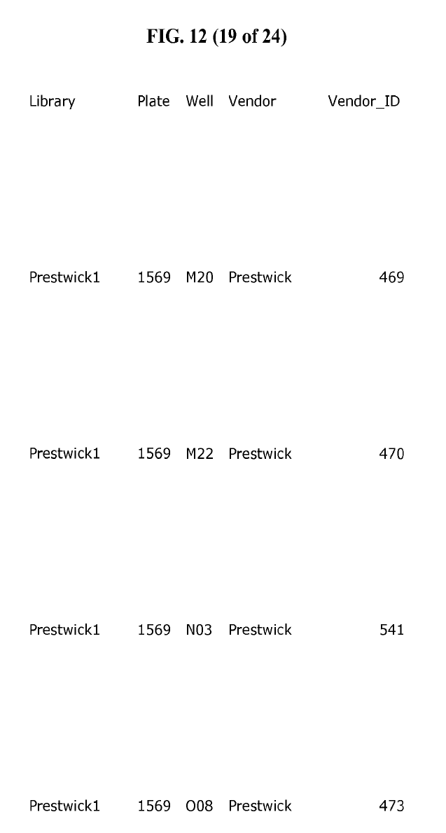

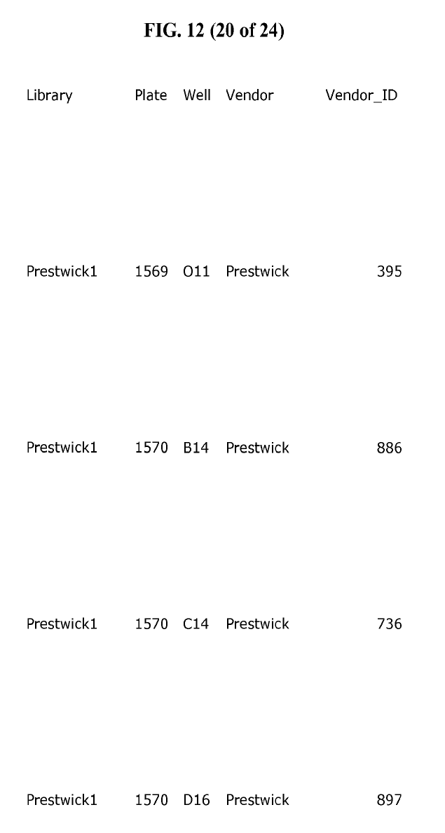

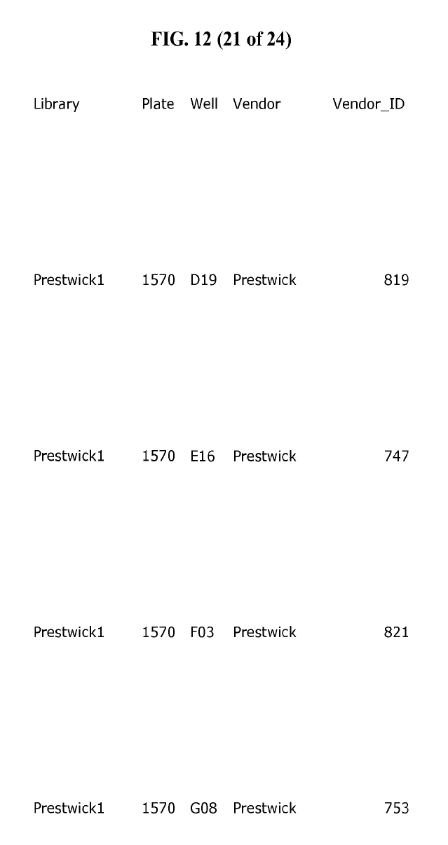

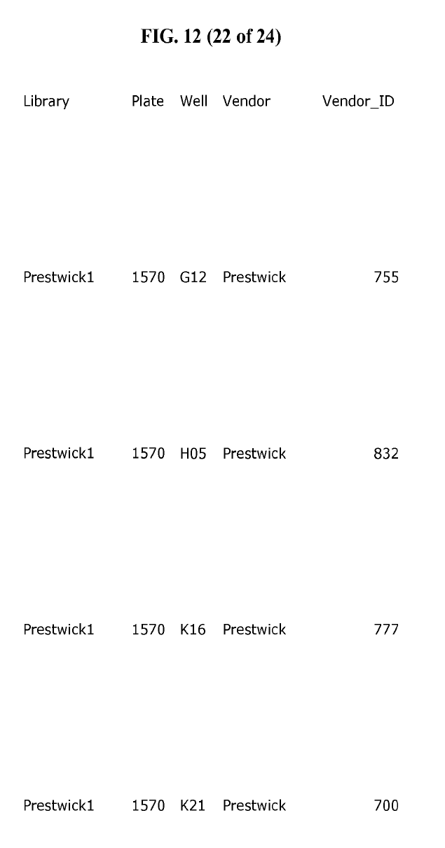

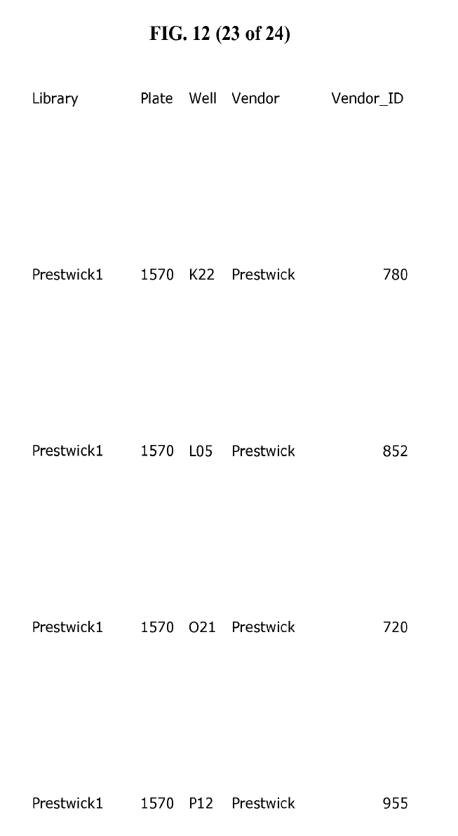

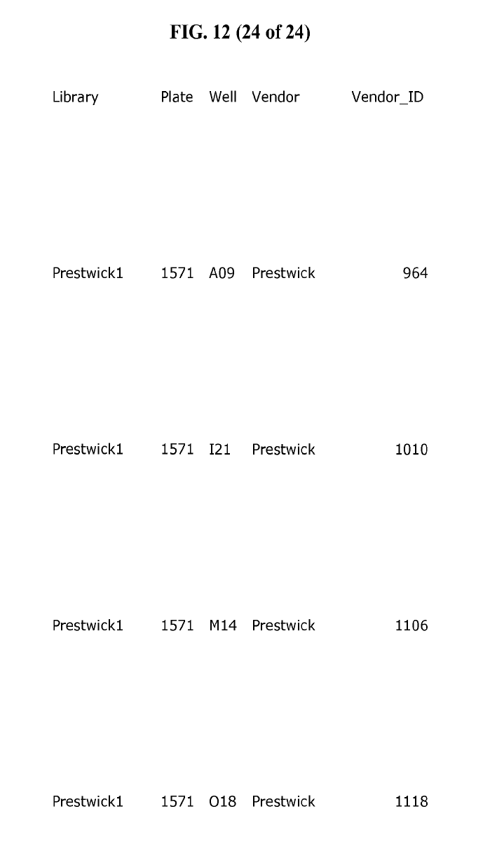

Thiourea appears to be an analog of ISOXYL, an anti-mycobacterial agent reported to be activated by the enzyme EthA, Baeyer-Villier monooxygenase (Dover et al. (2007) Antimicrobial Agents and Chemotherapy 51:1055-1063). PD18 showed an MIC against the E. coli wild type of 25 μg/ml. Prodrug hits molecular structures are presented in FIG. 11, and molecules displaying direct inhibitory activity are listed in FIG. 12.

B. Screen #2

Yet another screen was performed on 147,500 compounds, using the protocol described below. In this screen, hits with antimicrobial activity against the wild type were first identified. These hits were then tested for activity against the BacPool mix of knockout strains described in Example 1. 41 compounds demonstrated direct inhibitory activity, and 6 were identified as candidate prodrug compounds.

Protocol

An E. coli KanR culture was grown in LB broth containing 50 μg/ml Kanamycin for 20 hrs at 37° C. in a shaking incubator. The fresh overnight culture was then diluted 1:10 in fresh media and cultured for 1.5 hrs at 37° C. in a shaking incubator. After growth, the cultures were diluted 1:1000 in fresh media and dispensed into wells of a 384 well microtiter plate (30 μl/well) containing a test compound (final concentration around 50 μg/ml). The plates were then incubated for 20 hrs at 37° C., and then the plates were read for Optical Density at 600 nm and were scored for growth/no growth. Testing was performed in duplicate.

In each plate, column 23 was used a negative control (cells alone, which displayed full growth of E. coli), and column 24 was used as a positive control (cells+Zidovudine (AZT) at 5 μg/ml, which showed complete inhibition of E. coli growth). Before beginning the screen, a Z′-test was run. This was conducted by comparing growth in negative control wells to those with the prodrug AZT at 5 μg/ml. E. coli K12 cells were cultured in LB medium, and exponentially-growing cells were dispensed at 10⁵/ml in 384-well microtiter plates, 30 μl/well. Four plates were used in this experiment. After an overnight incubation at 37° C., the OD₆₀₀ of the plates were read, and the values of each well were used to calculate Z′-factor:

Z′=1−(3SD++3SD−)/(Ave+−Ave−) where:

SD+=positive control standard deviation, SD-=negative control standard deviation, Ave+=positive control average, and Ave-=negative control average. Z′-score obtained was 0.97, suggesting a strong reproducibility of controls throughout the screening.

Hits were identified by calculating the % of inhibition of growth of E. coli measured by OD₆₀₀. The data were analyzed as follows: negative controls (N), positive controls (P), and compounds (X) ODs were separately averaged and the resulting means were used to calculate % of inhibition of E. coli growth:

% of inhibition(% I)=(N−x)/(N−P)*100

Hits were strong if % I>97%; medium if 95<% I<97; and poor if 90<% I<95. Based on this classification, a total of 56 hits (0.04%) of total screened compounds were retrieved, of which 7 were weak, 2 were medium, and 47 were strong. The 56 hits were subsequently tested against the BacPool to check for the presence of potential prodrugs.

Results

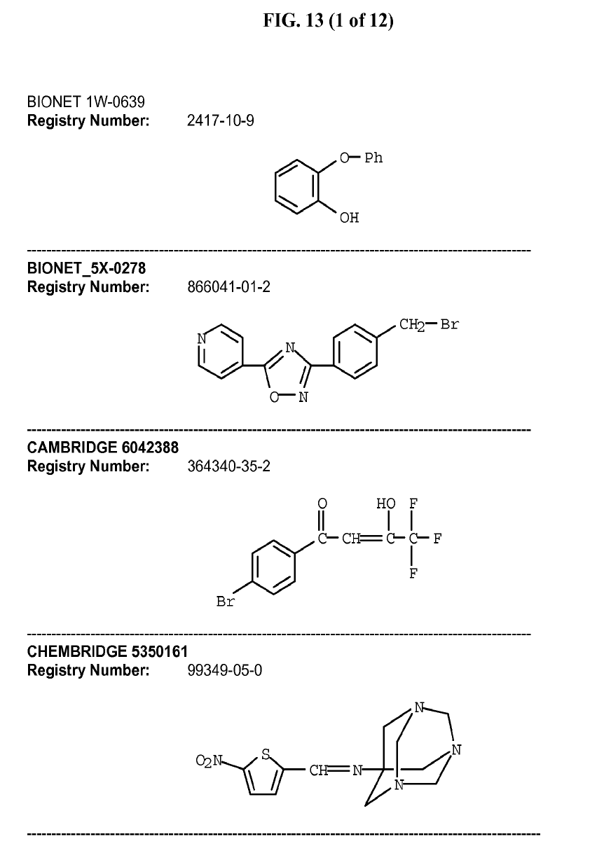

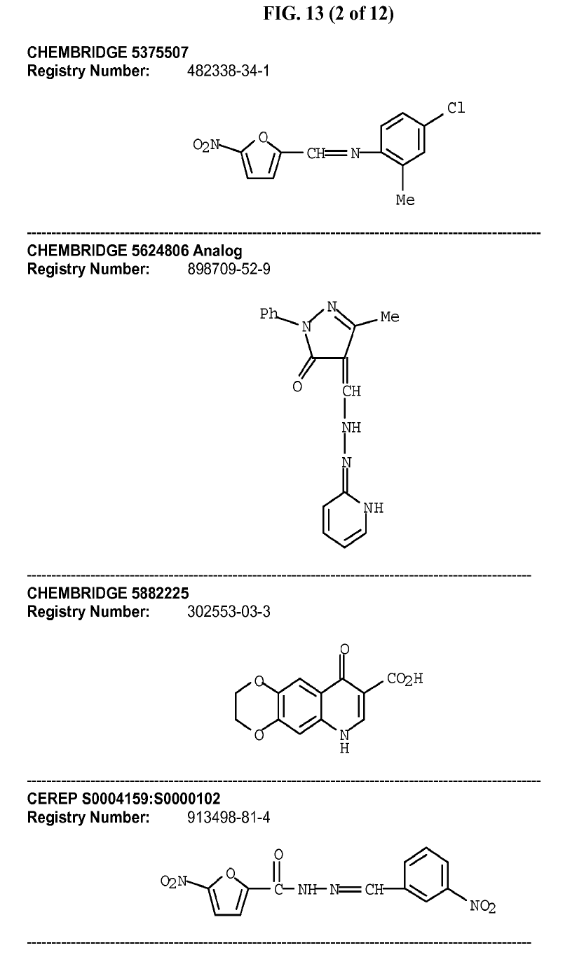

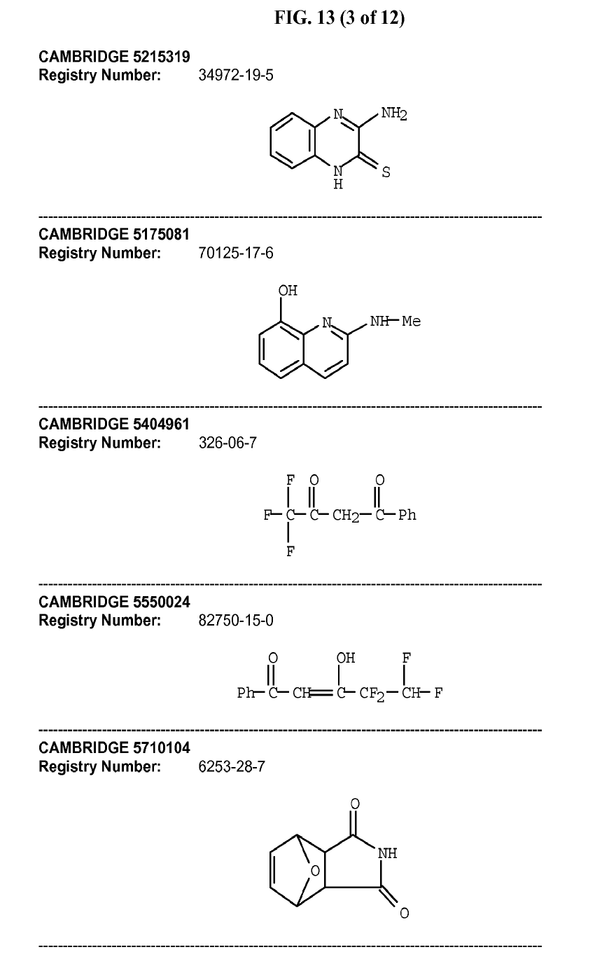

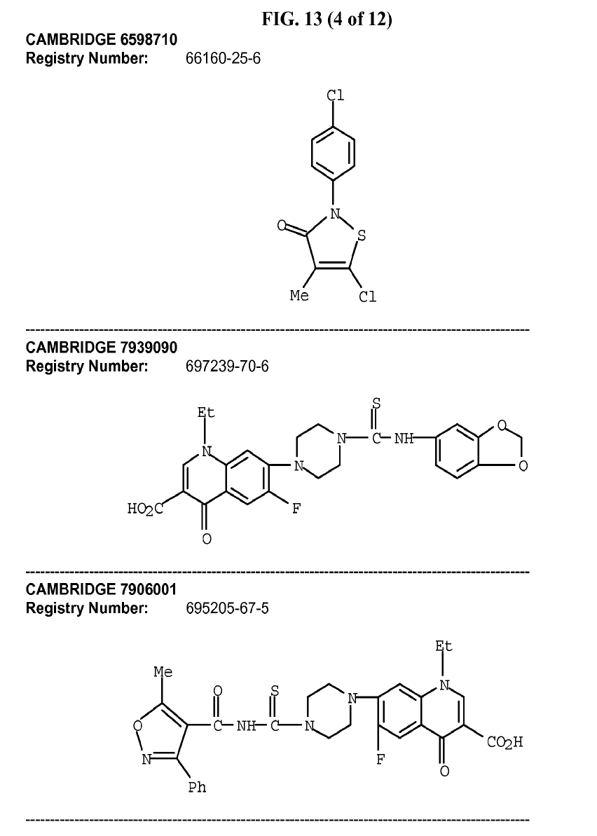

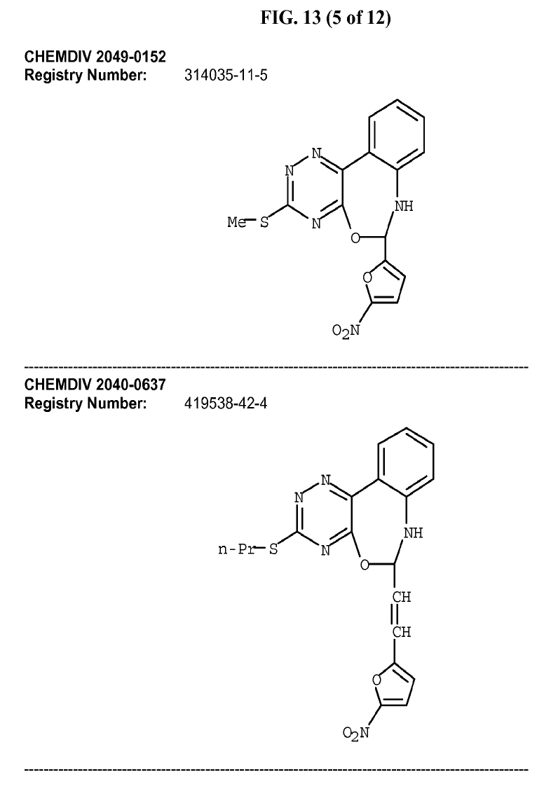

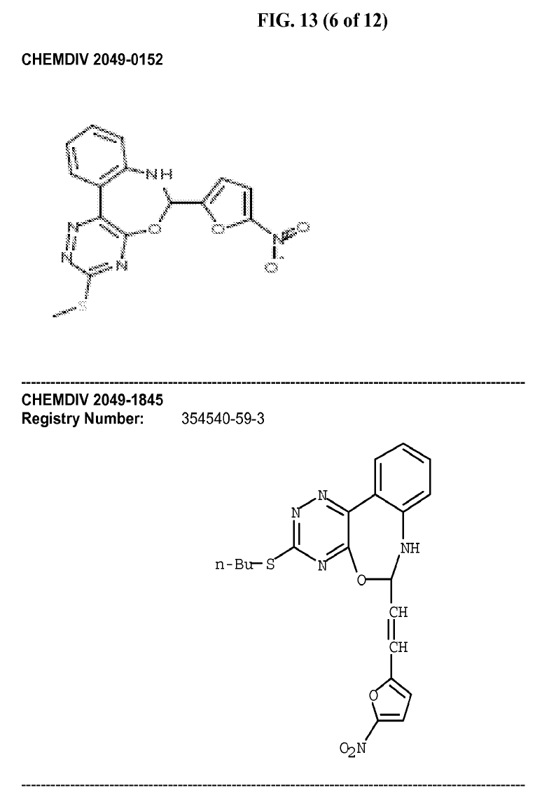

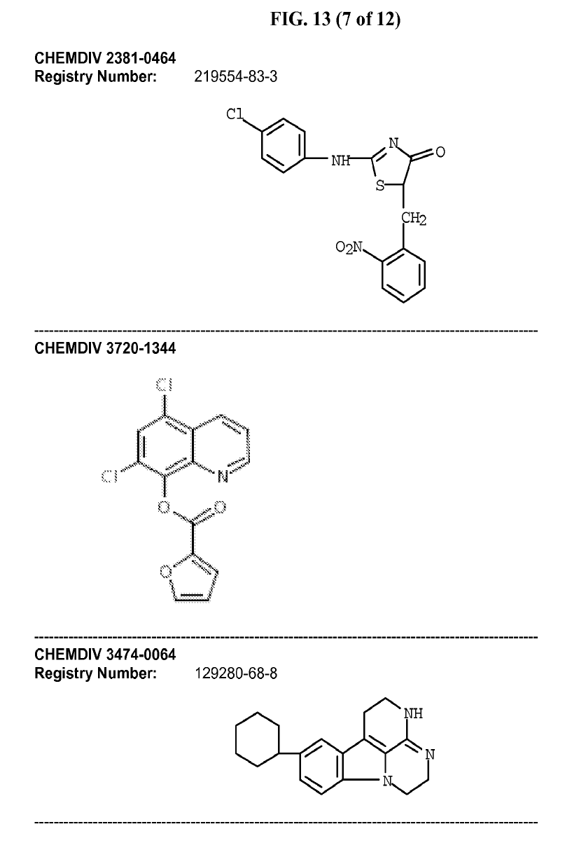

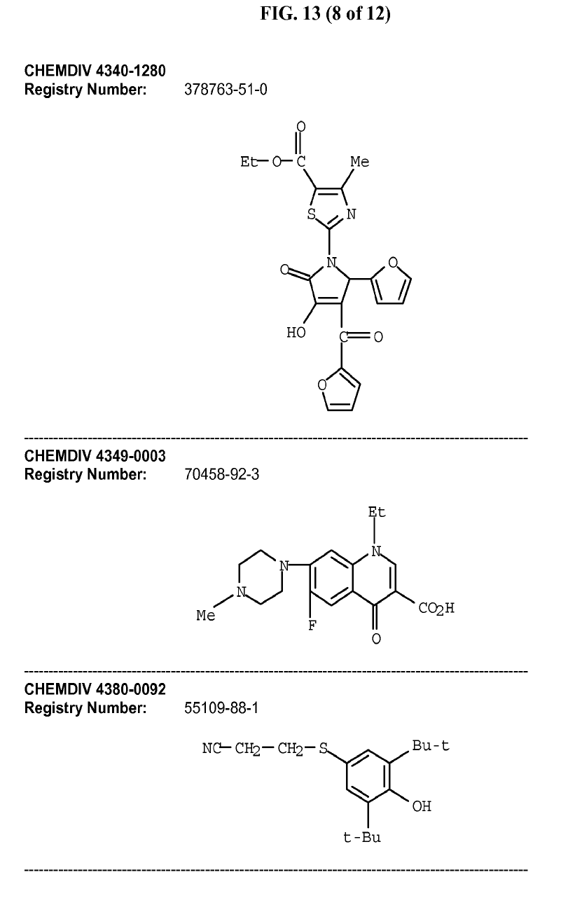

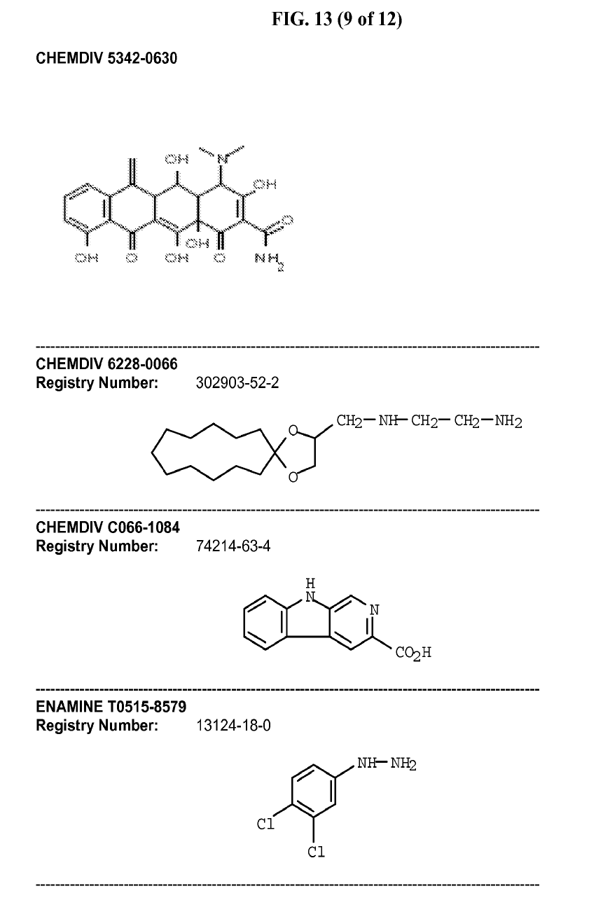

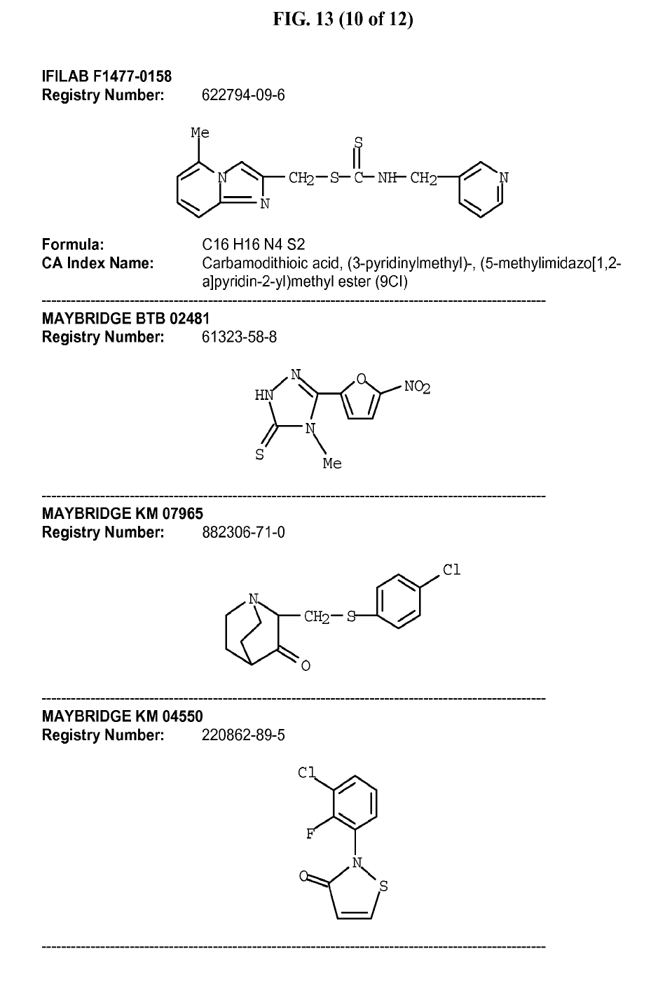

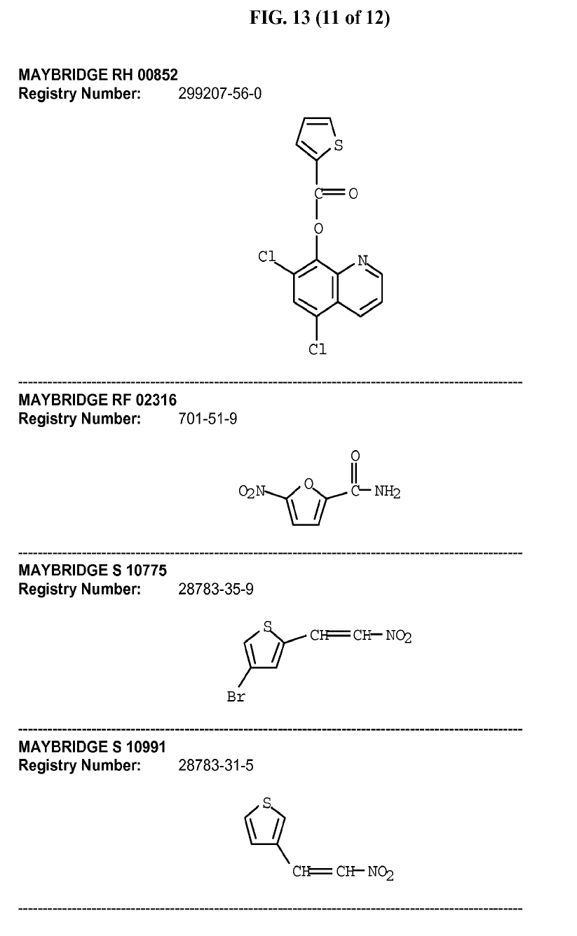

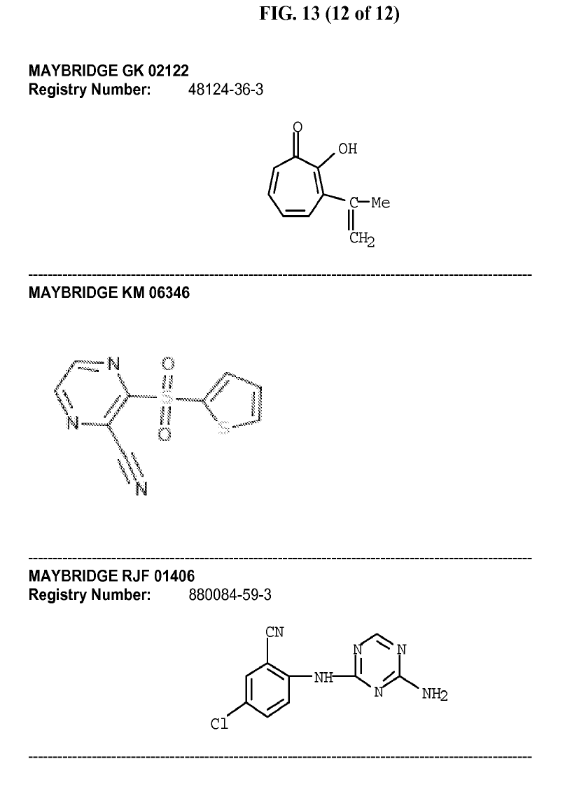

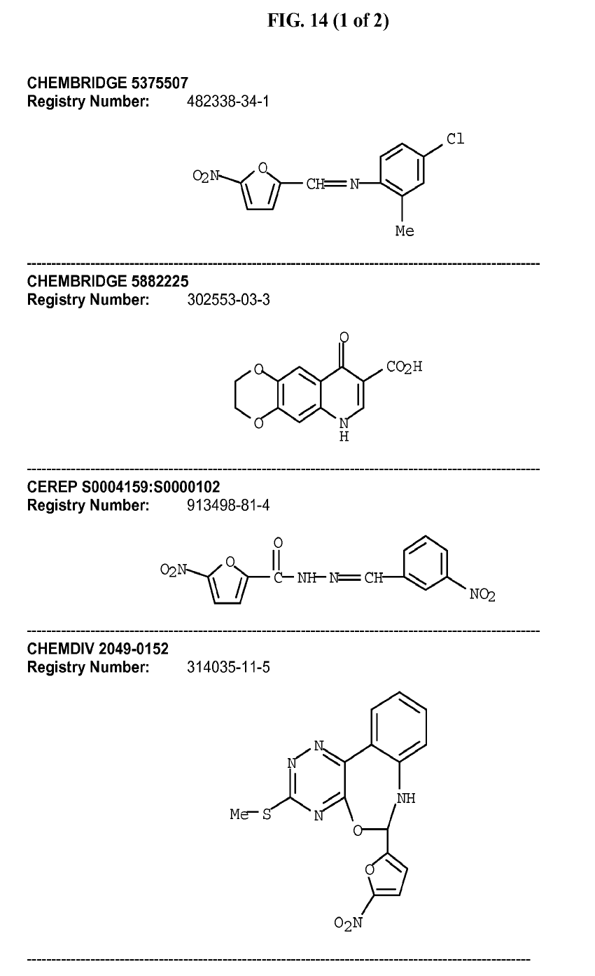

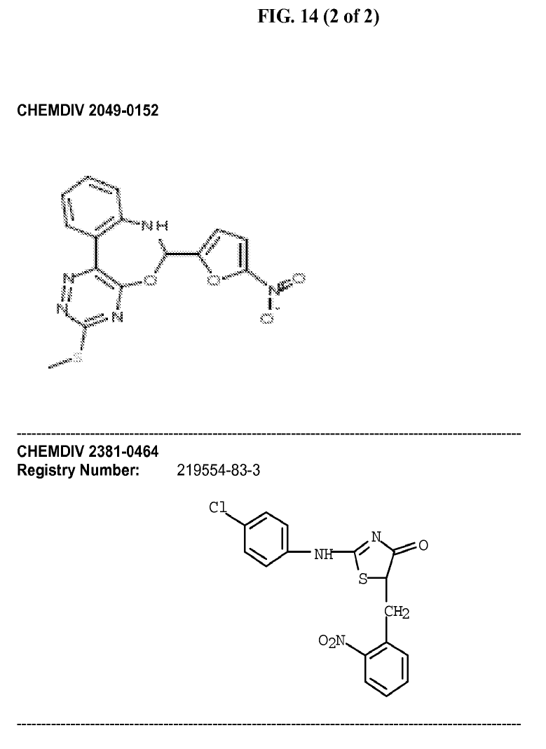

Six hit compounds scored as prodrug hits, and 41 displayed direct activity. Molecular structures of compounds that displayed direct inhibitory activity against the wild type are presented in FIG. 13. Candidate prodrugs are presented in FIG. 14.

Among the prodrug hits several compounds were noted that are known prodrugs or are related chemically to known prodrugs. These included Acetamide, N-(5-nitro-2-thiazolyl)-(8CI,9CI), also known as Nitazole:

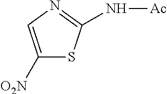

Nitazole is a metrondazole-like compound used as a therapeutic against gram-positive facultative and obligate anaerobic microorganisms as well as gram-negatives except for Psudomonas aeruginosa and Proteus (Kalinichenko et al. (1998) Mikrobiol. Z. 60:83-91). Metronidazole is converted into an active form in bacterial cells by nitroreductases under anaerobic conditions and acts specifically against anaerobic species. Nitazole is also activated by nitroreductases.

To validate the screening, the activity of nitazole against strains with nitrate reductases deleted and in strains overexpressing nitrate reductases were tested. A prodrug activated by a nitrate reductase has lower activity against a strain lacking the enzyme, and a higher activity in an overexpression strain, as compared to a wild type. E. coli has two known nitrate reductases, NfnB and NfsA. Strains tested were wild type (WT) E. coli, two knockout strains lacking the 2 nitroreductases (NfnB−; NfsA−), and against two strains overexpressing the same enzymes (NfnB+ and NfsA+). MIC values for nitazole are shown below.

| Strain | MIC (μg/ml) |
|---|---|
| Wild Type | 12.5 |
| NfnB− | 25 |
| NfsA− | 25 |
| NfnB+ | 1.6 |
| NfsA+ | 6.3 |

These results suggest that the nitrate reductases activate nitazole, confirming its prodrug nature.

Another known prodrug hit was benzoxazole, 2,3-dihydro-5-nitro-2-(5-nitro-2-thienyl)-(9CI):

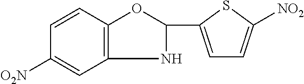

Because this molecule has two nitro groups, whether it could be activated by nitroreductases was assessed using the method described above for nitazole. MIC values for benzoxazole were:

| Strain | MIC (μg/ml) |
|---|---|
| Wild Type | 12.5 |
| NfnB− | 25 |
| NfsA− | 25 |
| NfnB+ | 3.1 |
| NfsA+ | 6.3 |

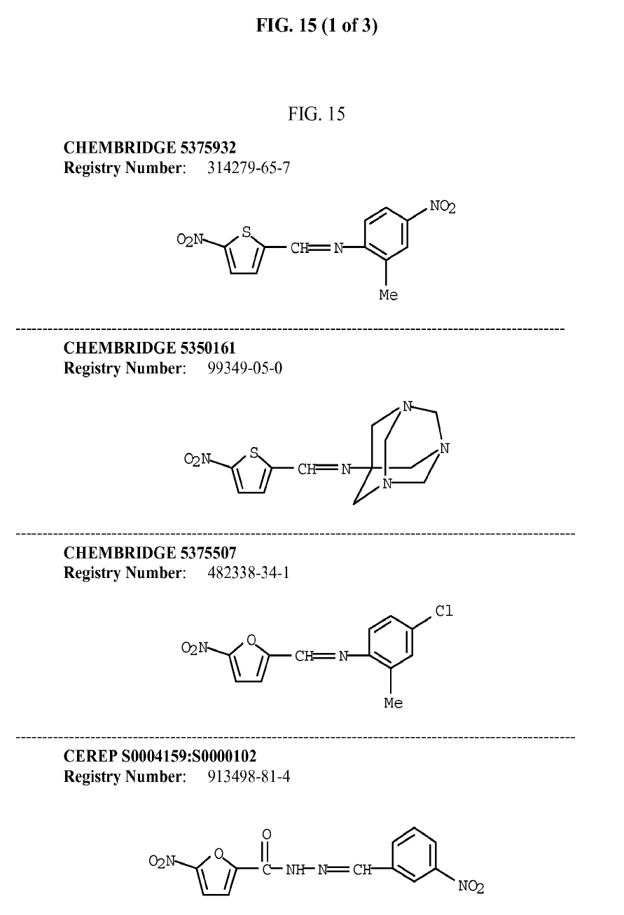

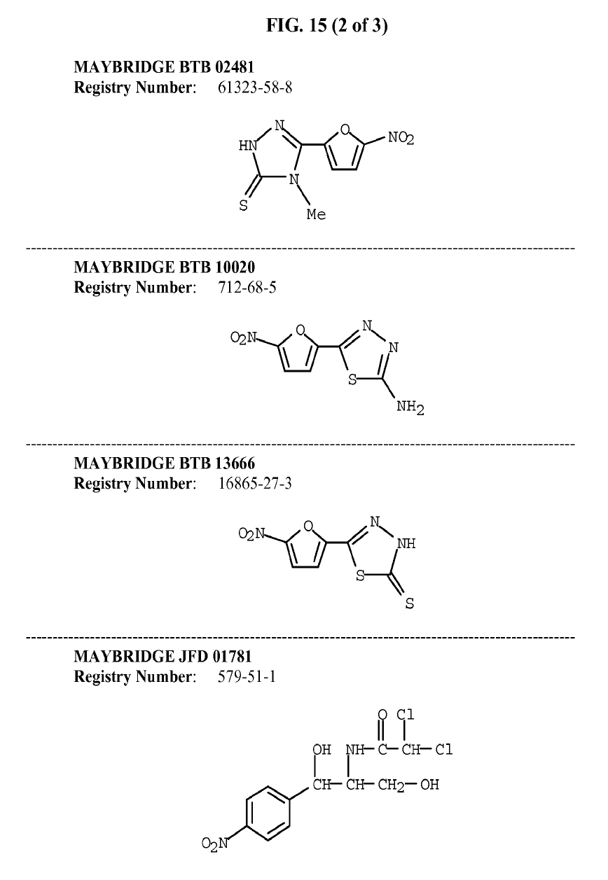

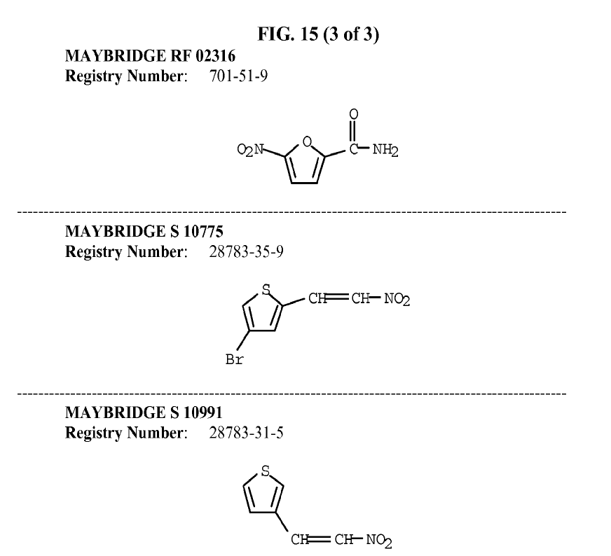

These results indicated that the nitrate reductases activated this compound, as well as suggesting its prodrug nature. Additional compound hits are shown in FIG. 15.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of inhibiting the growth of, or killing, a pathogen, comprising contacting the pathogen with one or more compounds of Formulae I and II, 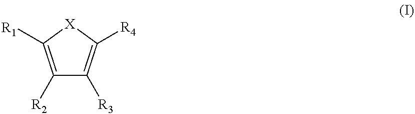
and pharmaceutically acceptable salts, hydrates, and solvates thereof,
wherein:
R₁ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein one or more hydrogens on R₁ can be substituted with 0-5 Rₐ groups;
R₂ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein one or more hydrogens on R₂ can be substituted with 0-5 Rₐ groups;
R₃ is null, —H, halogen, amino, hydroxyl, cyano, nitro, C₁₋₆ alkyl, C₂₋₆-alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, —CH═CHNO₂, —CH₂SC(NH)NH₂, wherein one or more hydrogens on R₃ can be substituted with 0-5 Rₐ groups;
R₄ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl, 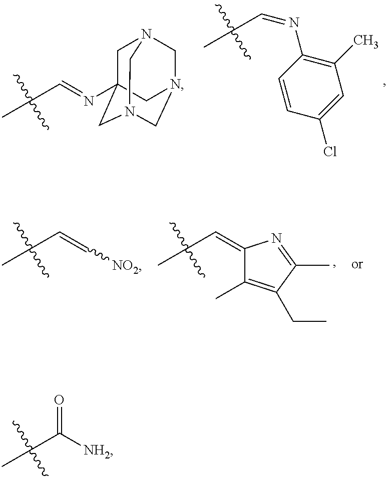
wherein one or more hydrogens on R₄ can be substituted with 0-5 Rₐ groups;
Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl; and
X is O, S, or NH; 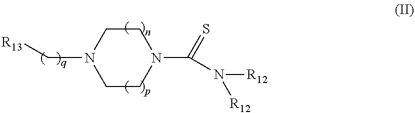
and pharmaceutically acceptable salts, hydrates, and solvates thereof,
wherein:
each R₁₂, independently, is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl, 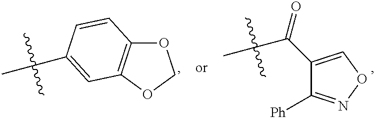
wherein one or more hydrogens on R₁₂ can be substituted with 0-5 R₃ groups;
R₁₃ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl, 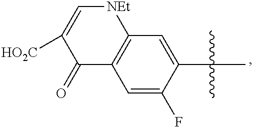
or
wherein one or more hydrogens on R₁₃ can be substituted with 0-5 Rₐ groups;
Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓ(C₁₋₃ alkyl, C₃₋₆ cycloalkyl, or SOₓC₃₋₆ cycloalkyl-C₁₋₃ alkyl;
n is 0 or 1;
p is 1 or 2; and
q is 0 or 1;
thereby inhibiting the growth of, or killing, the pathogen.

2. The method of claim 1, wherein the compound is of Formula I.

3. The method of claim 1, wherein the compound is of Formula II.

4. The method of claim 1, wherein the pathogen is selected from the group consisting of a bacterium, a fungus, a protozoan, a helminth, and a combination thereof.

5. A method of inhibiting the growth of, or killing, a pathogen, comprising contacting the pathogen with one or more analogs of Compounds 1-11: 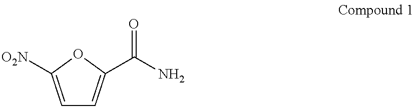
or a pharmaceutically acceptable salt, hydrate, or solvate thereof,
wherein any one or more of —H and —NO₂, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 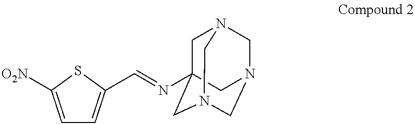
or a pharmaceutically acceptable salt, hydrate, or solvate thereof,
wherein any one or more of —H and —NO₂, attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration; 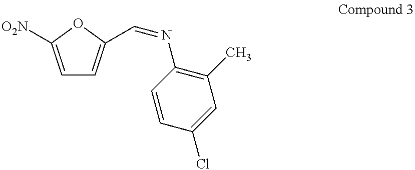
or a pharmaceutically acceptable salt, hydrate, or solvate thereof,
wherein any one or more of —H, —Cl, and —CH₃ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration; 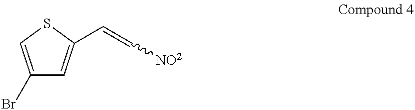
or a pharmaceutically acceptable salt, hydrate, or solvate thereof,
wherein any one or more of —H and —Br can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration; 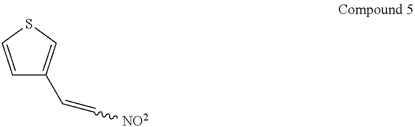
or a pharmaceutically acceptable salt, hydrate, or solvate thereof,
wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; 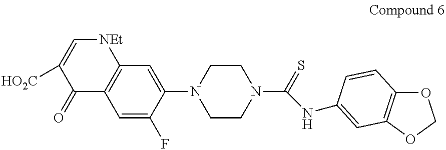
or a pharmaceutically acceptable salt, hydrate, or solvate thereof,
wherein:
any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₄ cycloalkyl-C₁₋₃ alkyl; alkylaryl; aryl; arylalkyl; and heteroaryl;
any one or more of —H and —CH₂CH₃, attached to nitrogen or oxygen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
—H attached to oxygen can be substituted with any one of the following substituents: C₁₋₆ alkyl; C₂₋₆ alkenyl; C₂₋₆ alkynyl; C₃₋₆ cycloalkyl; C₃₋₄ cycloalkyl-C₁₋₃ alkyl; alkylaryl; aryl; arylalkyl; and heteroaryl; 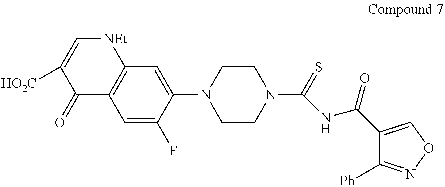
or a pharmaceutically acceptable salt, hydrate, or solvate thereof,
wherein:
any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
any one or more of —H and —CH₂CH₃, attached to nitrogen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
—H attached to oxygen can be substituted with any one of the following substituents: C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl;
the phenyl attached to the isoxazole can be replaced with C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl; 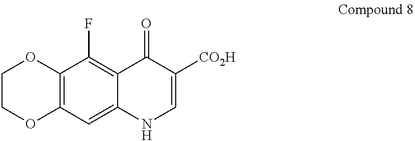
or a pharmaceutically acceptable salt, hydrate, or solvate thereof,
wherein any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
wherein any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; 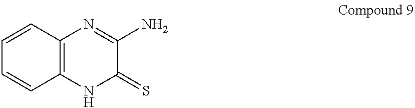
or a pharmaceutically acceptable salt, hydrate, or solvate thereof,
wherein any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
wherein any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃alkyl; alkylaryl; aryl; arylalkyl; and heteroaryl; the C(S) can be substituted with C(O); 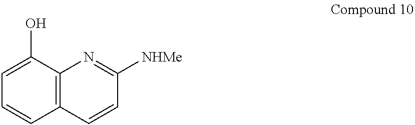
or a pharmaceutically acceptable salt, hydrate, or solvate thereof,
wherein any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
wherein any one or more of —H and —CH₃ attached to nitrogen or oxygen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; 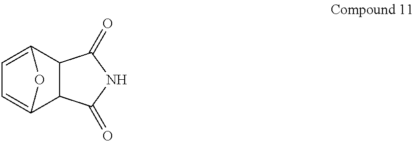
or a pharmaceutically acceptable salt, hydrate, or solvate thereof,
wherein any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; and
wherein any one or more of —H and —CH₃ attached to nitrogen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl,
thereby inhibiting the growth of, or killing, the pathogen.

6. The method of claim 5, wherein the pathogen is selected from the group consisting of a bacterium, a fungus, a protozoan, a helminth, and a combination thereof.

7. A method of inhibiting the growth of, or killing, a pathogen, comprising contacting the pathogen with one or more compounds of one or more of Compounds 1-11: 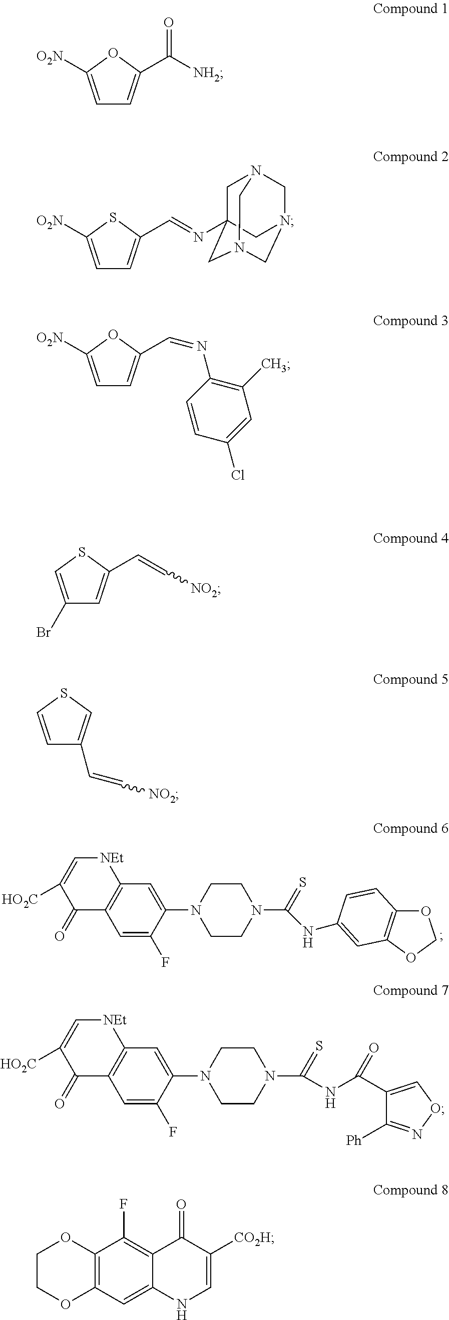 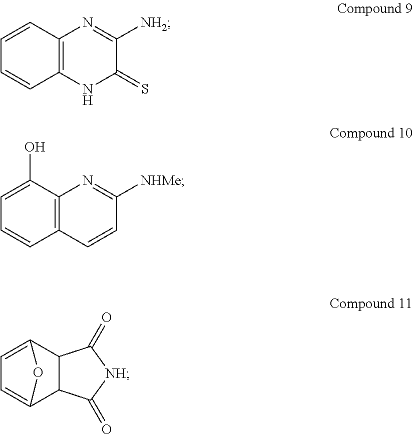
or a pharmaceutically acceptable salt, hydrate, or solvate of Compounds 1-11,
thereby inhibiting the growth of, or killing, the pathogen.

8. The method of claim 7, wherein the pathogen is selected from the group consisting of a bacterium, a fungus, a protozoan, a helminth, and a combination thereof.

9. A method of treating an infection by a pathogen in a subject in need thereof, the method comprising administering to the subject an effective amount of one or more compounds of Formulae I and II, 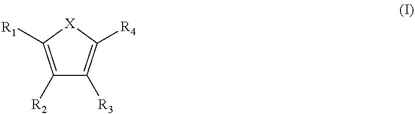
and pharmaceutically acceptable salts, hydrates, and solvates thereof,
wherein:
R₁ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, and heteroaryl, or heteroarylalkyl, wherein one or more hydrogens on R₁ can be substituted with 0-5 Rₐ groups;
R₂ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein one or more hydrogens on R₂ can be substituted with 0-5 Rₐ groups;
R₃ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, —CH═CHNO₂, —CH₂SC(NH)NH₂, wherein one or more hydrogens on R₃ can be substituted with 0-5 Rₐ groups;
R₄ is null, —H, halogen, amino, hydroxyl, cyano, nitro, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl, 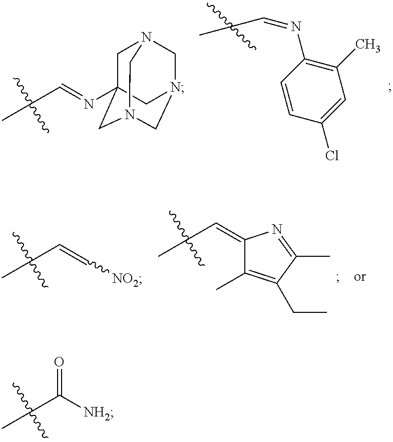
wherein one or more hydrogens on R₄ can be substituted with 0-5 Rₐ groups;
Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl; and
X is O, S, or NH; 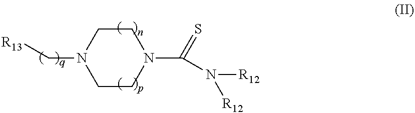
and pharmaceutically acceptable salts, hydrates, and solvates thereof,
wherein:
each R₁₂, independently, is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl, 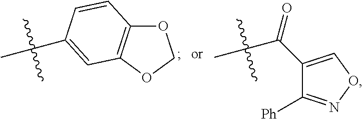
wherein one or more hydrogens on R₁₂ can be substituted with 0-5 Rₐ groups;
R₁₃ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl, 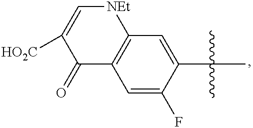
or
wherein one or more hydrogens on R₁₃ can be substituted with 0-5 Rₐ groups;
Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
n is 0 or 1;
p is 1 or 2;
q is 0 or 1;
thereby treating the infection.

10. The method of claim 9, wherein the pathogen is selected from the group consisting of a bacterium, a fungus, a protozoan, a helminth, and a combination thereof.

11. The method of claim 9, wherein the infection is selected from the group consisting of an upper respiratory tract disease, an infection of a catheter, an infection of an orthopedic prostheses, a urinary tract infection, a gastrointestinal infection, a heart valve infection, endocarditis, a skin infection, a chronic wound, and cystic fibrosis.

12. The method of claim 9, wherein the compound is of Formula I.

13. The method of claim 9, wherein the compound is of Formula II.

14. A method of treating an infection by a pathogen in a subject in need thereof, the method comprising administering to the subject an effective amount of one or more analogs of Compounds 1-11: 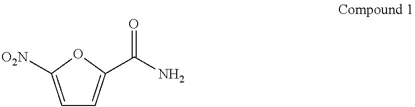
or a pharmaceutically acceptable salt, hydrate, or solvate thereof,
wherein any one or more of —H and —NO₂, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆ alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 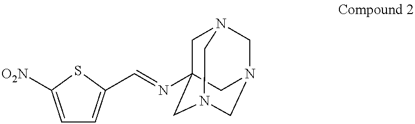
or a pharmaceutically acceptable salt, hydrate, or solvate thereof;
wherein any one or more of —H and —NO₂ attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆ alkyl; —C(O)OC₁₋₆alkyl; C₃₋₆ cycloalkyl; C₃₋₆ cycloalkyl-C₁₋₃alkyl; alkylaryl; aryl; arylalkyl; and heteroaryl; the double bond can be in the E or Z configuration; 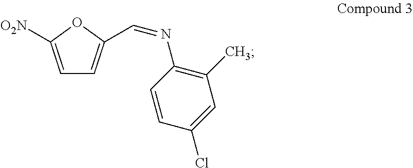
or a pharmaceutically acceptable salt, hydrate, or solvate thereof,
wherein:
any one or more of —H, —Cl, and —CH₃ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration; 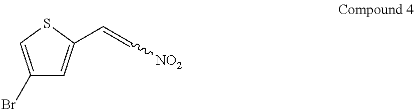
or a pharmaceutically acceptable salt, hydrate, or solvate thereof,
wherein any one or more of —H and —Br can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the double bond can be in the E or Z configuration; 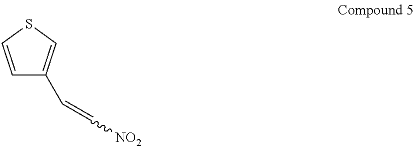
or a pharmaceutically acceptable salt, hydrate, or solvate thereof,
wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; 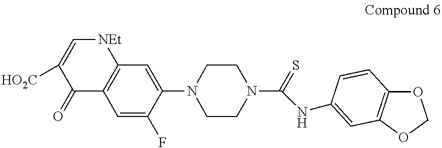
or a pharmaceutically acceptable salt, hydrate, or solvate thereof,
wherein:
any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
any one or more of —H and —CH₂CH₃, attached to nitrogen or oxygen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃alkyl; alkylaryl; aryl; arylalkyl; and heteroaryl; and
—H attached to oxygen can be substituted with any one of the following substituents: C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl; 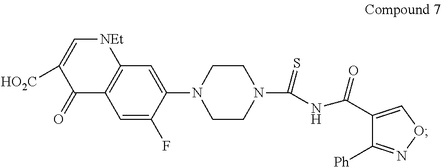
or a pharmaceutically acceptable salt, hydrate, or solvate thereof,
wherein:
any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆ alkyl; —C(O)OC₁₋₆alkyl; C₃₋₆ cycloalkyl; C₃₋₆ cycloalkyl-C₁₋₃ alkylaryl; aryl; arylalkyl; and heteroaryl;
any one or more of —H and —CH₂CH₃, attached to nitrogen, can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
—H attached to oxygen can be substituted with any one of the following substituents: C₁₋₆ alkyl; C₂₋₆ alkenyl; alkynyl; C₃₋₆ cycloalkyl; C₃₋₆ cycloalkyl-C₁₋₃ alkyl; alkylaryl; aryl; arylalkyl; and heteroaryl; and
the phenyl attached to the isoxazole can be replaced with C₁₋₆ alkyl; C₂₋₆ alkenyl; C₂₋₆ alkynyl; C₃₋₆ cycloalkyl; C₃₋₆ cycloalkyl-C₁₋₃ alkyl; alkylaryl; aryl; arylalkyl; and heteroaryl; 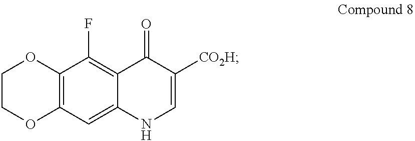
or a pharmaceutically acceptable salt, hydrate, or solvate thereof,
wherein any one or more of —H and —F, attached to carbon, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
wherein any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; 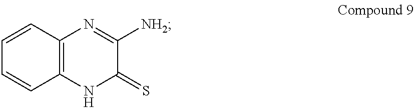
or a pharmaceutically acceptable salt, hydrate, or solvate thereof,
wherein any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
wherein any one or more of —H attached to a nitrogen can be substituted with any one of the following substituents: —NH₂; hydroxyl; C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; the C(S) can be substituted with C(O); 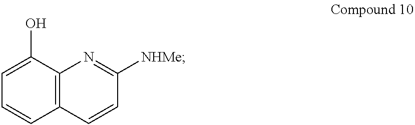
or a pharmaceutically acceptable salt, hydrate, or solvate thereof,
wherein any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
wherein any one or more of —H and —CH₃ attached to nitrogen or oxygen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl; 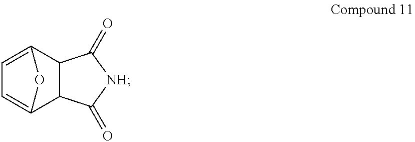
or a pharmaceutically acceptable salt, hydrate, or solvate thereof,
wherein any one or more of —H attached to carbon can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
wherein any one or more of —H and —CH₃ attached to nitrogen can be substituted with any one of the following substituents: C₁₋₆ alkyl; C₂₋₆ alkenyl; C₂₋₆ alkynyl; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; and heteroaryl;
thereby treating the infection.

15. The method of claim 14, wherein the pathogen is selected from the group consisting of a bacterium, a fungus, a protozoan, a helminth, and a combination thereof.

16. The method of claim 14, wherein the infection is selected from the group consisting of an upper respiratory tract disease, an infection of a catheter, an infection of an orthopedic prostheses, a urinary tract infection, a gastrointestinal infection, a heart valve infection, endocarditis, a skin infection, a chronic wound, and cystic fibrosis.

17. A method of treating an infection by a pathogen in a subject in need thereof, the method comprising administering to the subject an effective amount of one or more compounds of one or more of Compounds 1-19: 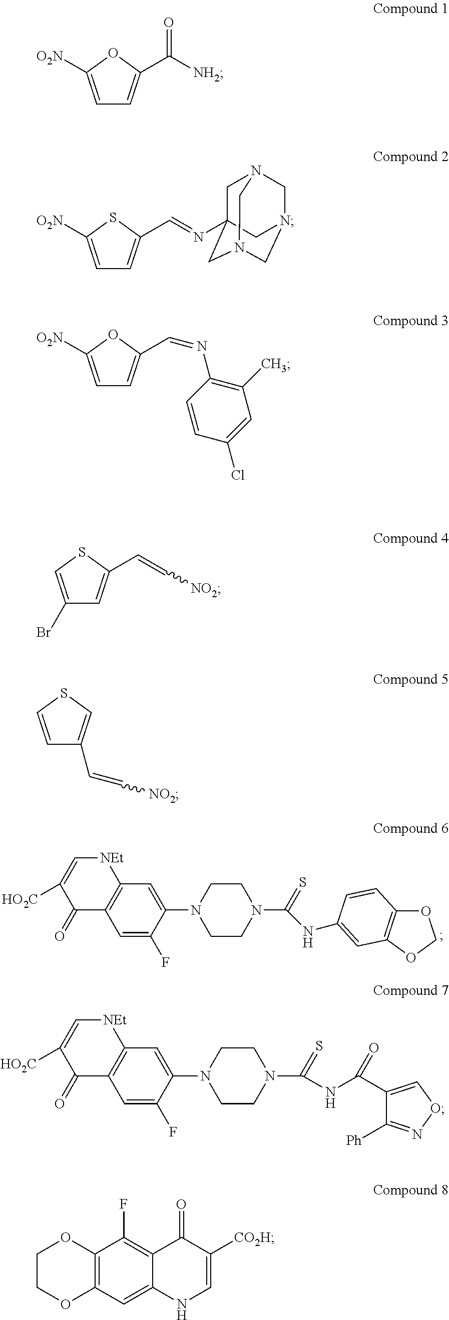 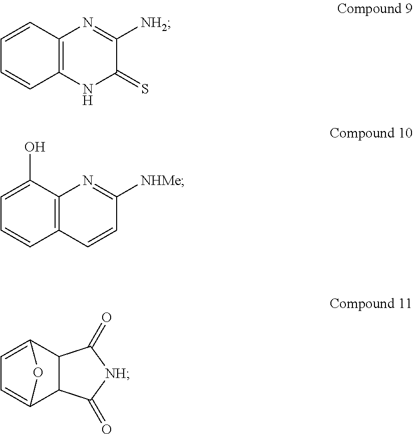
or a pharmaceutically acceptable salt, hydrate, or solvate of Compounds 1-11,
thereby treating the infection.

18. The method of claim 17, wherein the pathogen is selected from the group consisting of a bacterium, a fungus, a protozoan, a helminth, and a combination thereof.

19. The method of claim 17, wherein the infection is selected from the group consisting of an upper respiratory tract disease, an infection of a catheter, an infection of an orthopedic prostheses, a urinary tract infection, a gastrointestinal infection, a heart valve infection, endocarditis, a skin infection, a chronic wound, and cystic fibrosis.
